SEQUENCE LISTING (1 sequence)

SEQ ID NO: 1 - protein, 424 aa, synthetic construct
  REGION 1..424: note = Synthetic
MDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSHPIPDSSPLLQFGGQVRQRYLYTDDAQQTEAHLEIREDGTVGGAADQSPESLLQLKALKPGVIQILGVKTSRFLCQRPDGALYGSLHFDPEACSFRERLLEDGYNVYQSEAHGLPLHLPGNKSPHRDPAPRGPARFLPLPGLPPAPPEPPGILAPQPPDVGSSDPLSMVGGSQGRSPSYES

(19) United States
(12) Patent Application Publication
(10) Pub. No.: US 2025/0302919 A1
(43) Pub. Date: Oct. 2, 2025

(54) PHARMACEUTICAL COMPOSITIONS OF EFRUXIFERMIN

(71) Applicant: AKERO THERAPEUTICS, INC., South San Francisco, CA (US)

(72) Inventors: Timothy P. Rolph, Brookline, MA (US); Mariana N. Dimitrova, Frederick, MD (US); Simon Eisele, Martinsried (DE); James Bostick, Martinsried (DE)

(21) Appl. No.: 18/700,597

(22) PCT Filed: Oct. 12, 2022
(86) PCT No.: PCT/US22/77968

§ 371 Date: Apr. 11, 2024

Related U.S. Application Data
(60) Provisional application No. 63/255,286, filed on Oct. 13, 2021.

(51) Int. Cl. A61K 38/18; A61K 9/19; A61K 47/02; A61K 47/12; A61K 47/18; A61K 47/22; A61K 47/26; A61K 47/38

(52) U.S. Cl. CPC A61K 38/1825; A61K 9/19; A61K 47/02; A61K 47/12; A61K 47/18; A61K 47/183; A61K 47/22; A61K 47/26; A61K 47/38

(57) ABSTRACT

The disclosure provides pharmaceutical compositions comprising Efruxifermin, processes for preparing lyophilized compositions, and methods of use for treating nonalcoholic steatohepatitis (NASH), nonalcoholic fatty liver disease (NAFL), alcoholic steatohepatitis (ASH), alcoholic liver disease (ALD) or alcoholic fatty liver disease (AFL), type 2 diabetes, obesity, hypertriglyceridemia, dyslipidemia, protein misfolding disease, alcohol-related and other cravings or addictions, reversing liver cirrhosis or reducing fibrosis associated with NASH, ASH, ALD AFL, or protein misfolding disease, normalizing liver fat content, reducing elevated blood glucose, increasing insulin sensitivity, and/or reducing uric acid levels.

### CROSS REFERENCE TO RELATED APPLICATIONS AND INCORPORATION BY REFERENCE OF MATERIAL SUBMITTED ELECTRONICALLY

This application claims priority to U.S. Provisional Patent Application No. 63/255,286, filed Oct. 13, 2021, the disclosure of which is hereby incorporated by reference in its entirety.

Incorporated by reference in its entirety is a computer-readable nucleotide/amino acid sequence listing submitted concurrently herewith and identified as follows: 50011_Seqlisting.XML; Size: 2,497 bytes; Created: Oct. 9, 2022.

### FIELD OF THE INVENTION

The disclosure is related to a pharmaceutical composition comprising Efruxifermin (EFX), processes for preparing a lyophilized composition, and methods of use.

### BACKGROUND

Fibroblast growth factor 21 (FGF21) is an endocrine hormone that acts on the liver, pancreas, muscle, and adipose tissue to regulate the metabolism of lipids, carbohydrates, and proteins. Acting as a paracrine hormone, human FGF21 also plays a critical role in protecting cells against stress. These attributes make FGF21 agonism a compelling therapeutic mechanism, but native FGF21 is limited by its short half-life in the bloodstream. The Fc-FGF21 fusion protein, Efruxifermin (EFX), has been genetically engineered to increase human FGF21's half-life (Hecht et al, PLoS One 2012; 7(11): e49345; Stanislaus et al., Endocrinology. 2017; 158(5):1314-1327). However, formulations of EFX are susceptible to post-translational modifications, including formation of charge and size variants, resulting in stability constraints. There is a need in the art for pharmaceutical formulations that provide enhanced stabilization and reduced post-translational modifications of Fc-FGF21 fusion proteins, such as Efruxifermin (EFX).

### SUMMARY

The disclosure provides a pharmaceutical composition comprising Efruxifermin (EFX), a sugar, about 20 to about 200 mM arginine/arginine-HCl or arginine/glutamic acid, and a surfactant. In various aspects, the composition has a pH from about 6.9 to about 8.1. In various aspects, the sugar of the composition is sucrose, glucose, fructose, or maltose. Optionally, the surfactant of the composition is polysorbate-20 or polysorbate-80. In various aspects, the pharmaceutical composition comprises about 25-150 mg/mL EFX; about 120 mM sucrose; about 120 mM Arginine/Arginine-HCl; about 0.06% weight/volume (w/v) polysorbate-20; and about 20 mM Tris-HCl. Optionally, the composition pH is about 7.3.

The composition of the disclosure is, in various instances, lyophilized, although this is not required. In this respect, the disclosure provides a method for reconstituting a lyophilized composition disclosed herein within five minutes, and administering the reconstituted composition to a subject. In various embodiments, the reconstituted composition is maintained at room temperature for up to 10 minutes. The disclosure also provides a dual chamber device comprising any of the compositions disclosed herein and a diluent. In certain aspects, the diluent is water for injection or a buffering agent (e.g., compounded buffer based on the formulations disclosed herein).

The disclosure also provides a pharmaceutical composition comprising EFX, 2.9% L-Lysine, 0.008% weight/volume (w/v) polysorbate-20, and 10 mM Tris. In various aspects, the composition has a pH of 7.8±0.3.

The disclosure also provides a process for preparing lyophilized compositions. In various aspects, the process comprises the following steps: (a) freezing a composition disclosed herein; (b) annealing the composition of step (a) at a temperature of about −5° C. to about −15° C.; (c) primary drying the product of step (b) and d) secondary drying the product of step (c).

The disclosure further provides (a) a method of treating nonalcoholic steatohepatitis (NASH) or nonalcoholic fatty liver disease (NAFL), alcoholic steatohepatitis (ASH), alcoholic liver disease (ALD) or alcoholic fatty liver disease (AFL), type 2 diabetes, obesity, dyslipidemia, alcohol-related and other cravings or addictions, or protein misfolding diseases in a subject in need thereof; (b) a method of normalizing liver fat content in a subject; (c) a method of reversing liver cirrhosis or reducing fibrosis associated with NASH, ASH, ALD, AFL, or protein misfolding disease; (d) a method of reducing blood glucose and/or increasing insulin sensitivity in a subject; and (e) a method of reducing uric acid levels in a subject. The method comprises administering a pharmaceutical composition disclosed herein to a subject in need thereof.

The foregoing summary is not intended to define every aspect of the invention, and additional aspects are described in other sections, such as the Detailed Description. The entire document is intended to be related as an unified disclosure, and it should be understood that all combinations of features described herein are contemplated, even if the combination of features are not found together in the same sentence, or paragraph, or section of this document. In addition, the invention includes, as an additional aspect, all aspects of the invention narrower in scope in any way than the variations specifically mentioned above.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. The terms “comprising,” “having,” “including,” and “containing” are to be construed as open-ended terms unless otherwise noted. If aspects of the invention are described as “comprising” a feature, aspects also are contemplated “consisting of” or “consisting essentially of” the feature. The use of any and all examples, or exemplary language (e.g., “such as”) provided herein, is intended merely to better illustrate the disclosure and does not pose a limitation on the scope of the disclosure unless otherwise claimed. No language in the specification should be construed as indicating any non-claimed element as essential to the practice of the disclosure. Other than in the operating examples, or where otherwise indicated, all numbers expressing quantities should be understood as modified in all instances by the term “about” as that term would be interpreted by the person skilled in the relevant art. With respect to aspects of the invention described or claimed with “a” or “an,” it should be understood that these terms mean “one or more” unless context unambiguously requires a more restricted meaning. With respect to elements described as one or more within a set, it should be understood that all combinations within the set are contemplated.

It should also be understood that when describing a range of values, the disclosure contemplates individual values found within the range. For example, “a pH from about pH 6 to about pH 8,” could be, but is not limited to, pH 6.1, 6.6, 7.2, 7.5, etc., and any value in between such values. In any of the ranges described herein, the endpoints of the range are included in the range. However, the description also contemplates the same ranges in which the lower and/or the higher endpoint is excluded. When the term “about” is used, it means the recited number plus or minus 5%, 10%, or more of that recited number. The actual variation intended is determinable from the context.

Additional features and variations of the invention will be apparent to those skilled in the art from the entirety of this application, including the figures and detailed description, and all such features are intended as aspects of the invention. Likewise, features of the invention described herein can be re-combined into additional aspects that also are intended as aspects of the invention, irrespective of whether the combination of features is specified as an aspect of the invention. The entire document is intended to be related as a unified disclosure, and it should be understood that all combinations of features described herein (even if described in separate sections) are contemplated, even if the combination of features is not found together in the same sentence, or paragraph, or section of this document. Also, only such limitations which are described herein as critical to the invention should be viewed as such; variations of the invention lacking limitations which have not been described herein as critical are intended as aspects of the invention. The use of section headings is merely for the convenience of reading; it should be understood that all combinations of features described herein are contemplated.

### BRIEF DESCRIPTION OF THE DRAWINGS

FIG. 1 shows EFX schematic structure with disulfide bonds and polypeptide chains.

FIGS. 2A-2B show visually observed gel formation and Schlieren phase separation of EFX in various formulations at pH≤6.5.

FIG. 3 shows the initial dynamic viscosity of EFX 100 mg/mL in formulations F1 to F20 (except F11) at 400 s⁻¹ shear rate and time zero.

FIGS. 4A-4B show the dynamic viscosity of EFX in formulations F1 to F20 (except F11) after 3 days at 40° C./75% relative humidity (RH) followed by storage for 21 months at 2-8° C. at 10 s⁻¹ shear rate (FIG. 4B is a zoomed in view from 0 to 50 cP).

FIGS. 5A-5C. FIG. 5A shows EFX in formulations susceptible to forming gels and phase separation, demonstrated non-Newtonian shear thinning effect. FIGS. 5B and 5C show that, in contrast, formulations characterized with low viscosities at pH 6.5, demonstrated Newtonian behavior.

FIG. 6 shows an Example AEX HPLC Chromatogram of EFX in Tris/Lys formulation F18.

FIG. 7 shows distribution of charge variants in EFX formulation F18 separated by imaged capillary isoelectric focusing (icIEF).

FIGS. 8A-8C. FIG. 8A shows the formation of EFX charge variants, measured by AEX-HPLC as a function of time at 25° C. in various formulations. EFX in F33 demonstrates lowest rates of purity loss (as % main peak area) over time. FIG. 8B shows the relative abundance in percent total area of basic charge variants (pre-peaks) by AEX-HPLC at time zero, 1 week, and 1 month at 25° C./60% RH for formulations F1 to F20. FIG. 8C shows the relative abundance in percent total area of acidic charge variants (post-peaks) by AEX-HPLC at time zero, 1 week, and 1 month at 25° C./60% RH for formulations F1 to F20.

FIG. 9 shows the formation of EFX charge variants, measured by AEX-HPLC as a function of time. The assay was performed at a target temperature of 5° C., providing results which are representative of a temperature range of 2-8° C. EFX in F33 demonstrates the slowest rate of purity loss (as % decrease of main peak area) over time.

FIG. 10 shows a Size Exclusion HPLC Profile of EFX in Formulation F18.

FIG. 11 shows the formation of size variants (HMWS, LMWS) of EFX when stored at 25° C., quantitated by SE-HPLC of various formulations. EFX in F33 demonstrated the lowest rate of purity loss per week of the formulations examined.

FIG. 12 shows the formation of size variants (HMWS, LMWS) of EFX when stored at 2-8° C., quantitated by SE-HPLC of various formulations. EFX in F33 demonstrated the lowest rate of purity loss in the formulations examined.

FIG. 13 is an example electropherogram of non-reduced CE-SDS of EFX (F18).

FIG. 14 shows size variants (HMWS, LMWS) of EFX by CE-SDS (non-reduced) in various formulations during storage at 25° C. EFX in F33 demonstrated the lowest rate of purity loss in the formulations tested.

FIG. 15 shows size variants of EFX by CE-SDS (non-reduced) in various formulations during storage at 2-8° C.

FIG. 16 shows analysis of EFX by Reversed-Phase HPLC.

FIG. 17 shows formation of size variants (HMWS, LMWS) in various formulations of EFX stored at 25° C., as measured by RP-HPLC. EFX in F18 and F33 demonstrated the slowest rates of purity loss.

FIG. 18 shows formation of size variants (HMWS, LMWS) in various formulations of EFX at 2-8° C., as measured by RP-HPLC. EFX in F18 and F33 demonstrated the slowest rates of purity loss.

FIG. 19 shows SEC-MALLS chromatogram of EFX in F18 showing main peak and peaks corresponding to dimer and HMW Species.

FIG. 20 shows a sedimentation coefficient distribution profile of EFX in F18 and F33 analyzed by SV-AUC.

FIG. 21 shows a concentration response curve for EFX in F18 as measured by iLite FGF21 cell-based potency bioassay. Data displayed is mean RLU (relative luminescence units) of EFX dilutions plated in triplicate on a single plate. The error bars denote standard deviation of triplicate RLU values.

FIG. 22 shows the potency of EFX by cell-based bioassay in various formulations stored at 25° C. as a function of time, and compared to EFX standard. EFX in F33 demonstrated the lowest rates of potency loss.

FIG. 23 shows the potency of EFX by cell-based bioassay in various formulations stored at 2-8° C. as a function of time, and compared to EFX standard. EFX in F33 demonstrated the lowest rates of potency loss of the formulations tested.

FIG. 24 shows the second derivative FTIR spectrum of EFX in F18 and F33 formulations.

FIG. 25 shows far-UV CD spectrum of EFX in formulations F18 and F33.

FIG. 26 shows near UV CD spectrum of EFX in F18 and F33.

FIGS. 27A-27B show μDSC thermogram of EFX in F18 (FIG. 27A) and F33 (FIG. 27B) after baseline correction.

FIG. 28 shows μDSC thermograms of EFX F18 and F33 heated twice to 50° C.

FIG. 29 shows an example of a lyophilization process design without annealing (vial).

FIGS. 30A-30B show an example of a lyophilization process design with an annealing process step conducted for 5 hours at −10° C. (FIG. 30A: vial) and an annealing process step for 10 hours at −15° C. (FIG. 30B: dual chamber device).

FIG. 31 shows reconstitution times of lyophilized EFX in selected formulations.

FIG. 32 shows reconstitution times of lyophilized F33 in vials, after incorporation of an annealing step into the freeze-drying process (10 hours at −5° C.).

FIGS. 33A-33B show reconstitution times of lyophilized F33 in dual chamber devices, after incorporation of an annealing step into the freeze-drying process (10 hours at−15° C.).

FIG. 34 shows specific surface area of lyophilized cake produced from formulations comprising the same components as F33 but with different concentrations of EFX (F1: 50 mg/ml EFX; F2: 28 mg/ml EFX), by freeze drying with and without an annealing step, as measured by BET (Brunauer, Emmett and Teller method). Lyophilized cakes were produced using either an annealing step of −5° C. for 10 hours or −10° C. for 5 hours and compared to cakes produced without an annealing step during freeze drying (NA process design).

FIG. 35 shows sections of freeze-dried cake for SEM-EDX analysis.

FIG. 36 shows the distribution and median cross-sectional area of lyophilized cake pores by SEM-EDX presented as Box-and-Whisker plots.

FIG. 37 shows the structure of lyophilized cake by SEM is improved by incorporation of an annealing step in the freeze-drying cycle. FR1 and FR2 represent formulation F33 at 50 mg/mL and 28 mg/mL protein concentration, respectively.

FIG. 38 shows a table of long-term stability of lyophilized EFX at 25° C. in Formulations F15, F16, F17, and F33.

FIG. 39 is a line graph illustrating the persistence of EFX administered in various formulations to rats as described in Example 7. Concentration (ng/mL) is indicated on the y-axis, while time (hours) is noted on the x-axis.

FIG. 40 is a chart summarizing pharmacokinetic parameters indicative of overall systemic exposure (AUC) and highest concentration in systemic circulation (Cmax) of EFX administered in various formulations described in the Examples.

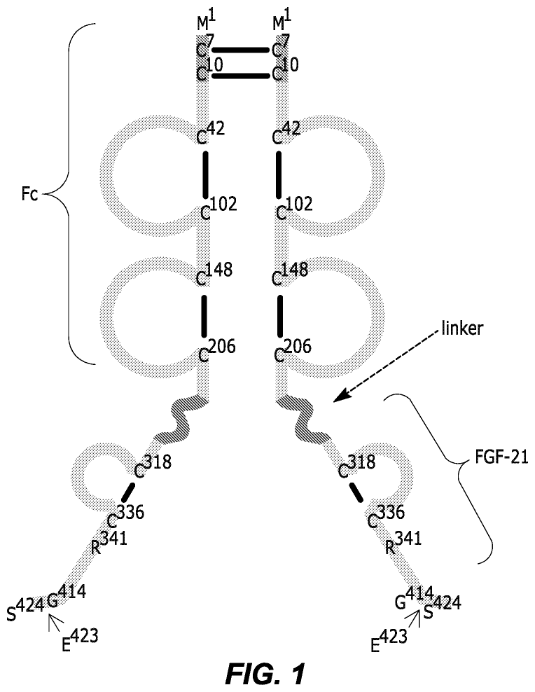

### DETAILED DESCRIPTION

EFX is an FGF21 variant fused to an Fc domain. Surprisingly, EFX displays unique properties that complicate formulation and storage of the protein. Parental injectable biologics are frequently formulated at slightly acidic to neutral pH (e.g., pH 5.2 to pH 6.9) to minimize posttranslational modifications, such as deamidation. Unexpectedly, EFX adopts dramatically different viscoelastic properties at pH below 6.5, manifesting gel-like behavior, phase separation, and loss of fluidity. These features challenge subcutaneous administration of the product and development of injectable biologics. In addition, at or below pH 6.9, EFX compositions demonstrated a propensity for protein aggregation and clipping/fragmentation, along with formation of visible and subvisible particles. These changes are also undesirable for injectable biologics, since they may be associated with safety (particularly immunogenicity) and stability concerns. The materials and methods described herein provide a significant technical advantage by providing formulations of EFX that are suitable for injection and stable when stored, e.g., as a liquid under refrigerated conditions (2-8° C.) and as a lyophile under refrigerated and ambient conditions (25° C.). In various aspects of the disclosure, the formulation described herein provides enhanced EFX conformational stability (by, e.g., preventing or minimizing phase separation, rigid gel formation, non-Newtonian viscoelastic behavior, and aggregation and/or particle formation), reduces post-translational modifications (e.g., charge and/or size variants), and imparts beneficial solution properties to EFX compositions.

### Definitions

The terminology used herein is for the purpose of describing particular embodiments only and is not intended to be limiting.
“AEX HPLC” refers to anion-exchange high-performance liquid chromatography.
“ASH” refers to alcoholic steatohepatitis.
“ALD” refers to alcoholic liver disease.
“AFL” refers to alcoholic fatty liver disease.
“BET” refers to Brunauer, Emmett and Teller method.
“CE-SDS” refers to capillary electrophoresis with sodium dodecyl sulfate.
“EFX” refers to Efruxifermin.
“HMWS” refers to High Molecular Weight Species.
“icIEF” refers to imaged capillary isoelectric focusing.
“LMWS” refers to Low Molecular Weight Species.
“NAFL” refers to nonalcoholic fatty liver disease.
“NASH” refers to nonalcoholic steatohepatitis.
“RH” refers to relative humidity.
“RP-HPLC” refers to reversed-phase high-performance liquid chromatography.
“SE-HPLC” refers to size exclusion high-performance liquid chromatography.
“SEM-EDX” refers to Scanning Electron Microscopy with Energy Dispersive X-Ray Spectroscopy.
“SV-AUC” refers to Sedimentation Velocity Analytical Ultracentrifugation.
“pI” refers to isoelectric point.

The disclosure provides a pharmaceutical composition comprising Efruxifermin. In various aspects, the composition comprises EFX, a sugar, arginine/arginine-HCl or arginine/glutamic acid (e.g., at a concentration of about 20-200 mM), and a surfactant. The composition has a pH from about 6.9 to about 8.1. In alternative aspects, the composition comprises EFX, L-Lysine, a surfactant (e.g., polysorbate-20), and Tris, at a pH of about 7.8±0.3. Also provided is a process for preparing a lyophilized composition comprising EFX. The disclosure further provides methods of using the pharmaceutical compositions described herein for treating a variety of disorders, such as (but not limited to) nonalcoholic steatohepatitis (NASH), nonalcoholic fatty liver disease (NAFL), alcoholic steatohepatitis (ASH), alcoholic liver disease (ALD) or alcoholic fatty liver disease (AFL), type 2 diabetes, obesity, hypertriglyceridemia, dyslipidemia, protein misfolding diseases, craving and addiction, as well as reducing fibrosis associated with NASH, reversing liver cirrhosis or reducing fibrosis associated with NASH, ASH, ALD, AFL or protein misfolding disease, normalizing liver fat content, reducing blood glucose, increasing insulin sensitivity, and/or reducing uric acid levels. Various aspects of the composition and methods are described in more detail below. The use of subheadings is merely for the convenience of the reader, and should not be construed as limiting the disclosure in any way. The entire document is intended to be read as a unified disclosure, and all combinations of features described below are contemplated.

EFX is a 92.1 kDa, long-acting fibroblast growth factor 21 (FGF21) analogue generated by the fusion of human immunoglobulin IgG1 Fc fragment via a poly glycine-serine linker to a variant of human FGF21. Each molecule contains one dimeric Fc domain and two modified FGF21 polypeptide chains. EFX has 8 disulfide bonds, 6 intra-chain and 2 inter-chain as depicted in Error! Reference source not found. Two of the intrachain disulfide bonds are in the FGF21 polypeptide between Cys318 and Cys336, one for each monomer. Three modifications were introduced into the FGF21 sequence at L341R, P414G, and A423E (corresponding to L98R, P171G, and A180E relative to mature, human FGF21). These modifications 1) decrease susceptibility to in vivo proteolytic degradation, 2) increase affinity for β-Klotho, and 3) decrease the propensity to aggregate (Hecht et al., PLoS One 2012; 7(11): e49345; Stanislaus et al., Endocrinology. 2017; 158(5):1314-1327).

EFX comprises the amino acid sequence set forth in SEQ ID NO: 1. EFX has been further described in U.S. Pat. Nos. 8,034,770; 8,410,051; 8,642,546; 8,361,963; 9,273,106; 10,011,642; 8,188,040; 8,835,385; 8,795,985; 8,618,053; and 11,072,640; or International Patent Publication Nos. WO2009149171 and WO2010129503, the disclosures of which are incorporated herein by reference in their entireties.

EFX may be present in the pharmaceutical composition in any suitable amount. In various aspects, the concentration of EFX in the pharmaceutical composition is about 25 mg/ml to about 150 mg/ml. For example, the concentration of EFX in the pharmaceutical composition is at least about 25 mg/ml, at least about 30 mg/ml, at least about 35 mg/ml, at least about 40 mg/ml, at least about 45 mg/ml, at least about 50 mg/ml, or at least about 70 mg/ml, and not greater than about 150 mg/ml, not greater than about 140 mg/ml, not greater than about 130 mg/ml, not greater than about 120 mg/ml, not greater than about 110 mg/ml, or not greater than about 100 mg/ml. In exemplary aspects, the composition comprises EFX at a concentration of about 28 mg/ml. In exemplary aspects, the composition comprises EFX at a concentration of about 50 mg/ml. In exemplary aspects, the composition comprises EFX at a concentration of about 70 mg/ml. In exemplary aspects, the composition comprises EFX at a concentration of about 100 mg/ml.

The pharmaceutical composition comprising EFX may be a liquid, lyophilized, or gel formulation.

The pharmaceutical compositions described herein comprises a sugar. Suitable sugars include, but are not limited to, sucrose, fructose, maltose, glucose, galactose, lactose, sorbitol, mannitol, or a combination thereof. The sugar may be present in the composition at a concentration of about 10 mM to about 250 mM, or about 20 mM to about 220 mM, or about 50 mM to about 220 mM, or about 80 to about 220 mM, or about 120 mM. In some aspects, the concentration of sugar in the pharmaceutical composition is at least about 10 mM, at least about 20 mM, at least about 30 mM, at least about 40 mM, at least about 50 mM, at least about 60 mM, at least about 70 mM, at least about 80 mM, at least about 90 mM, at least about 100 mM, at least about 110 mM, or at least about 120 mM, and not greater than about 250 mM, not greater than about 240 mM, not greater than about 230 mM, not greater than about 220 mM, not greater than about 210 mM, not greater than about 200 mM, not greater than about 190 mM, not greater than about 180 mM, not greater than about 170 mM, not greater than about 160 mM, not greater than about 150 mM, not greater than about 140 mM, or not greater than about 130 mM.

Optionally, the pharmaceutical compositions described herein comprises at a sugar at a concentration of about 50 mM, about 80 mM, about 100 mM, about 110 mM, about 115 mM, about 120 mM, or about 125 mM, about 130 mM, about 135 mM, about 140 mM, about 145 mM, about 150 Mm, about 155 mM, about 160 mM, about 165 mM, about 175 mM, about 180 mM, about 185 mM, about 190 mM, about 210 mM, about 215 mM, about 220 mM, about 225 mM, about 230 mM, or about 235 mM.

In various aspects, the pharmaceutical composition is a liquid or lyophilized form. An exemplary liquid or lyophilized pharmaceutical composition (e.g., a lyophilized form prepared by freeze drying any of the liquid formulations described herein) comprises sucrose at a concentration of about 50 mM to about 220 mM, such as about 80 mM or about 120 mM.

In various aspects, the sugar is trehalose. For example, in some aspects, the pharmaceutical composition is a gel formulation and the sugar is trehalose. An exemplary gel formulation comprises trehalose at a concentration of about 180 mM to about 250 mM, such as 220 mM.

In various aspects, the pharmaceutical formulation comprises an amino acid, such as arginine, arginine/arginine-HCl, arginine/glutamic acid, glycine, glutamine, asparagine, or lysine. In various aspects, the composition comprises arginine/arginine-HCl. In various aspects, the arginine/arginine-HCl is present at a ratio of about 1:10 arginine/arginine-HCl to about 1:100 arginine/arginine-HCl. In some aspects, the arginine/arginine-HCl is at a ratio of about 1:30 arginine/arginine-HCl to about 1:50 arginine/arginine-HCl. In various aspects, the arginine/arginine-HCl is present at a ratio of about 1:10 arginine/arginine-HCl, about 1:20 arginine/arginine-HCl, about 1:30 arginine/arginine-HCl, about 1:40 arginine/arginine-HCl, about 1:50 arginine/arginine-HCl, about 1:60 arginine/arginine-HCl, about 1:70 arginine/arginine-HCl, about 1:80 arginine/arginine-HCl, about 1:90 arginine/arginine-HCl, or about 1:100 arginine/arginine-HCl. In various aspects, the composition comprises about 20 mM to about 200 mM arginine/arginine-HCl. For example, the concentration of arginine/arginine-HCl in the pharmaceutical composition is, in various aspects, at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM, and not greater than about 200 mM, not greater than about 180 mM, not greater than about 175 mM, not greater than about 160 mM, not greater than about 155 mM, not greater than about 150 mM, not greater than about 145 mM, not greater than about 140 mM, not greater than about 135 mM, not greater than about 130 mM, not greater than about 125 mM not greater than about 120 mM, not greater than about 110 mM, not greater than about 100 mM, not greater than about 90 mM, not greater than about 80 mM, not greater than about 70 mM, or not greater than about 60 mM. In a representative aspect of the disclosure, the composition comprises about 120 mM arginine/arginine-HCl. In another representative aspect of the disclosure, the composition comprises about 80 mM arginine/arginine-HCl. In various aspects, the pharmaceutical composition is a gel form. An exemplary gel pharmaceutical composition is free of one or more amino acid(s) (i.e., does not contain an amino acid, such as arginine, arginine/arginine-HCl, arginine/glutamic acid, glycine, glutamine, asparagine, or lysine).

In various aspects, the composition comprises arginine/glutamic acid or arginine/glutamate. As used herein, “glutamic acid” and “glutamate” can be used interchangeably. In various aspects, the arginine/glutamic acid is present at a ratio of about 1:10 arginine/glutamic acid to about 1:100 arginine/glutamic acid. In various aspects, the arginine/glutamic acid is present at a ratio of about 1:10 arginine/glutamic acid, about 1:20 arginine/glutamic acid, about 1:30 arginine/glutamic acid, about 1:40 arginine/glutamic acid, about 1:50 arginine/glutamic acid, about 1:60 arginine/glutamic acid, about 1:70 arginine/glutamic acid, about 1:80 arginine/glutamic acid, about 1:90 arginine/glutamic acid, or about 1:100 arginine/glutamic acid. In some aspects, the arginine/glutamic acid is present at a ratio of about 1:30 arginine/glutamic acid to about 1:50 arginine/glutamic acid.

In various aspects, the composition comprises from about 20 mM to about 200 mM arginine/glutamic acid. For example, the total concentration of arginine/glutamic acid in the pharmaceutical composition is optionally at least about 20 mM, at least about 30 mM, at least about 35 mM, at least about 40 mM, at least about 45 mM, or at least about 50 mM, at least about 55 mM, at least about 60 mM, at least about 65 mM, at least about 70 mM, at least about 75 mM, at least about 80 mM, at least about 85 mM, at least about 90 mM, at least about 95 mM, or at least about 100 mM, and not greater than about 200 mM, not greater than about 180 mM, not greater than about 175 mM, not greater than about 170 mM, not greater than about 165 mM, not greater than about 160 mM, not greater than about 155 mM, not greater than about 150 mM, not greater than about 145 mM, not greater than about 140 mM, not greater than about 135 mM, not greater than about 130 mM, not greater than about 125 mM, not greater than about 120 mM, not greater than about 115 mM, not greater than about 110 mM, not greater than about 105 mM, not greater than about 100 mM, not greater than about 90 mM, not greater than about 80 mM, not greater than about 70 mM, or not greater than about 60 mM. In exemplary aspects, the composition comprises arginine/glutamic acid at a total concentration within the range of 80-150 mM or 90-150 mM, such as about 80 mM or about 120 mM.

In various aspects, the amino acid is lysine (e.g., L-Lysine or L-Lysine-HCl). Any disclosure herein relating to L-Lysine also applies to Lysine-HCl. In various aspects, the composition comprises about 0.1%-10% lysine. For example, the concentration of lysine in the pharmaceutical composition is optionally at least about 0.1%, at least about 0.5%, at least about 1%, at least about 1.5%, or at least about 2%, and not greater than about 10%, not greater than about 9%, not greater than about 8%, not greater than about 7%, not greater than about 6%, not greater than about 4%, or not greater than about 3%. In exemplary aspects, the composition comprises lysine at a concentration of about 2.9%. In various aspects, the composition comprises about 6.8 mM-684.0 mM L-Lysine. For example, the concentration of L-Lysine in the pharmaceutical composition is optionally at least about 6.8 mM, at least about 34.2 mM, at least about 68.4 mM, at least about 102.6 mM, or at least about 136.8 mM, and not greater than about 684.0 mM, not greater than about 615.6 mM, not greater than about 547.2 mM, not greater than about 478.8 mM, not greater than about 410.4 mM, not greater than about 273.6 mM, or not greater than about 205.2 mM. In exemplary aspects, the composition comprises L-Lysine at a concentration of about 198.3 mM. In various aspects, the composition comprises about 5.5 mM-547.5 mM Lysine-HCl. For example, the concentration of Lysine-HCl in the pharmaceutical composition is optionally at least about 5.5 mM, at least about 27.4 mM, at least about 54.8 mM, at least about 82.1 mM, or at least about 109.5 mM, and not greater than about 547.5 mM, not greater than about 492.7 mM, not greater than about 438.0 mM, not greater than about 383.2 mM, not greater than about 328.5 mM, not greater than about 218.0 mM, or not greater than about 164.2 mM. In exemplary aspects, the composition comprises Lysine-HCl at a concentration of about 158.8 mM.

In various aspects, the composition comprises an alkalizing buffering agent, such as Tris (tromethamine) and/or Tris-HCl. As used herein, “Tris” and “tromethamine” can be used interchangeably. In various aspects, the composition comprises about 1-50 mM Tris. For example, the concentration of Tris in the pharmaceutical composition is optionally at least about 1 mM, at least about 5 mM, at least about 10 mM, and not greater than about 15 mM, not greater than about 20 mM, not greater than about 25 mM, not greater than about 30 mM, not greater than about 35 mM, not greater than about 40 mM, not greater than about 45 mM, or not greater than about 50 mM. In exemplary aspects, the composition comprises Tris at a concentration of about 10 mM. In various aspects, the composition comprises about 1-50 mM Tris-HCl. For example, the concentration of Tris-HCl in the pharmaceutical composition is optionally at least about 1 mM, at least about 5 mM, at least about 10 mM, and not greater than about 15 mM, not greater than about 20 mM, not greater than about 25 mM, not greater than about 30 mM, not greater than about 35 mM, not greater than about 40 mM, not greater than about 45 mM, or not greater than about 50 mM. In exemplary aspects, the composition comprises Tris-HCl at a concentration of about 10 mM. In various aspects, the composition comprises both about 1-50 mM Tris and 1-50 mM Tris-HCl.

The pharmaceutical composition described herein comprises, in various aspects, a surfactant. Optionally, the surfactant is a nonionic surfactant. Exemplary surfactants include, but are not limited to polysorbate 20 (PS20), polysorbate 40 (PS40), polysorbate 60 (PS60), polysorbate 80 (PS80), poloxamer 188, poloxamer 407, polyoxyethylene, or a combination thereof. In various aspects, the surfactant is polysorbate 20, polysorbate 40, polysorbate 60, or polysorbate 80. In an exemplary aspect, the surfactant is polysorbate 80. In another exemplary aspect, the surfactant is polysorbate 20.

In various embodiments, the formulation further comprises polyethylene glycol (PEG) of any molecular weight, such as PEG 3350, PEG 4000, PEG 6000, or PEG1000 (e.g., PEG 3350 or PEG 4000). For example, the formulation, in various aspects, comprises about 0.05% to about 5% PEG (e.g., PEG 4000), optionally about 0.15% to about 1.5% PEG (e.g., PEG 4000), such as about 0.1% to about 1% PEG (e.g., PEG 4000) or about 0.5% PEG (e.g., PEG 4000). Alternatively, in various embodiments, the formulation comprises hydroxypropyl methylcellulose (HPMC) or carboxymethyl cellulose (CMC) or a salt thereof, such as sodium hydroxypropyl methylcellulose (Na-HPMC) or sodium carboxymethyl cellulose (Na-CMC). In this respect, the formulation optionally comprises about 0.05% to about 5% CMC or HPMC (or salt thereof), optionally about 0.15% to about 1.5% HPMC (e.g., Na-HPMC) or CMC (e.g., Na-CMC), such as about 0.1% to about 1% HPMC (e.g., Na-HPMC) or CMC (e.g., Na-CMC) or about 0.5% HPMC (e.g., Na-HPMC) or CMC (e.g., Na-CMC). In various aspects, the formulation comprises a mixture of PEG and CMC (or salt thereof) or HPMC (or salt thereof), such as these components in any of the amounts described herein. Optionally, the formulation comprises PEG, HPMC (or salt thereof), and CMC (or salt thereof).

The pharmaceutical composition described herein may comprise one surfactant or multiple surfactants in different ratios. In some aspects, a surfactant is included at a concentration of about 0.001% to about 1% w/v (or about 0.002% to about 0.5%). In some aspects, the pharmaceutical composition comprises a surfactant at a concentration of at least about 0.001%, at least about 0.002%, at least about 0.003%, at least about 0.004%, at least about 0.005%, at least about 0.007%, at least about 0.01%, or at least about 0.05%, and no more than about 0.1%, no more than about 0.2%, no more than about 0.3%, no more than about 0.4%, no more than about 0.5%, no more than about 0.6%, no more than about 0.7%, no more than about 0.8%, no more than about 0.9%, or no more than about 1.0% w/v. In some aspects, the pharmaceutical composition comprises a surfactant at a concentration of about 0.001%, about 0.002%, about 0.003%, about 0.004%, about 0.005%, about 0.006%, about 0.007%, about 0.008%, about 0.009%, about 0.01%, about 0.05%, about 0.1%, about 0.2%, about 0.3%, about 0.4%, about 0.5%, about 0.6%, about 0.7%, about 0.8%, about 0.9%, or about 1% w/v. In an exemplary aspect, the composition comprises a surfactant at a concentration of about 0.004% to about 0.1% w/v. In some aspects, the pharmaceutical composition comprises polysorbate 20 or polysorbate 80, optionally at a concentration of 0.004% to about 0.1% w/v. In some aspects, the surfactant is polysorbate 20, and the polysorbate 20 is present in a concentration of about 0.06% w/v. Alternatively, the polysorbate 20 is present at a concentration of about 0.008% (w/v).

In various aspects, the composition also may comprise a buffering agent. Suitable buffers include, but are not limited to, a Tris-HCl buffer, a sodium glutamate/glutamic acid buffer, a glycylglycine/glycylglycine-HCl buffer, a histidine buffer, or a citrate buffer (or a combination thereof). In various aspects, the composition comprises about 5 mM to about 200 mM buffer. For example, the concentration of Tris-HCl buffer in the pharmaceutical composition is optionally at least about 5 mM, at least about 10 mM, at least about 15 mM, at least about 20 mM, at least about 25 mM, or at least about 30 mM, and not greater than about 200 mM, not greater than about 180 mM, not greater than about 160 mM, not greater than about 140 mM, not greater than about 120 mM, not greater than about 100 mM, not greater than about 80 mM, not greater than about 60 mM, or not greater than about 50 mM. In various aspects, the buffer is a Tris-HCl buffer, which is optionally included at a concentration of about 10 mM to about 50 mM. In a representative aspect of the disclosure, the composition comprises about 20 mM Tris-HCl buffer. For a pharmaceutical composition which is a gel formulation, in various embodiments, the pharmaceutical composition may comprise a sodium phosphate buffer, a sodium succinate/succinic acid buffer, or a sodium acetate/acetic acid buffer.

Optionally, the pH of the pharmaceutical composition is about 6 to about 8.1. In various aspects, the pH of the pharmaceutical composition is from about 6.9 to about 8.1. In various aspects, the pH of the pharmaceutical composition is from about 7 to about 8, such as from about 7.0 to about 7.8, or about 7.2 to about 7.4, or about 7.5 to about 8. In some aspects, the pH of the pharmaceutical composition is about 7.3 (e.g., 7.3±0.3). In some aspects, the pH of the pharmaceutical composition is about 7.8 (e.g., 7.8±0.3).

Stability of a protein composition is characterized by examining one or more properties of the pharmaceutical composition, and can be examined at any desired timepoint following formulation, including time points after the composition is stored under any of a variety of temperatures or conditions. Stable compositions in the context of the disclosure generally exhibit, for example, minimal or reduced phase separation, minimal or reduced formation of gel with rigid consistency, Newtonian viscoelastic behavior, minimal or reduced EFX degradation products, and/or minimal or reduced post-translational modifications to EFX (e.g. minimal or reduced charge and/or size variants). Optionally, the pharmaceutical composition exhibits one or more of these properties when stored as a liquid under refrigeration (2-8° C.) (optionally storage for 21 months) and as a lyophile under more stressful ambient conditions (25° C.).

The pharmaceutical composition described herein minimizes unwanted charged variant species and size variant species of EFX, which provides a significant technical advantage for manufacture, storage, distribution and self-administration of the product by patients at home. Charge variants are forms of EFX with differing charge distribution (i.e., more acidic or basic variants of EFX) which may form as a result of post-translational modifications. In various aspects, the composition comprises no more than about 40% charged variant species when stored between −30° C. to −20° C. for up to 24 months. Charge variants of EFX may be measured using any of a number of techniques, such as by AEX-HPLC and icIEF. Using AEX-HPLC, EFX charge variants are characterized by the percentage abundance of pre-peaks on chromatographs (basic variants, or EFX charge variants with less negative charges on their surface), main-peak, and post-peaks (acidic variants, or EFX charge variants with more negative charges on their surface). AEX-HPLC is further described in Example 3. Alternatively, charge variants of EFX may be resolved using icIEF based on isoelectric point (pI) of EFX or charge variants and measured as the percentage abundance of pre-peaks (acidic variants), main-peak, and post-peaks (basic peaks) on icIEF electropherograms. Materials and methods relating to icIEF are further described in Example 3. In various aspects, the composition is a liquid composition and comprises no more than about 40%, no more than about 35%, no more than about 30%, no more than about 25%, no more than about 20%, no more than about 10%, no more than about 5%, no more than about 1%, no more than about 0.1%, or no more than about 0.01% of charged variants, optionally when stored between −30° C. to −20° C. for up to 24 months (i.e., the liquid composition comprises no more than this level of charged variants when tested between time 0 and 24 months under storage conditions comprising a temperature between −30° C. to −20° C.). In exemplary aspects, the liquid composition comprises no more than about 40% acidic charged variant species when stored between −30° C. to −20° C. for up to 24 months.

In various aspects, the pharmaceutical composition is a liquid or lyophilized composition and preferably comprises no more than about 40%, no more than about 35%, no more than about 30%, no more than about 25%, no more than about 20%, no more than about 10%, no more than about 5%, no more than about 1%, no more than about 0.1%, or no more than about 0.01% of charged variants when stored between 2° C. to 8° C. for up to 9 months (i.e., the liquid or lyophilized composition comprises no more than this level of charged variants when tested between time 0 and 9 months under storage conditions comprising a temperature between 2° C. to 8° C.). In exemplary aspects, the liquid or lyophilized composition comprises no more than about 40% acidic charged variant species when stored at about 2-8° C. for up to 9 months.

In various aspects, the pharmaceutical composition is a liquid or lyophilized composition and comprises no more than about 40%, no more than about 35%, no more than about 30%, no more than about 25%, no more than about 20%, no more than about 10%, no more than about 5%, no more than about 1%, no more than about 0.1%, or no more than about 0.01% charged variants when stored at about 20-30° C. for up to 4 weeks (i.e., the liquid or lyophilized composition comprises no more than this level of charged variants when tested between time 0 and 4 weeks under storage conditions comprising a temperature of about 20-30° C./60% Relative Humidity). In exemplary aspects, the liquid or lyophilized composition comprises no more than about 40% acidic charged variant species when stored at about 25° C. for up to 4 weeks.

In various aspects, the pharmaceutical composition is a lyophilized composition and comprises no more than about 40%, no more than about 35%, no more than about 30%, no more than about 25%, no more than about 20%, no more than about 10%, no more than about 5%, no more than about 1%, no more than about 0.1%, or no more than about 0.01% charged variants when stored at about 20-30° C. for up to 14 months (i.e., the lyophilized composition comprises no more than this level of charged variants when tested between time 0 and 14 months under storage conditions comprising a temperature of about 20-30° C./60% Relative Humidity). In exemplary aspects, the lyophilized composition comprises no more than about 40% acidic charged variant species when stored at about 25° C. for up to 14 months.

Size variants in the context of the disclosure refer to aggregation or formation of High Molecular Weight Species (HMWS) and fragmentation or formation of Low Molecular Weight Species (LMWS) of EFX. Size variants of EFX may be measured using any of a number of techniques, such as by size exclusion high-performance liquid chromatography (SE-HPLC), capillary electrophoresis with sodium dodecyl sulfate (CE-SDS, reduced and non-reduced), or reversed-phase HPLC (RP-HPLC), sedimentation velocity analytical ultracentrifugation (SV-AUC) and sodium dodecyl-sulfate polyacrylamide gel electrophoresis (SDS-PAGE).

Using SE-HPLC, EFX size variants are characterized by detecting EFX homodimer as a main species, the predominant chromatographic peak, and low levels of dimer (comprising two EFX homodimers) and high molecular weight (HMW) EFX size variants on a HPLC profile. Materials and methods relating to SE-HPLC are further described in Example 3.

Using CE-SDS under denaturing conditions, EFX size variants are characterized by the migration of peaks on an electropherogram, as detected by UV absorbance at 220 nm. Using this analysis, non-reduced, denatured EFX shows intact protein as main peak, while single chain and low molecular weight species migrate before the main peak as pre-peaks and aggregates/HMW size variants appear after the main peak as post-peaks. Materials and methods relating to CE-SDS are further described in Example 3.

Using RP-HPLC, EFX size variants are characterized by detecting eluted EFX protein peaks with a UV absorbance detector at 280 nm. Using this analysis, size variants are visible as pre- or post-peaks resolved from the main peak on the chromatogram. Materials and methods relating to RP-HPLC are further described in Example 3.

In various aspects, the pharmaceutical composition is a liquid or lyophilized composition and preferably comprises no more than about 10%, no more than about 9%, no more than about 8%, no more than about 7%, no more than about 6%, no more than about 5%, no more than about 4%, no more than about 3%, no more than about 2%, no more than about 1%, no more than about 0.1%, or no more than about 0.01% of EFX size variants when stored at a temperature of about 20-30° C., such as about 25° C., for up to 20 weeks (i.e., the liquid composition comprises no more than this level of size variant species when tested between time 0 and 20 weeks under storage conditions at this temperature). In exemplary aspects, the liquid composition comprises no more than about 10% EFX size variant species when stored at about 25° C. for up to 20 weeks. In exemplary aspects, the lyophilized composition comprises about 0% EFX size variant species (i.e., no EFX size variant species are detected) when stored at about 25° C. for up to 20 weeks.

In various aspects, the pharmaceutical composition is a liquid or lyophilized composition and preferably comprises no more than about 10%, no more than about 9%, no more than about 8%, no more than about 7%, no more than about 6%, no more than about 5%, no more than about 4%, no more than about 3%, no more than about 2%, no more than about 1%, no more than about 0.1%, or no more than about 0.01% of EFX size variants when stored at a temperature between about 2-8° C. for up to 14 months (i.e., the liquid composition comprises no more than this level of size variant species when tested between time 0 and 14 months under storage conditions comprising a temperature of between about 2-8° C.). In exemplary aspects, the liquid composition comprises no more than about 10% EFX size variant species when stored between about 2-8° C. for up to 14 months. In exemplary aspects, the lyophilized composition comprises about 0% EFX size variant species (i.e., no EFX size variant species are detected) when stored between about 2-8° C. for up to 14 months.

In various aspects, the pharmaceutical composition is a liquid or lyophilized composition and preferably comprises no more than about 20%, no more than about 15%, no more than about 10%, no more than about 5%, no more than about 4%, no more than about 3%, no more than about 2%, no more than about 1%, no more than about 0.1%, or no more than about 0.01% of EFX size variants when stored at a temperature between about 20-30° C. (e.g., about 25° C.) for up to 4 weeks (i.e., the liquid composition comprises no more than this level of size variant species when tested between time 0 and 4 weeks under storage conditions comprising a temperature of about 25° C.). In exemplary aspects, the liquid composition comprises no more than about 20% EFX size variant species when stored at about 25° C. for up to 4 weeks. In exemplary aspects, the lyophilized composition comprises about 0% EFX size variant species (i.e., no EFX size variant species are detected) when stored between about 25° C. for up to 14 months.

In various aspects of the disclosure, the pharmaceutical composition is a lyophilized composition. When lyophilized, the residual moisture content of the lyophilized product is optionally about 1% or less (e.g., about 0.5% or less). In various aspects, the lyophilized formulation is reconstituted with an appropriate diluent to form a reconstituted composition of lyophilized EFX, which is contemplated by the disclosure. In this regard, the disclosure also provides methods of reconstituting the pharmaceutical compositions disclosed herein. The method comprises (a) reconstituting the lyophilized pharmaceutical composition disclosed herein within about five minutes and (b) administering the reconstituted composition to a subject. Optionally, step (b) comprises subcutaneously administering the reconstituted composition to the subject. The disclosure further provides a pharmaceutical composition which is a reconstituted composition resulting from the lyophilized formulation of the disclosure mixed with a diluent.

The disclosure further provides a process for preparing a lyophilized composition. The process comprises the following steps: (a) freezing the pharmaceutical composition disclosed herein; (b) annealing the pharmaceutical composition of step (a) at a temperature of about −5° C. to about −15° C.; (c) primary drying the product of step (b); and (d) secondary drying the product of step (c). Remarkably, the process disclosed herein produces a lyophilized EFX drug product with enhanced properties. For example, the resulting product of the lyophilization process can be reconstituted in a remarkably short time (ranging from less than 1 minute to up to 10 minutes) compared to the product of other lyophilization conditions. In many cases, reconstitution time is improved by approximately 50% or more compared to other pharmaceutical compositions and lyophilization processes. Further, the process for preparing a lyophilized composition disclosed herein significantly decreases the specific surface area (less dense cakes) of the resulting cake which is associated with significantly shorter reconstitution times. This provides a significant advantage to clinicians and patients, as reconstitution can be performed shortly before administration, minimizing time for preparation of dose at the point of care or prior to self-administration.

In various aspects, the freezing in step (a) of the lyophilization process is conducted at a temperature of between about −40° C. to about −50° C. In various aspects, the freezing in step (a) is conducted at a temperature of about −40° C., about −41° C., about −42° C., about −43° C., about −44° C., about −45° C., about −46° C., about −47° C., about −48° C., about −49° C., or about −50° C.

In various aspects, the annealing in step (b) is conducted for about 5 hours to about 20 hours. In various aspects, the annealing in step (b) is conducted for about 5 hours, about 6 hours, about 7 hours, about 8 hours, about 9 hours, or about 10 hours.

In various aspects, the annealing in step (b) is conducted at a temperature of between about −5° C. to about −20° C. In various aspects, the annealing in step (b) is conducted at a temperature of about −5° C., about −6° C., about −7° C., about −8° C., about −9° C., about −10° C., about −11° C., about −12° C., about −13° C., about −14° C., about −15° C., about −16° C., about-17° C., about −18° C., about −19° C., or about −20° C.

In various aspects, the primary drying in step (c) is conducted at about 0.08 to 0.2 mbar chamber pressure. In various aspects, the primary drying in step (c) is conducted at about 0.08, about 0.09, about 0.10, about 0.11, about 0.12, about 0.13, about 0.14, about 0.15, about 0.16, about 0.17, about 0.18, about 0.19, or about 0.20 mbar chamber pressure.

In various aspects, the primary drying in step (c) is conducted at a temperature from about −5° C. to −30° C. In various aspects, the primary drying in step (c) is conducted at a temperature of about −10° C., about −11° C., about −12° C., about −13° C., about −14° C., about −15° C., about −16° C., about −17° C., about −18° C., about −19° C., about −20° C., about −21° C., about −22° C., about −23° C., about −24° C., about −25° C., about −26° C., about −27° C., about −28° C., about −29° C., or about −30° C.

In various aspects, the secondary drying in step (d) is conducted at a temperature from about 35 to 55° C. In various aspects, the secondary drying in step (d) is conducted at a temperature of about 35° C., about 40° C., about 45° C., about 50° C., or about 55° C.

The EFX pharmaceutical composition disclosed herein can be used to treat, ameliorate, prevent or reverse a number of diseases, disorders, or conditions, including, but not limited to metabolic disorders. In various aspects, the disclosure provides a method of treating a disease or disorder, wherein the method comprises administering the pharmaceutical composition comprising EFX to a subject (e.g., a human) in need thereof. The disease or disorder may be any following: non-alcoholic steatohepatitis (NASH), nonalcoholic fatty liver disease (NAFL), hepatic steatosis, alcoholic steatohepatitis (ASH), alcoholic liver disease (ALD) or alcoholic fatty liver disease (AFL), diabetes (e.g., type 2 diabetes), obesity, cravings or addictions (alcohol-related or other, such as food), hypertriglyceridemia, dyslipidemia, cardiovascular disease (such as atherosclerosis), or aging. In various aspects, the disclosure provides a method of reversing liver cirrhosis or reducing fibrosis associated with NASH, ASH, ALD, AFL, or protein misfolding disease. In this respect, following treatment, the subject's fibrosis score, based on NASH Clinical Research Network (CRN) histological scoring system, (Kleiner D et al, 2005 Hepatology 41, 1313), preferably regresses from F4 (cirrhosis) to F3 (advanced fibrosis) or less. In exemplary embodiments, addiction encompasses persistent, compulsive dependence on a behavior or substance such as alcohol, drugs, or nicotine. In exemplary embodiments, craving encompasses a strong, urgent, or abnormal desire for a certain substance or activity, such as sugar. The disclosure also provides a method of normalizing liver fat content, reducing blood glucose levels, increasing insulin sensitivity, and/or reducing uric acid levels, by administering the pharmaceutical EFX composition disclosed herein to a subject in need thereof. In exemplary embodiments, normalizing liver fat content refers to reducing liver fat content (e.g., absolute liver fat content), preferably reducing liver fat content to that of a typical, healthy, non-diseased subject (i.e. a subject not suffering from one or more of the diseases/disorders described herein). In various embodiments, liver fat content is reduced to <5% absolute liver fat. An absolute liver fat content of ≥5% is associated with hepatic steatosis (fatty liver disease), with <5% absolute liver fat content being considered within a clinically normal range for non-diseased subjects (see, for example, Chalasani et al., 2018 Hepatology; 67(1):328-357).

In a phase 2 clinical trial as a treatment for Non-Alcoholic Steatohepatitis (NASH), Efruxifermin has shown unprecedented levels of efficacy, including normalization of liver fat and regression of fibrosis in approximately half of the patients after only 16 weeks of dosing. The uniqueness of EFX's clinical profile is underlined by also restoring a healthy lipoprotein profile, improving glycemic control (reduced hemoglobin A1c by 0.6-0.9% among type 2 diabetic NASH patients), and reducing uric acid levels (Harrison et al., 2021, Nat Medicine 27:1262-1271).

A “subject in need thereof” is a subject, such as a human, that would benefit from the administration of the pharmaceutical composition, and may be diagnosed with or suffering from symptoms of any of the disorders described herein. For example, the subject in need of reducing uric acid levels may be a subject suffering from gout. The subject in need of a method of reversing liver cirrhosis or reducing fibrosis associated with NASH, ASH, ALD, ALF, or protein misfolding disease may be suffering from NASH, ASH, ALD, ALF, or protein misfolding disease, or recovering from NASH, ASH, ALD, ALF, or protein misfolding disease.

In various aspects, the disorder is a protein misfolding disease. Misfolded proteins can trigger a variety of pathogenic responses, and are believed to be responsible for, or at least associated with, a number of human diseases. Exemplary protein misfolding diseases include, but are not limited to, cystic fibrosis, alpha-1 antitrypsin deficiency, and transthyretin amyloid cardiomyopathy. The method comprises administering to a subject in need thereof a pharmaceutical composition of the disclosure in an amount effective to achieve a desired biological response. In related aspects, the method comprises administering the pharmaceutical EFX composition as part of a therapeutic regimen which also includes administration of a misfolded protein corrector molecule or an oligonucleotide-based therapeutic for a protein misfolding disease (such as alpha-1 antitrypsin deficiency).

The term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease or disorder can provide any amount or any level of treatment. Furthermore, the treatment provided by the method may include treatment of one or more conditions or symptoms or signs of the disease being treated and/or improving quality of life of the subject with the condition or disease. The treatment method of the present disclosure may inhibit one or more symptoms of the disease. Also, the treatment provided by the methods of the present disclosure may encompass slowing or reversing progression of the disease.

Improvement of quality of life of a subject can be measured by determining one or more quality of life parameters using, for instance, the European Quality of Life 5 questions tool (EQ-5D) to determine mobility, mood, holistic impact on patients' quality of life as reported by patients. The EQ-5D questionnaire also includes a Visual Analog Scale (VAS), by which respondents can report their perceived health status. See, for example, Balestroni et al., Monaldi Arch Chest Dis. 2012 September; 78(3):155-9, which is incorporated by reference in its entirety. Treatment may also be monitored using a Liver Disease Questionnaire. See, for example, Younossi et al., Clin Gastroenterol Hepatol. 2019 September; 17(10):2093-2100.e3, which is incorporated by reference in its entirety. Liver treatment also may be monitored by measuring objective parameters such as histology data (e.g., regression of fibrosis, resolution of NASH, and the like) and biomarkers of liver injury (e.g., alanine aminotransferase (ALT), aspartate transaminase (AST), gamma-glutamyl transferase (GGT), and/or alkaline phosphatase (ALP)). Exemplary methods of histopathology scoring of liver in NASH patients are disclosed in, for example, Kleiner et al, 2005 Hepatology 41, 1313, which is incorporated by reference in its entirety.

With regard to the foregoing methods, the composition may be administered by any suitable route of administration, including intravenous, intraperitoneal, intracerebral (intra-parenchymal), intramuscular, intra-ocular, intraarterial, intraportal, intramedullary, intrathecal, intraventricular, intradermal, transdermal, subcutaneous, intranasal, inhalation (e.g., upper and/or lower airways), enteral, epidural, urethral, vaginal, or rectal routes of administration. In various instances, the composition is administered to the subject intravenously, intramuscularly, or subcutaneously. For example, in some aspects, the composition is administered subcutaneously. The amount or dose of EFX in the composition (i.e., the “effective amount”) administered should be sufficient to achieve a desired biological effect in the subject over a clinically reasonable time frame.

In jurisdictions that forbid the patenting of methods that are practiced on the human body, the meaning of “administering” a composition to a human subject may be restricted to prescribing a controlled substance that a human subject can self-administer by any technique (e.g., injection, insertion, etc.). The disclosure contemplates use of the pharmaceutical composition to treat any of the diseases or disorders described here. The disclosure further contemplates use of the composition in the preparation of a medicament for treating any of the diseases or disorders described herein. The disclosure further provides the composition described herein for use in the treatment of any of the diseases or disorders referenced here. In jurisdictions that do not forbid the patenting of methods that are practiced on the human body, the “administering” of compositions includes both methods practiced on the human body and also the foregoing activities.

As an additional aspect, kits are provided which comprise a pharmaceutical composition described herein packaged in a manner which facilitates administration to subjects. In one aspect, the kit includes a pharmaceutical composition/formulation described herein packaged in a container such as a sealed bottle, vessel, single-use or multi-use vial, prefilled device (e.g. syringe), or prefilled injection device, optionally with a label affixed to the container or included in the package that describes use of the pharmaceutical composition in practicing the method. In one aspect, the pharmaceutical composition is packaged in a unit dosage form. The kit may include a device suitable for administering the pharmaceutical composition according to a specific route of administration, although this is not required. For example, the disclosure provides a dual chamber device for delivering the pharmaceutical composition disclosed herein to a subject in need thereof. Dual chamber devices are combination products containing the lyophilized pharmaceutical composition disclosed herein and a diluent in two separate chambers of the device. Prefilled dual chamber devices (DCDs) are combination products containing freeze-dried drug and diluent in two separate chambers of the device. DCDs provide high stability and convenience to patients and doctors, thus significantly improving product quality, patient compliance, and market competitiveness. DCDs also provide seal integrity, sterility and compatibility with biopharmaceuticals and avoid leachability and needle stick injuries. Suitable dual chamber devices for use with the instant disclosure are described in the art. See for example, Ingle R., Fang W. (2021). Int. Journal of Pharmaceutics 597, 12031.

The Examples below illustrate representative features of the disclosure. From the description of these aspects, other aspects of the invention can be made and/or practiced based on the description provided below. The methods involve use of molecular biological techniques described in treatises such as Molecular Cloning: A Laboratory Manual, 2nd ed., vol. 1-3, Sambrook et al., ed., Cold Spring Harbor Laboratory Press, Cold Spring Harbor, N.Y., 2001; and Current Protocols in Molecular Biology, Ausubel et al., ed., Greene Publishing and Wiley-Interscience, New York.

### EXAMPLES

### Example 1: Efruxifermin Formulations

This example describes EFX formulations evaluated in the studies described below.

Parental injectable protein-based biologics are frequently formulated at slightly acidic to neutral pH in the range of pH 5.2 to approximately pH 6.9, to minimize posttranslational modifications, such as deamidation or oxidation, which converts asparagine residues in a protein to aspartic acid or isoaspartic acid as the intermediate succinimide, and glutamine to glutamic acid or pyroglutamic acid. Above pH 7.6, deamidation (acidic charge variants formation) is frequently observed, resulting in lost stability, functionality, and/or protein potency. Below pH 7.0, basic charged variants formation is also observed.

Seeking to minimize deamidation, oxidation, and formation of charge variants, EFX formulations were developed with a variety of excipients in the pH range 4.5-7.0. Surprisingly, the viscoelastic properties of EFX changed dramatically at pH below 6.5, manifesting gel-like behavior, phase separation, and loss of fluidity. These features are challenging for an injectable formulation of a biologic. The high dynamic viscosity of these gels (in some instances as much as 23,950 cP (centipoise)), as well as the prolonged stability of the gels (in some cases for as long as 21 months stored at 2-8° C.), indicate formation of an ordered, stable three-dimensional structure, possibly as a result of cross-linked hydrogen bonds, arrayed in a pattern-forming lattice.

In addition, a majority of the tested formulations at or below pH 6.9 demonstrated a propensity for protein aggregation, clipping/fragmentation, and/or formation of visible and subvisible particles. Such changes are undesirable for injectable biologics, since they may be associated with safety (particularly immunogenicity), instability and loss of potency concerns.

Thus, a series of studies were designed to develop an EFX formulation, focusing on minimizing degradation pathways and posttranslational modifications (such as increased formation of charge variants), while also overcoming the unique propensity of EFX to form cross-linked lattices of hydrogen bonds, resulting in gel formation and significant phase separation.

EFX formulations which were evaluated in Example 1 and the subsequent examples, are listed in Table 1, and the excipients and chemicals used are listed in Table 2.

TABLE 1
EFX formulations evaluated*.

| | | EFX |
|---|---|---|
| | | concentration |
| Formulation | Formulation composition | range mg/mL |
| F1 | 20 mM Na succinate/succinic acid, 220 mM trehalose, PS20, pH 4.5 | Up to 100 ± 10 |
| F2 | 20 mM Na succinate/succinic acid, 160 mM Lys-HCl, PS20, pH 5.0 | Up to 100 ± 10 |
| F3 | 20 mM Na glutamate/glutamic acid, 220 mM trehalose, PS20, pH 5.0 | Up to 100 ± 10 |
| F4 | 20 mM Na glutamate/glutamic acid, 200 mM trehalose, 40 mM Lys-HCl, | Up to 100 ± 10 |
| | PS20, pH 5.0 | |
| F5 | 20 mM Na acetate/acetic acid, 220 mM trehalose, PS20, pH 5.2 | Up to 100 ± 10 |
| F6 | 20 mM Na acetate/acetic acid, 220 mM sucrose, PS20, pH 5.2 | Up to 100 ± 10 |
| F7 | 20 mM Na succinate/succinic acid, 220 mM trehalose, PS20, pH 5.2 | Up to 100 ± 10 |
| F8 | 20 mM Na succinate/succinic acid, 120 mM NaCl, PS20, pH 5.2 | Up to 100 ± 10 |
| F9 | 20 mM Na succinate/succinic acid, 220 mM sucrose, PS20, pH 5.5 | Up to 100 ± 10 |
| F10 | 20 mM glycylglycine/glycylglycine-HCl, 220 mM trehalose, PS20, pH 5.5 | Up to 100 ± 10 |
| F11 | 20 mM Histidine/His-HCl, 220 mM Trehalose, PS20, pH 6.0 | Up to 100 ± 10 |
| F12 | 20 mM His/His-HCl, 180 mM trehalose, 40 mM Lys-HCl, PS20, pH 6.5 | Up to 100 ± 10 |
| F13 | 20 mM His/His-HCl, 220 mM sucrose, PS20, pH 7.0 | Up to 100 ± 10 |
| F14 | 20 mM Na phosphate, 190 mM sucrose, 40 mM Met, PS20, pH 7.5 | Up to 100 ± 10 |
| F15 | 20 mM Na phosphate, 190 mM trehalose, 40 mM Lys-HCl, PS20, pH 7.5 | Up to 100 ± 10 |
| F16 | 20 mM Na phosphate, 180 mM sucrose, 40 mM Glu, 40 mM Arg, PS20, | Up to 100 ± 10 |
| | pH 7.5 | |
| F17 | 180 mM sucrose, 40 mM Arg/Arg-HCl, PS20, pH 7.8 | 28 to 100 ± 10 |
| F18 | 20 mM Tris/Tris-HCl, 160 mM Lys-HCl, PS20, pH 7.8 | 28 to 150 ± 15 |
| F19 | 20 mM Tris/Tris-HCl, 220 mM sucrose, PS20, pH 8.3 | Up to 100 ± 10 |
| F20 | 20 mM Tris/Tris-HCl, 220 mM sucrose, PS20, pH 9.0 | Up to 100 ± 10 |
| F21 | 20 mM Histidine, 220 sucrose, 20 mM Arg/Arg-HCl, PS20, pH 6.5 | Up to 100 ± 10 |
| F22 | 20 mM Tris, 220 sucrose, 20 mM Arg/Arg-HCl, PS20, pH 7.5 | Up to 100 ± 10 |
| F23 | 20 mM Histidine, 220 sucrose, 20 mM Arg/Glutamate, PS20, pH 6.5 | Up to 100 ± 10 |
| F24 | 20 mM Tris, 220 sucrose, 20 mM Arg/Glutamate, PS20, pH 7.5 | Up to 100 ± 10 |
| F25 | 20 mM Histidine, 100 sucrose, 140 mM Arg/Arg-HCl, PS20, pH 6.5 | Up to 100 ± 10 |
| F26 | 20 mM Tris, 100 sucrose, 140 mM Arg/Arg-HCl, PS20, pH 7.5 | Up to 100 ± 10 |
| F27 | 20 mM Histidine, 100 sucrose, 140 mM Arg/Glutamate, PS20, pH 6.5 | Up to 100 ± 10 |
| F28 | 20 mM Tris, 100 sucrose, 140 mM Arg/Glutamate, PS20, pH 7.5 | Up to 100 ± 10 |
| F29 | 20 mM Histidine, 160 sucrose, 80 mM Arg/Glutamate, PS20, pH 7.0 | Up to 100 ± 10 |
| F30 | 20 mM Tris, 160 sucrose, 80 mM Arg/Glutamate, PS20, pH 7.5 | 50 to 100 ± 10 |
| F31 | 20 mM Tris-HCl, 120 mM Arg/Arg-HCl, 120 mM sucrose, 0.02% w/v | Up to 100 ± 10 |
| | PS20, pH 7.3 | |
| F32 | 20 mM Tris-HCl, 120 mM Arg/Arg-HCl, 120 mM sucrose, 0.02% w/v | Up to 100 ± 10 |
| | PS80, pH 7.3 | |
| F33 | 20 mM Tris-HCl, 120 mM Arg/Arg-HCl, 120 mM sucrose, 0.06% w/v | Up to 150 ± 15 |
| | PS20, pH 7.3 | |
| F34 | 20 mM Tris-HCl, 120 mM Arg/Arg-HCl, 120 mM sucrose, 0.06% w/v | Up to 100 ± 10 |
| | PS80, pH 7.3 | |
| F35 | 20 mM Tris-HCl, 120 mM Arg/Arg-HCl, 120 mM sucrose, 0.1% w/v PS20, | Up to 100 ± 10 |
| | pH 7.3 | |
| F36 | 20 mM Tris-HCl, 120 mM Arg/Arg-HCl, 120 mM sucrose, 0.1% w/v PS80, | Up to 100 ± 10 |
| | pH 7.3 | |
| F37 | 120 mM Arg/Arg-HCl, 120 mM sucrose 0.06% w/v PS20, pH 7.3 | Up to 100 ± 10 |
| F38 | 120 mM Arg/Arg-HCl, 120 mM sucrose, 0.06% w/v PS80, pH 7.3 | Up to 100 ± 10 |
| F39 | 120 mM Arg/Arg-HCl, 120 mM sucrose, 0.06% w/v PS80, pH 6.9 | Up to 100 ± 10 |
| F40 | 120 mM Arg/Arg-HCl, 120 mM sucrose, 0.06% w/v PS80, pH 7.7 | Up to 100 ± 10 |
| F41 | 20 mM Tris-HCl, 120 mM Arg/Arg-HCl, 120 mM sucrose, pH 7.3 | Up to 100 ± 10 |
| F42 | 40 mM Arg/Arg-HCl, 180 mM sucrose, 0.04% w/v PS20, pH 7.3 | Up to 100 ± 10 |

*Formulation Designation (F1-F20 excipients/pH screening study; F21-F30 Arg/Glu/pH study of conformational stability as well as minimizing aggregation and charge variants formation; F31-F42 study of surfactants). Polysorbate 20 concentration was 0.04% w/v unless stated in the table. All amino-acids listed are natural state (i.e., L-isomers).

TABLE 2
Overview of excipients and chemicals
used in EFX formulation development.
Material

L-Histidine, excipient Ph. Eur., USP, JP
Succinic acid
Tris base, ≥99.9%, EP, USP
Tris-HCl, Emprove Expert
D(+) sucrose, USP-NF, BP, Ph. Eur., JP
α,α-Trehalose Dihydrate, High Purity (Low Endotoxin),
USP/NF, EP, JP, ChP, 25 kg
Polysorbate 20
L-arginine base, Ph. Eur., USP
L-glutamic acid, Ph. Eur., USP
Hydrochloric acid 1N
Hydrochloric acid 2N
Sodium dihydrogenphosphate dihydrate, Ph. Eur., USP
Di-Sodium hydrogenphosphate Dodecahydrate, Ph. Eur., USP
Sodium chloride; Ph. Eur., USP
Sodium hydroxide solution 2N
Sodium hydroxide solution 1N
L(+)-lysine monohydrochloride
L-methionine, Ph. Eur., USP
Acetic acid, glacial, Ph. Eur., USP
Glycylglycine
Potassium Dihydrogenphosphate, USP
Potassium Phosphate Anhydride, USP
Sodium Citrate, USP, EP, JP
Na-carboxy methyl cellulose
PEG-4000

### Example 2: Characterizing Formulations: Gel Formation and Phase Separation (Schlieren Phase Separation)

This Example describes physical properties of various formulations described herein. Surprisingly, EFX has a propensity to form gels and to phase separate when formulated and stored under conditions which are suitable for most other biologics.

### Visual Appearance

EFX formulations were inspected for gel formation, phase separation, opalescence, and the presence or absence of visible particles under gentle, manual, radial agitation for 5 seconds in front of white background and for 5 seconds in front of black background according to the European Pharmacopoeia (9th edition; monograph 2.9.20) at 2,000-3,750 lux. To classify the observed visible particles, a numerical score based on the “Deutscher Arzneimittel-Codex” (DAC 2006) was applied, as listed in Table 3. Fiber-like structures, particles that are likely non-inherent, and additional sample attributes were documented as described in Table 4.

TABLE 3
Numerical score for visible particles, excluding
fibers and particles that are non-inherent.

| Score for visible | |
|---|---|
| particles | Description |
| 0 | No particles visible within 5 sec |
| 1 | Few particles visible within 5 sec |
| 2 | Medium number of particles visible within 5 sec |
| 10 | Large number of particles directly visible |

TABLE 4
Letter code for additional visual appearance and observations.

| Letter | Description |
|---|---|
| A | Air bubbles |
| C | Color |
| F | Fiber (single) |
| FF | Multiple fibers (more than one) |
| H | Hurricane, tornado, e.g., because of sedimentation or floating |
| | particles |
| L | Particles that are on the limit of being visible as distinct particles |
| S | Schlieren*, phase separation |
| T | Turbidity, opalescence, cloudiness, haziness |
| V | Viscosity |
| X | Non-inherent particles: metal, glitter, rubber parts, glass |
| | delamination |

*Schlieren are optical heterogeneities that can be observed in media with an inhomogeneous refractive index, e.g., liquid media comprising different liquids or liquid phases that exhibit differences in density. For example, Schlieren can be caused by insufficient mixing or by phase separation.

### Protein Concentration (Phase Separation, Schlieren Phase Separation)

EFX concentration was determined by UV/visible spectroscopy using slope spectroscopy with a SoloVPE instrument measuring absorbance at 280 nm.

### Viscosity (Gel Formation)

Dynamic viscosity was measured by using a Kinexus ultra plus rheometer (Malvern Instruments). The rheometer was equipped with a cone-plate setup (cone diameter 40 mm, 1° angle). The dimensions of the measurement CP1/40 cone fixed the measurement gap to 0.024 mm, which required a sample volume of ˜310 μl. To avoid drying of the sample surface, an evaporation blocker was applied. Measurements were conducted at a constant shear rate of 400 s⁻¹ at 25° C. for a period of 3 minutes. In addition, 11 data point tables were generated with rising shear rates from 10 s⁻¹ to 1000 s⁻¹ at 25° C.

### Hydrogen-Deuterium (H-D) Exchange Guanidinium/Urea Mass Spectrometry

The gel formation and phase separation of EFX in formulations F1, F2, F4, F7, F8, F9, and F11 is a result of cross-linked hydrogen bonds, adopting a pattern-forming lattice. Studies with H-D exchange elucidate the mechanism of lattice formation and Schlieren phase separation.

### Data Summary: Gel Formation and Phase Separation (Schlieren Phase Separation)

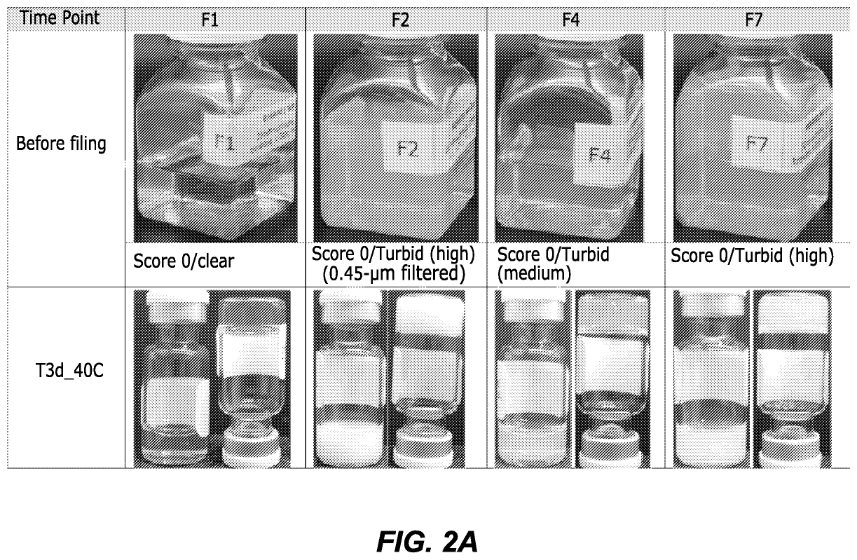

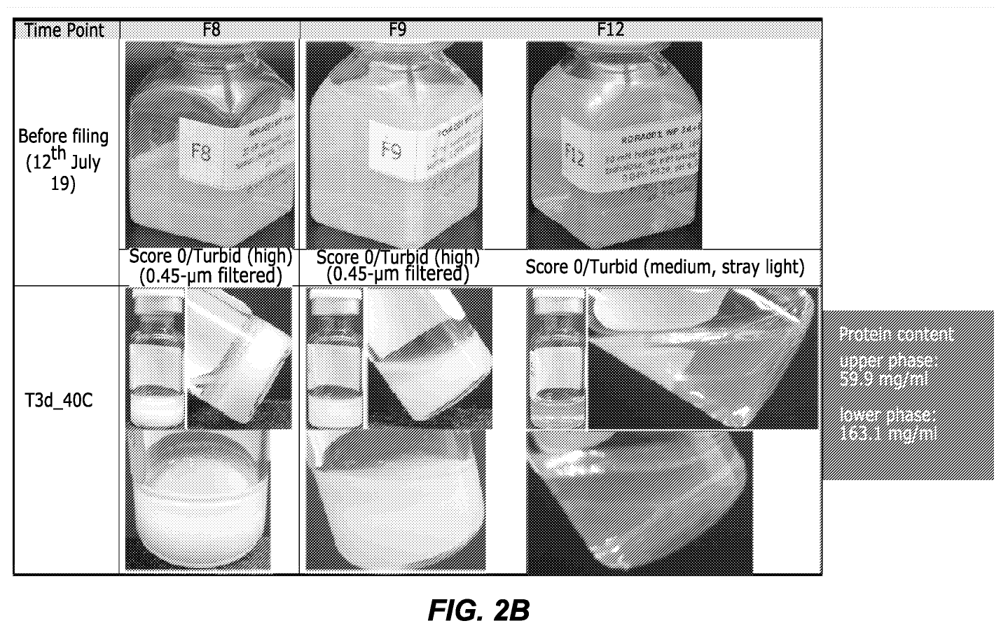

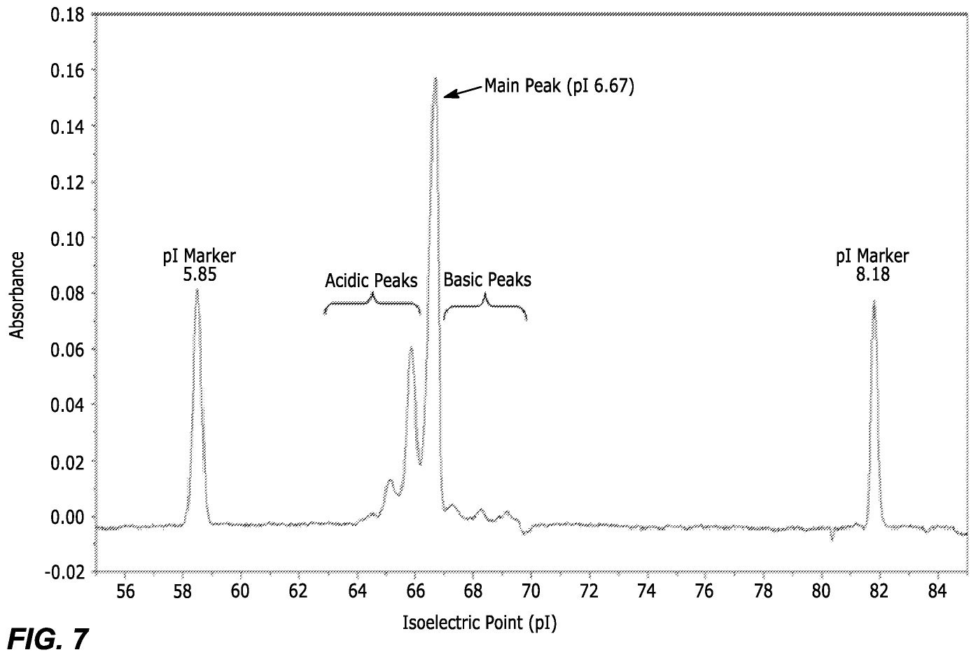

The isolectric point (pI) of EFX, theoretical and measured by icIEF, is approximately pH 6.6. Formulation F1-F12 were formulated at a pH below the pl, whereas the pH values of formulations F13-F20 were above the pl (see Table 1). EFX in formulations compounded below pH 6.5 (low to neutral pH formulations) showed a propensity to undergo gelation or phase separation. Images of gel formed EFX formulations are shown in FIG. 2. Formulation F11 formed dense 3-D structured gel shortly after compounding. After storage at 40° C./75% RH for three days, formulations F1, F2, F4, F7, F8, and F9 also demonstrated gel formation, phase separation, and/or precipitation. A translucent gel was formed in formulations F1, F2, F4, and F7, manifesting high turbidity within the gel (FIG. 7).

Formulations F8, F9, and F12 appeared phase separated (Schlieren phase separation) with a white gel-like phase at the bottom and a turbid, liquid supernatant on top, observed after 3 days at 40° C./75% RH. As an example, the protein concentration measured in the lower phase of F12, assessed by SoloVPE, was 163.1 mg/mL and in the upper supernatant phase 59.9 mg/mL. Additionally, after two weeks storage at 40° C./75% RH, formulations F5 and F6 showed similar dramatic changes of their visual appearance and viscoelastic properties.

At 25° C./60% relative humidity, formulations F2 and F8 formed dense gels respectively after 1 week and 1 month storage. In addition, phase separation and visible particle formation were observed for F14 after one month of storage at 25° C./60% RH, but not for other temperature conditions (2-8° C. and 40° C./75% RH).

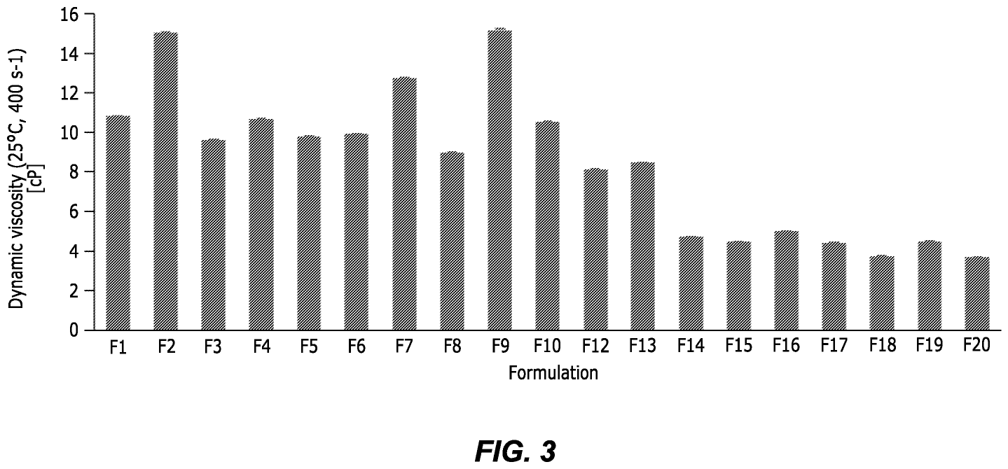

The dynamic viscosities of EFX at 100 mg/mL in formulations F1 to F20 (except F11) at 400 s⁻¹ and time zero are presented in FIG. 3. Measured dynamic viscosities appeared to be pH dependent, increasing at lower pH. For example, viscosities of EFX formulations F14 to F20 at 100 mg/mL and room temperature, all at pH 7.0 and above, were ≤5 cP. By comparison, the measured dynamic viscosities were in the range 10 to 16 cP immediately following compounding of formulations F1 to F10 at pH 6.5 and below (FIG. 3). In formulation 11 (F11), EFX instantly formed a dense/rigid gel lattice on compounding, making further experimental evaluation of dynamic viscosity impossible.

During storage of EFX under various conditions, other formulations at or below pH 6.5 underwent phase separation and formed highly-viscous gels (F1, F2, F4, F7, F8, F9, F10, and F11). The gel formation appeared to be a result of cross-linked hydrogen bonds, shaping a pattern-forming lattice, resulting in highly rigid three-dimensional structure. The rate of gel EFX formation depended on storage temperature, occurring within approximately 3 days at 40° C., but after weeks or months at 25° C. and 2-8° C. respectively. Once formed, the three-dimensional gel lattices appeared stable or irreversible, as evidenced by formulations F1, F2, F4, F7, F8, F9, and F11 retaining their gel-like appearance after 21 months under various storage conditions.

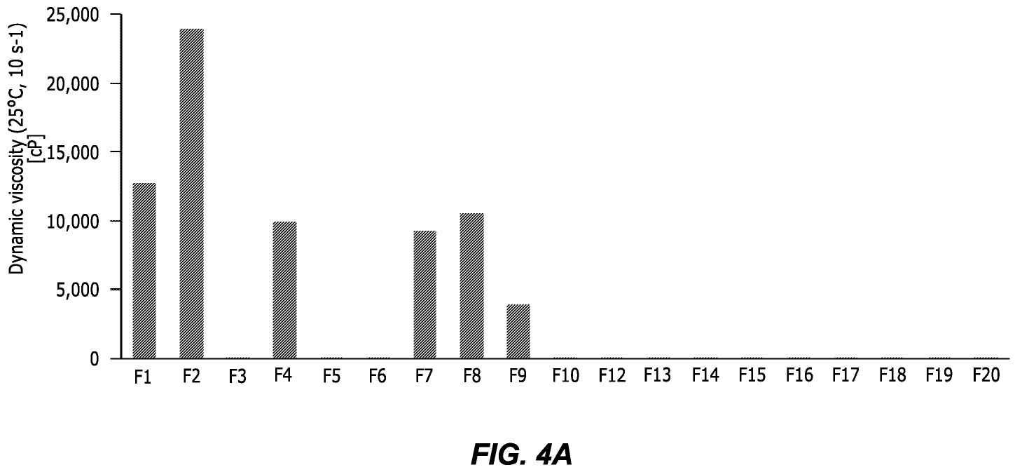

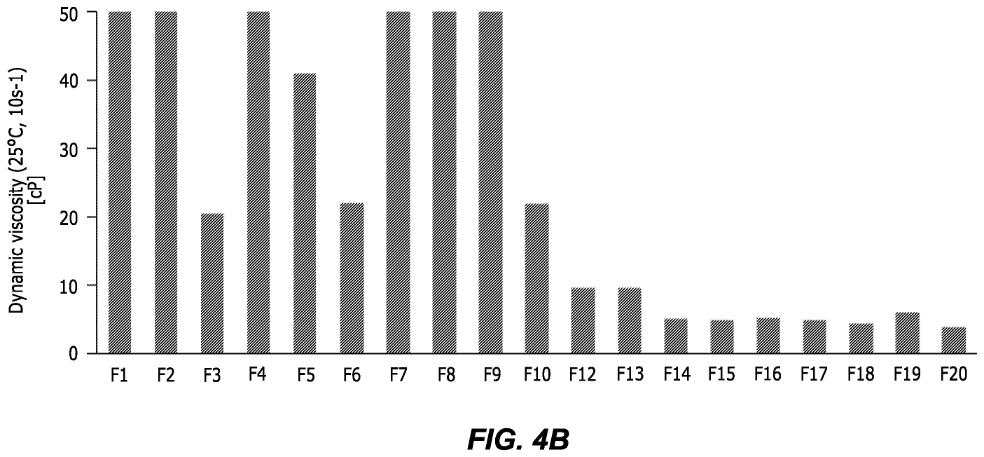

FIG. 4 shows the dynamic viscosities of EFX at 100 mg/mL for F1 to F20 (except F11) at 10 s⁻¹ shear rate after 3 days at 40° C. Those formulations which formed gels appear to have dynamic viscosity in tens of thousands of cP. The dynamic viscosities of EFX remained unchanged after storage for 21 months at 2-8° C. (FIG. 4 and Table 5). For example, the measured dynamic viscosities of F2 and F10 at low shear rate were as high as 23,950 cP and 10,560 cP, comparable to pourable silicone rubber and chocolate syrup, respectively.

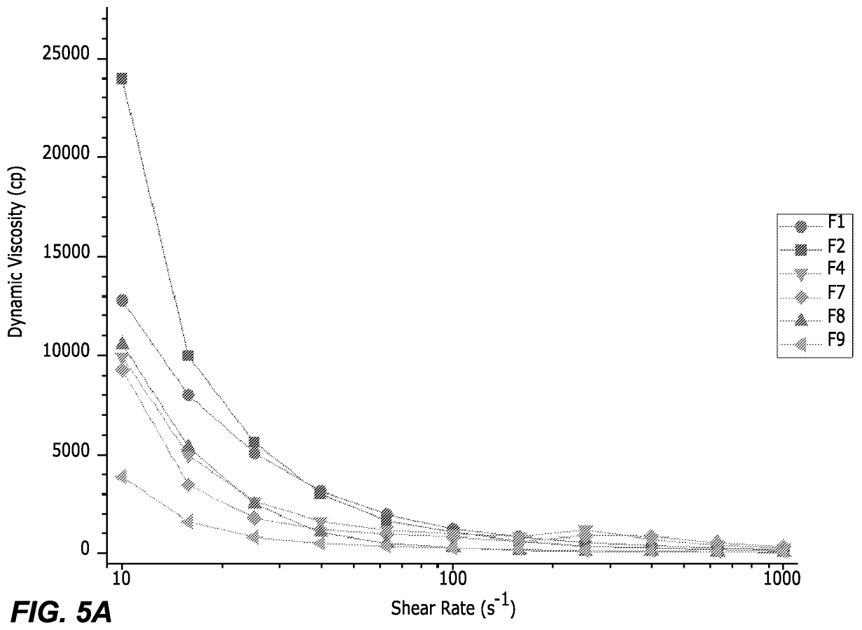

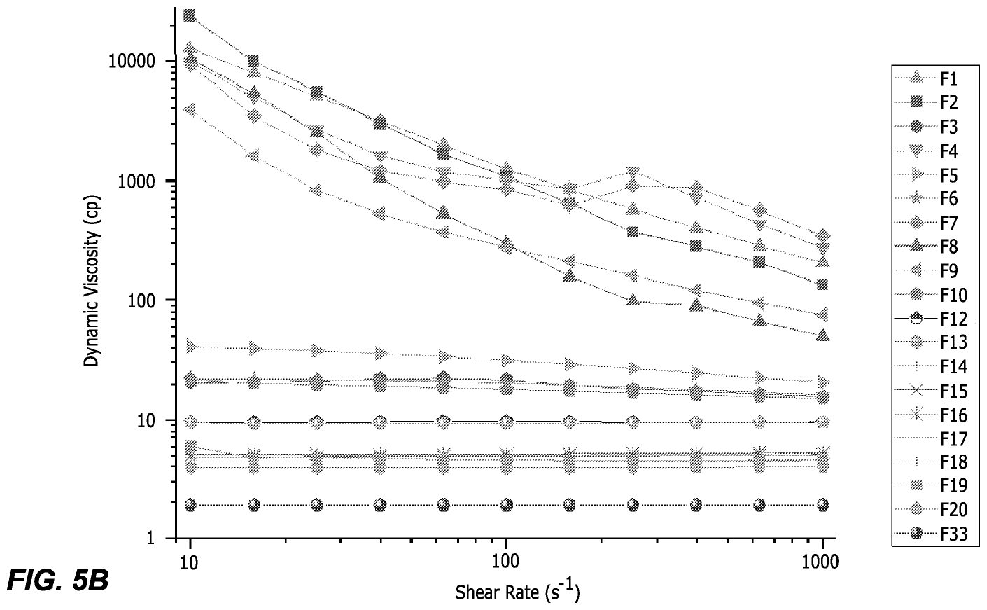

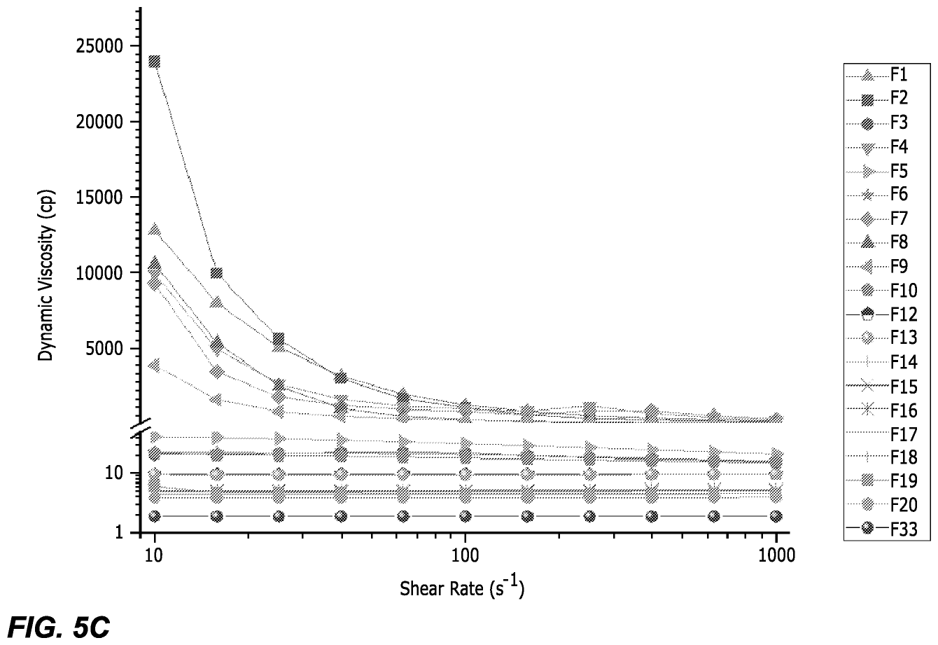

Formulations F1, F2, F4, F7, F8, F9, and F11 formed irreversible gels and underwent phase separation, demonstrating distinctly different viscoelastic properties as a function of shear rates, compared to homogenous liquid formulations such as F13 to F42. As illustrated in FIG. 5, the dynamic viscosities of EFX in formulations F13 to F20, as well as F21 to F42 (not shown), as a function of increasing shear rates remained constant, thus demonstrating Newtonian behavior. In contrast, the viscosity of F1, F2, F4, F7, F8, F9, and F11 appeared to decrease as a function of increasing shear rate, thus manifesting non-Newtonian shear thinning effect. Despite this, they remained gel-like indicating the hydrogen bond linked lattices are sufficiently stable to withstand high shear rates (such as 1000 s⁻¹). In addition to pH dependency, gel formation and phase separation also appeared to depend on the excipients comprising each formulation. Illustrating this point, different compositions of excipients but similar pH for F1, F2, F4, F7, F8, F9, and F11 were associated with a wide range of dynamic viscosities. Such differences were observed both at low and high shear rates, thus demonstrating that the viscoelastic properties exhibit strong dependence on the nature of the specific excipients. Dynamic viscosities at low shear rate are summarized in Table 5A and at 1000 s⁻¹ shear rate in Table 5B.

TABLE 5A
Dynamic viscosity of 100 mg/mL EFX in F1-F20 (except F11) formulations at 10
s⁻¹ shear rate after 3 days at 40° C. followed by storage for 21 months at 2-8° C.

| | | Viscosity |
|---|---|---|
| Formulation | Formulation composition | [cP] |
| F1 | 20 mM Na succinate/succinic acid, 220 mM trehalose, PS20, pH 4.5 | 12 770 |
| F2 | 20 mM Na succinate/succinic acid, 160 mM Lys-HCl, PS20, pH 5.0 | 23 950 |
| F3 | 20 mM Na glutamate/glutamic acid, 220 mM trehalose, PS20, pH 5.0 | 21 |
| F4 | 20 mM Na glutamate/glutamic acid, 200 mM trehalose, 40 mM Lys-HCl, | 9 920 |
| | PS20, pH 5.0 | |
| F5 | 20 mM Na acetate/acetic acid, 220 mM trehalose, PS20, pH 5.2 | 41 |
| F6 | 20 mM Na acetate/acetic acid, 220 mM sucrose, PS20, pH 5.2 | 22 |
| F7 | 20 mM Na succinate/succinic acid, 220 mM trehalose, PS20, pH 5.2 | 9 279 |
| F8 | 20 mM Na succinate/succinic acid, 120 mM NaCl, PS20, pH 5.2 | 10 560 |
| F9 | 20 mM Na succinate/succinic acid, 220 mM sucrose, PS20, pH 5.5 | 3 878 |
| F10 | 20 mM glycylglycine/glycylglycine-HCl, 220 mM trehalose, PS20, pH 5.5 | 22 |
| F11 | 20 mM Histidine, 220 mM Trehalose, PS20, pH 6.0 | UM* |
| F12 | 20 mM His/His-HCl, 180 mM trehalose, 40 mM Lys-HCl, PS20, pH 6.5 | 10.0 |
| F13 | 20 mM His/His-HCl, 220 mM sucrose, PS20, pH 7.0 | 9.5 |
| F14 | 20 mM Na phosphate, 190 mM sucrose, 40 mM Met, PS20, pH 7.5 | 5.1 |
| F15 | 20 mM Na phosphate, 190 mM trehalose, 40 mM Lys-HCl, PS20, pH 7.5 | 4.9 |
| F16 | 20 mM Na phosphate, 180 mM sucrose, 40 mM Glu, 40 mM Arg, PS20, | 5.1 |
| | pH 7.5 | |
| F17 | 180 mM sucrose, 40 mM Arg/Arg-HCl, PS20, pH 7.8 | 4.9 |
| F18 | 20 mM Tris/Tris-HCl, 160 mM Lys-HCl, PS20, pH 7.8 | 4.4 |
| F19 | 20 mM Tris/Tris-HCl, 220 mM sucrose, PS20, pH 8.3 | 6.0 |
| F20 | 20 mM Tris/Tris-HCl, 220 mM sucrose, PS20, pH 9.0 | 3.9 |
| F33 | 20 mM Tris-HCl, 120 mM Arg/Arg-HCl, 120 mM sucrose 0.06% w/v PS20, | 3.7 |
| | pH 7.3 | |

TABLE 5B
Dynamic viscosity of 100 mg/mL EFX in F1-F20 (except F11) formulations
at 1000 s⁻¹ shear rate after 21 months at 2-8° C.

| | | Viscosity |
|---|---|---|
| Formulation | Formulation composition | [cP] |
| F1 | 20 mM Na succinate/succinic acid, 220 mM trehalose, PS20, pH 4.5 | 206 |
| F2 | 20 mM Na succinate/succinic acid, 160 mM Lys-HCl, PS20, pH 5.0 | 134 |
| F3 | 20 mM Na glutamate/glutamic acid, 220 mM trehalose, PS20, pH 5.0 | 16 |
| F4 | 20 mM Na glutamate/glutamic acid, 200 mM trehalose, 40 mM Lys- | 271 |
| | HCl, PS20, pH 5.0 | |
| F5 | 20 mM Na acetate/acetic acid, 220 mM trehalose, PS20, pH 5.2 | 21 |
| F6 | 20 mM Na acetate/acetic acid, 220 mM sucrose, PS20, pH 5.2 | 16 |
| F7 | 20 mM Na succinate/succinic acid, 220 mM trehalose, PS20, pH 5.2 | 344 |
| F8 | 20 mM Na succinate/succinic acid, 120 mM NaCl, PS20, pH 5.2 | 50 |
| F9 | 20 mM Na succinate/succinic acid, 220 mM sucrose, PS20, pH 5.5 | 76 |
| F10 | 20 mM glycylglycine/glycylglycine-HCl, 220 mM trehalose, PS20, pH | 15 |
| | 5.5 | |
| F11 | 20 mM Histidine, 220 mM Trehalose, PS20, pH 6.0 | UM* |
| F12 | 20 mM His/His-HCl, 180 mM trehalose, 40 mM Lys-HCl, PS20, pH 6.5 | 10.0 |
| F13 | 20 mM His/His-HCl, 220 mM sucrose, PS20, pH 7.0 | 10.0 |
| F14 | 20 mM Na phosphate, 190 mM sucrose, 40 mM Met, PS20, pH 7.5 | 5.0 |
| F15 | 20 mM Na phosphate, 190 mM trehalose, 40 mM Lys-HCl, PS20, pH | 5.0 |
| | 7.5 | |
| F16 | 20 mM Na phosphate, 180 mM sucrose, 40 mM Glu, 40 mM Arg, PS20, | 5.0 |
| | pH 7.5 | |
| F17 | 180 mM sucrose, 40 mM Arg/Arg-HCl, PS20, pH 7.8 | 5.0 |
| F18 | 20 mM Tris/Tris-HCl, 160 mM Lys-HCl, PS20, pH 7.8 | 5.0 |
| F19 | 20 mM Tris/Tris-HCl, 220 mM sucrose, PS20, pH 8.3 | 5.0 |
| F20 | 20 mM Tris/Tris-HCl, 220 mM sucrose, PS20, pH 9.0 | 5.0 |
| F33 | 20 mM Tris-HCl, 120 mM Arg/Arg-HCl, 120 mM sucrose 0.06% w/v | 4.0 |
| | PS20, pH 7.3 | |

Based on their Newtonian behavior as a function of shear rate, very low viscosities, and absence of gel formation or significant Schlieren phase separation, formulations F15, F16, F17, F18, F20, and F33 to F42 (pH 6.5) were selected for further development.

### Example 3: Evaluation of Charge Variants Formation, Protein Aggregation, Clipping/Fragmentation, Cell-Based Potency

In addition to maintaining Newtonian behavior and low solution viscosity, EFX formulations described herein were evaluated with respect to aggregation into Higher Molecular Weight Species (HMWS), clipping or fragmentation into Lower Molecular Weight Species (LMWS), formation of subvisible (SVP) and visible particles, and posttranslational modifications resulting in formation of more acidic or basic charge variants. The rate of formation of charged species is higher for EFX compared to other proteins in general at refrigeration temperatures, and the rate of formation increases at room temperature. The Example describes studies performed seeking to minimize, e.g., charged species formation, HMWS, and the like.

### Posttranslational Modifications: Determination of EFX Charge Variants

Charge heterogeneity of EFX was evaluated by anion exchange chromatography (AEX-HPLC) and imaged capillary isoelectric focusing (icIEF). These two methods separate charge variants of proteins using distinct mechanisms. AEX-HPLC separation is based on exposed charges on a protein's surface interacting with a charged stationary chromatographic matrix. The separation by icIEF is based on each charge variant of a protein migrating electrophoretically to its isoelectric point through a pH gradient established in a separation capillary. As a result of their distinct mechanisms of separation, AEX and icIEF are considered complementary and orthogonal methods for assessing charge heterogeneity of proteins.

### Evaluation of Charge Variants by Anion Exchange Chromatography (AEX-HPLC)

The distribution of charge heterogeneity in EFX formulations was assessed using an anion exchange resin column TSK-Gel Q-STAT (4.6 mm×100 mm, 7 μm particle size) with UV absorbance detection at 230 nm. Negatively charged EFX binds to the positively charged column matrix equilibrated in 20 mM Tris and buffered to pH 8 containing 20% (v/v) acetonitrile. Weakly charged variants of EFX are readily displaced from the chromatographic column by low salt concentration, while the more negatively charged EFX variants require higher salt concentration to displace them. The salt gradient was a linear gradient of 0.7 M Sodium Chloride, pH 8.0 at flow rate of 0.5 mL/min. The chromatographic column temperature was maintained at 30° C. throughout the analysis.

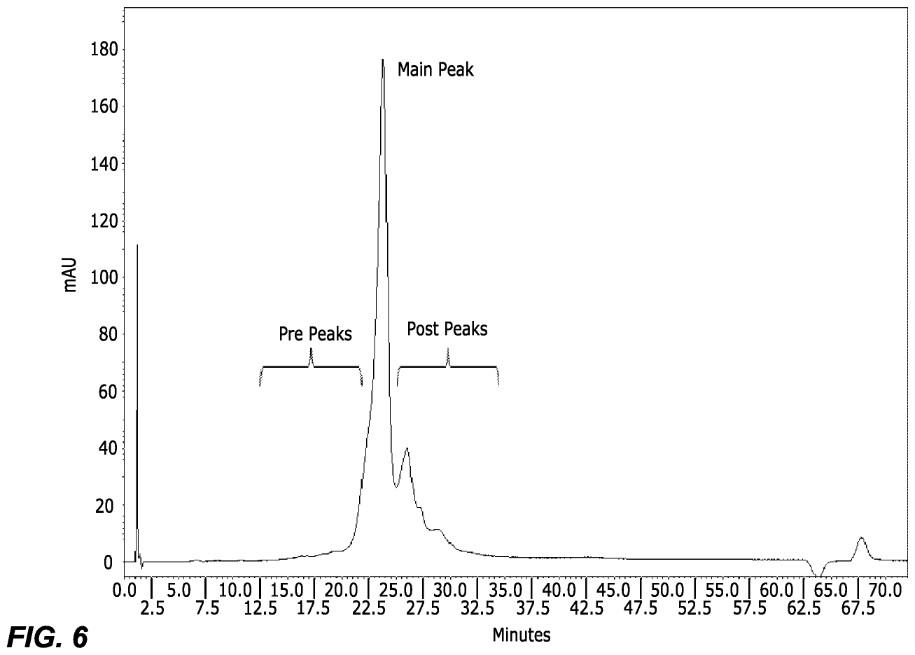

A representative AEX-HPLC chromatogram of EFX formulated in F18 is shown in FIG. 6, with the areas of pre-, post- and main peaks quantitated and summarized in Table 6. For the purpose of chromatographic analysis, the charge variants in EFX are grouped as Pre-peaks (more basic variants, or EFX charge variants with less negative charges on their surface), Main-peak, and Post-peaks (more acidic variants or EFX charge variants with more negative charges on their surface).

TABLE 6
Distribution of Charge Variants in EFX
formulation F18 Separated by AEX-HPLC.

| Peak | Percentage abundance^{±} (%) |
|---|---|
| % Main Peak | 62.0 |
| % Basic Peaks (Pre-peaks) | 8.7 |
| % Acidic Peaks (Post-peaks) | 29.3 |

^{±}Based on total peak area of chromatogram.

Evaluation of Charge Variants by Imaged Capillary Isoelectric Focusing (icIEF)

An imaged capillary isoelectric focusing (icIEF) method was developed using ProteinSimple iCE3 to experimentally confirm the isoelectric point (pI) of EFX. EFX in various formulations was prepared in a mixture containing ampholyte solution, 3 M Urea, and markers corresponding to pl 5.85 and pl 8.18. Separation of charge variants was performed in a coated capillary of 100 μm internal diameter and 50 mm length at ambient temperature, with protein peaks monitored by absorbance at 280 nm. Charge variants were separated using two distinct focusing steps; initially 1,000 V for a minute, followed by 10 minutes of mobilization at 3,000 V.

The main peak of EFX in formulation F18 migrated with an apparent pl of 6.67, which agrees well with the theoretical pl of approximately 6.5 (Error! Reference source not found.). Additional charge variant peaks were detected at low levels in the electropherogram, migrating either before (acidic variants) or after (basic variants) the main peak. Distribution of acidic, main and basic peaks expressed as percentage abundance based on peak-area, is shown in Table. The distribution of pre-peaks, main peak, and post-peaks from the icIEF electropherogram reflects that reported for pre-peaks, main peak, and post-peaks by AEX-HPLC.

TABLE 7
Distribution of Charge Variants in EFX
formulation F18 Separated by icIEF.

| Peak | pI | Percentage abundance^{±} (%) |
|---|---|---|
| % Main Peak | 6.67 | 57.4 |
| % Acidic Peaks | <6.60 | 32.7 |
| % Basic Peaks | >6.70 | 9.8 |

icIEF = imaged capillary isoelectric focusing.
^{±}Based on total peak area of electropherogram.

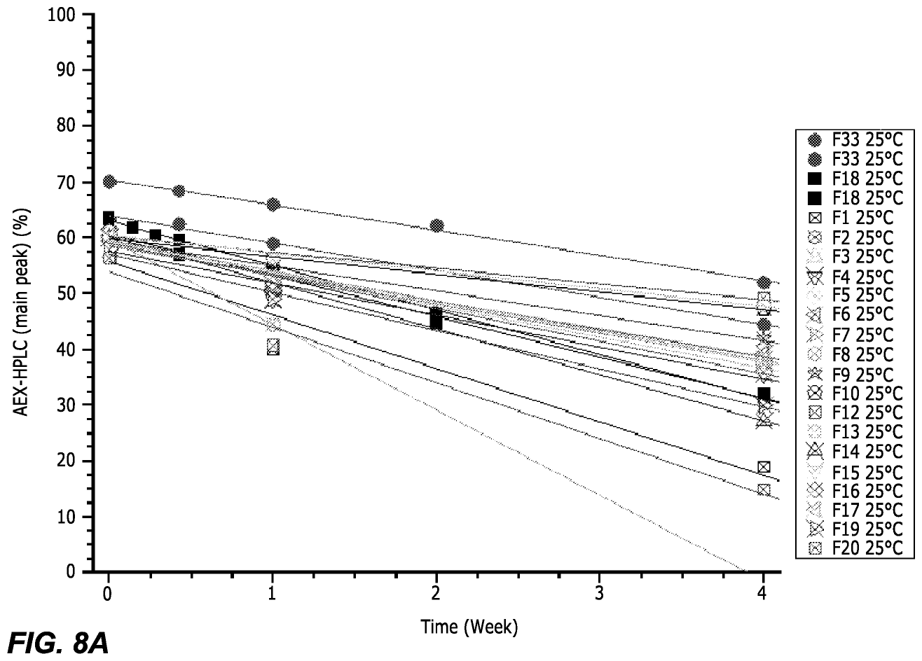

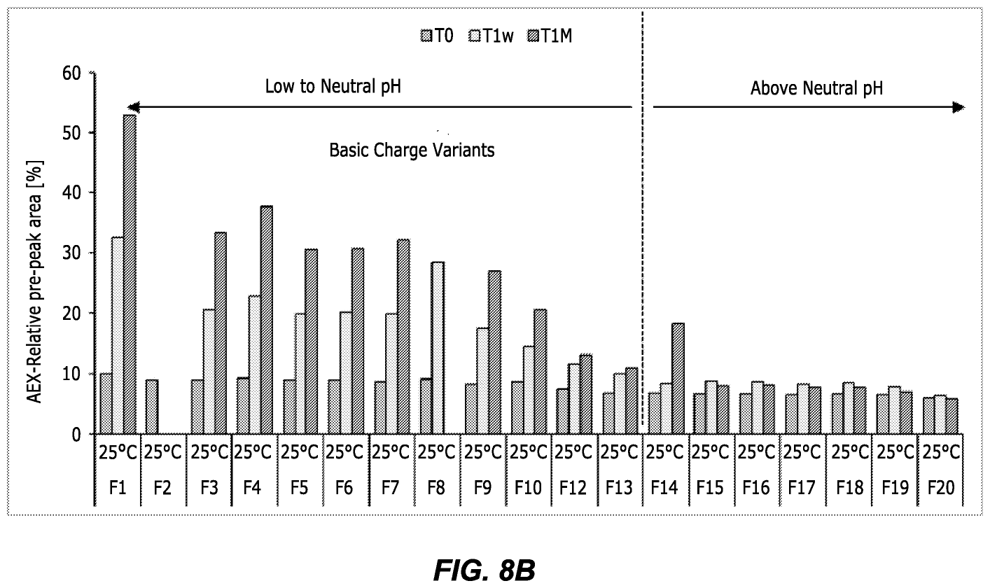

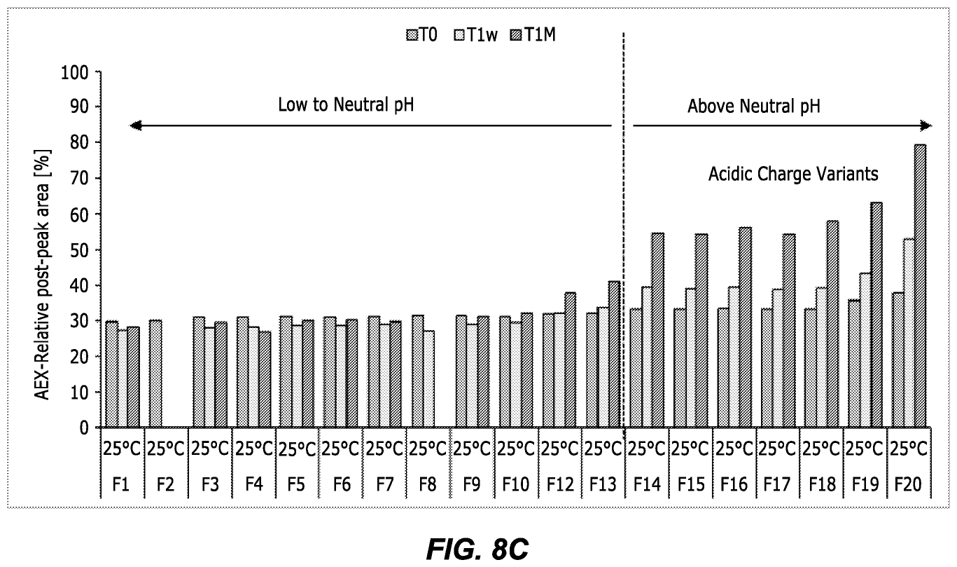

Formation of charge variants when stored at 25° C./60% RH, illustrated as decrease in main peak of EFX over time for F1-F20 by comparison with F33, is shown in FIG. 8A. Notably during 4 weeks storage, EFX formulated in Tris-HCl containing Arg/Arg-HCl, sucrose, PS20 or PS80 (F33 to F38) buffered in the pH range 7.0 to 7.6 demonstrated an approximately 60% slower rate of main peak loss than for EFX in F18 and more than 2-fold slower rate of formation of more acidic charge variants than for EFX in F20.

In addition, FIG. 8B shows relative abundance as percent of total peak area of chromatogram for more basic charge variants (pre-peaks) measured by AEX-HPLC, and FIG. 8C illustrates percent post-peaks, corresponding to more acidic variants at time zero, 1 week, and 1 month at 25° C./60% RH for formulations F1 to F20. As evident from FIG. 8B, basic charge variants or pre-peaks are significantly more abundant in formulations F1 to F12, where F1 shows 53% basic variants, compared to formulations F15 to F20 and F33 where the basic variants remain relatively constant under 10% of the total chromatogram area. In contrast, formulations F15 to F20 manifested greater formation of acidic variants following one month storage at 25° C./60% RH (Table 8).

The improved stability of F33 was also evident when stored at 2-8° C., with the rate of formation of charge variants of EFX being approximately 50% lower than in F18, and 3.2-fold lower than in F20 (Table 9).

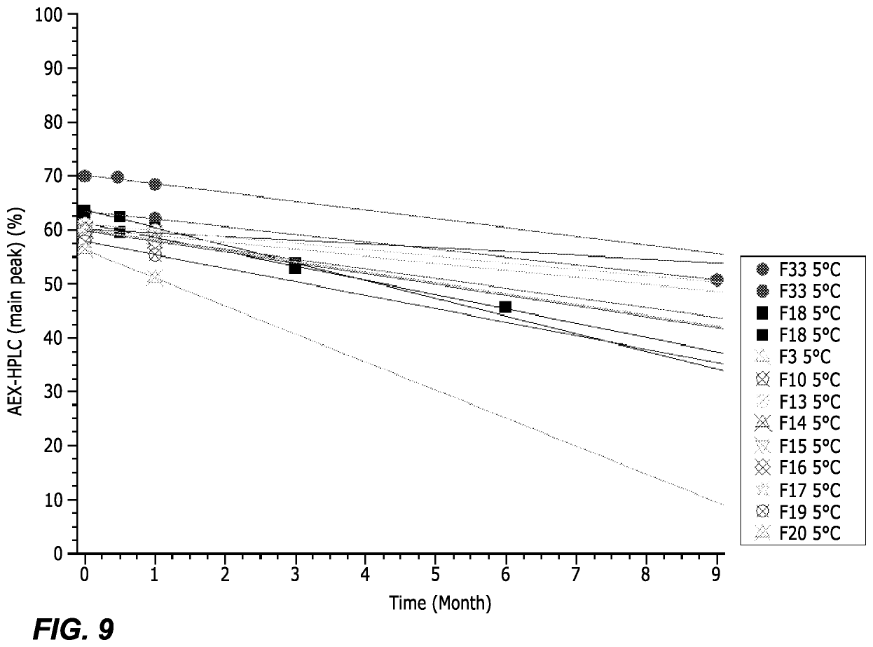

In summary, F33, with a unique combination of excipients (i.e., a sugar, a surfactant, and Arg/Arg-HCl), was the most stable pharmaceutical composition as a liquid formulation (compared to the formulations tested), with the main peak of EFX remaining relatively unchanged over time when stored at 2-8° C. (FIG. 9), as confirmed by the slowest rate of formation of charge variants at 25° C./60% RH by comparison with formulations containing other excipients commonly used in protein-based biopharmaceuticals.

TABLE 8
Rates of formation of EFX charge variants in selected formulations* stored at 25° C.,
expressed as percent purity loss by AEX-HPLC (based on % decrease of main peak area) per week.

| | | Rate of formation |
|---|---|---|
| | | of charge variants |
| Formulation | Composition | [%/week] |
| F33 | 20 mM Tris-HCl, 120 mM Arg/Arg-HCl, 120 mM sucrose 0.06% w/v | −4.68 |
| | PS20, pH 7.3 | |
| F18 | 20 mM Tris/Tris-HCl, 160 mM Lys-HCl, PS20, pH 7.8 | −7.66 |
| F3 | 20 mM Na glutamate/glutamic acid, 220 mM trehalose, PS20, pH 5.0 | −5.55 |
| F14 | 20 mM Na phosphate, 190 mM sucrose, 40 mM Met, PS20, pH 7.5 | −8.25 |
| F15 | 20 mM Na phosphate, 190 mM trehalose, 40 mM Lys-HCl, PS20, pH | −5.38 |
| | 7.5 | |
| F16 | 20 mM Na phosphate, 180 mM sucrose, 40 mM Glu, 40 mM Arg, PS20, | −5.85 |
| | pH 7.5 | |
| F17 | 180 mM sucrose, 40 mM Arg/Arg-HCl, PS20, pH 7.8 | −5.36 |
| F19 | 20 mM Tris/Tris-HCl, 220 mM sucrose, PS20, pH 8.3 | −6.85 |
| F20 | 20 mM Tris/Tris-HCl, 220 mM sucrose, PS20, pH 9.0 | −9.97 |

*EFX Formulations forming gels or phase separating were excluded.

TABLE 9
Rates of formation of EFX charge variants in selected formulations* stored at 2-8°
C., expressed as percent purity loss by AEX-HPLC (as % decrease of main peak area) per month.

| | | Rate of formation |
|---|---|---|
| | | of charge variants |
| Formulation | Composition | [%/month] |
| F33 | 20 mM Tris-HCl, 120 mM Arg/Arg-HCl, 120 mM sucrose 0.06% w/v | −1.52 |
| | PS20, pH 7.3 | |
| F18 | 20 mM Tris/Tris-HCl, 160 mM Lys-HCl, PS20, pH 7.8 | −3.28 |
| F3 | 20 mM Na glutamate/glutamic acid, 220 mM trehalose, PS20, pH 5.0 | −1.70 |
| F14 | 20 mM Na phosphate, 190 mM sucrose, 40 mM Met, PS20, pH 7.5 | −1.80 |
| F15 | 20 mM Na phosphate, 190 mM trehalose, 40 mM Lys-HCl, PS20, pH 7.5 | −2.00 |
| F16 | 20 mM Na phosphate, 180 mM sucrose, 40 mM Glu, 40 mM Arg, PS20, | −2.00 |
| | pH 7.5 | |
| F17 | 180 mM sucrose, 40 mM Arg/Arg-HCl, PS20, pH 7.8 | −2.00 |
| F19 | 20 mM Tris/Tris-HCl, 220 mM sucrose, PS20, pH 8.3 | −2.50 |
| F20 | 20 M Tris/Tris-HCl, 220 mM sucrose, PS20, pH 9.0 | −5.20 |

*EFX Formulations forming gels or phase separating were excluded.

### Evaluation of Size Variants (Aggregation and Fragmentation) by SE-HPLC

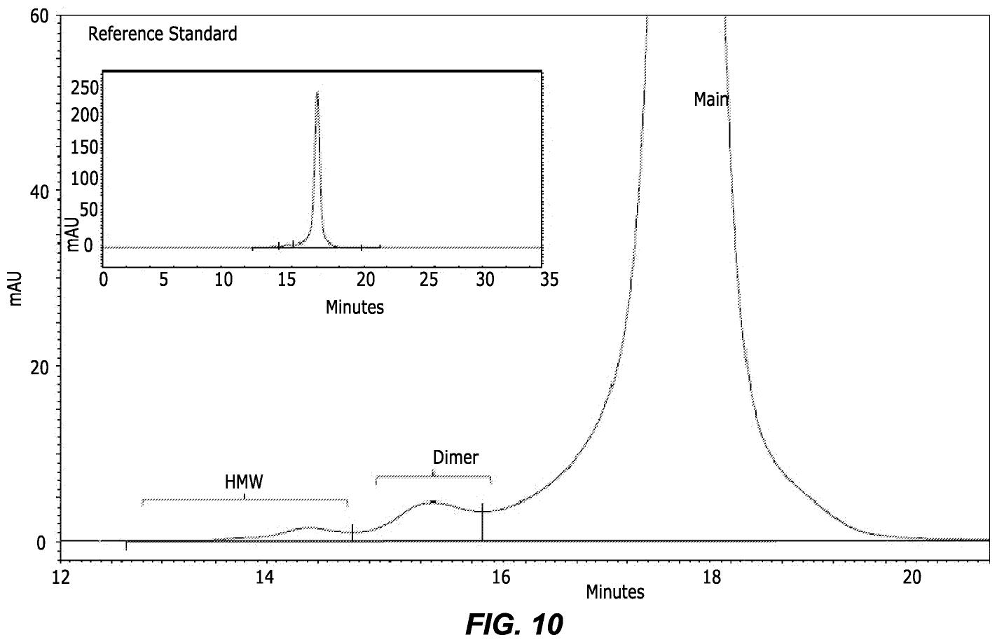

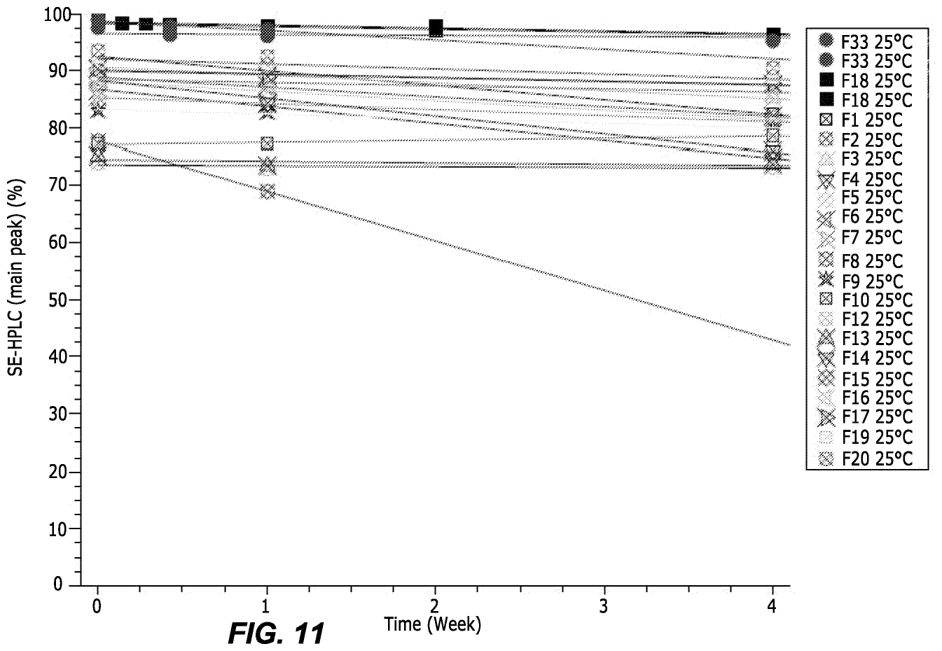

EFX molecular size variants, arising from aggregation (High Molecular Weight Species (HMWS) formation) or fragmentation (Low Molecular Weight Species (LMWS) formation), were characterized by size exclusion high-performance liquid chromatography (SE-HPLC) using a silica gel filtration column (Tosho G3000 SWXL) with UV absorbance detection at 280 nm. The mobile phase was composed of 100 mM sodium phosphate, 500 mM sodium chloride pH 6.9. Size variants of EFX were eluted isocratically from the column at 0.5 mL/min at room temperature with peaks quantitated using a UV absorbance detector. Size variants of EFX are separated into a main species, the predominant chromatographic peak, and low levels of dimer (comprising two EFX homodimers) and high molecular weight (HMW) EFX size variants as shown in FIG. 10.

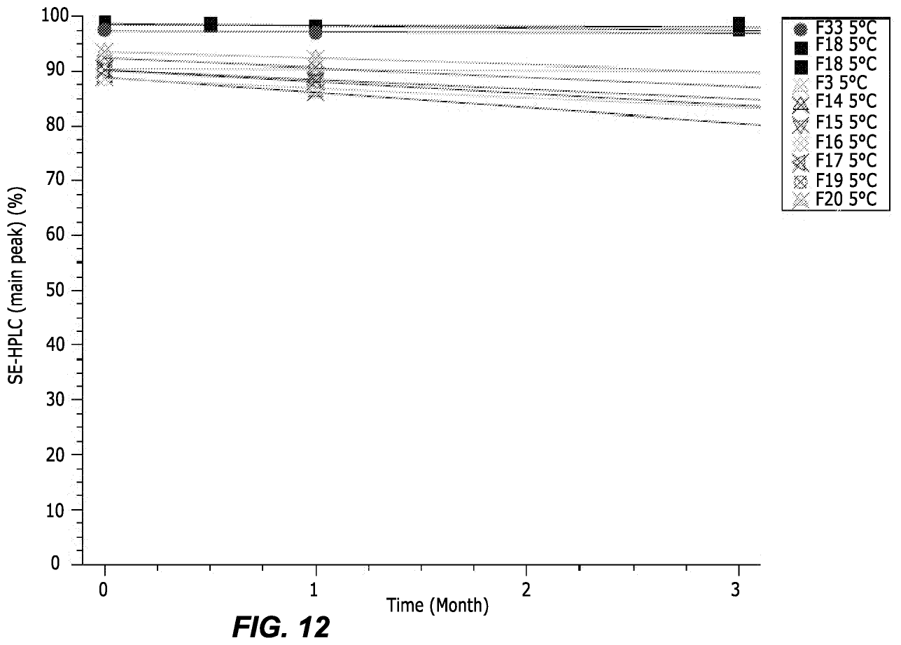

Formation of size variants during storage of selected formulations, F1-F20 and F33 at 25° C., manifested as decreasing main peak of EFX by SE-HPLC, is shown in FIG. 11. Formulations containing Tris-HCl buffer, Arg/Arg-HCl, sucrose, PS20 or PS80 (F33 to F38) buffered at pH range 7.0 to 7.6 had a slower rate of main peak loss than other formulations at 25° C., notably 70% slower than F18, and more than 19.5-fold slower than F14 in contrast (Table 10). When stored at 2-8° C., the rate of formation of size variants (HMWS and LMWS) of EFX in F33 was also approximately 50% lower than for F18, and 20-fold lower than for F14 as examples (Table 11). The unique combination of EFX with the excipients contained in F33 (and at the recited concentrations) ensured that the size variant profile of EFX remained relatively unchanged over time when stored at 2-8° C. (FIG. 12). Likewise, when stored under more stressful conditions at 25° C., F33 demonstrated the slowest rate of formation of size variants (FIG. 11) by comparison with formulations based on other excipients commonly used for protein-based biopharmaceuticals.

TABLE 10
Rates of formation of size variants of EFX in selected formulations* stored at 25°
C., expressed as purity loss (as % decrease of main peak area) by SE-HPLC per week

| | | Rate of formation |
|---|---|---|
| | | of size variants |
| Formulation | Formulation composition | [%/week] |
| F33 | 20 mM Tris-HCl, 120 mM Arg/Arg-HCl, 120 mM sucrose 0.06% w/v | −0.16 |
| | PS20, pH 7.3 | |
| F18 | 20 mM Tris/Tris-HCl, 160 mM Lys-HCl, PS20, pH 7.8 | −0.50 |
| F3 | 20 mM Na glutamate/glutamic acid, 220 mM trehalose, PS20, pH 5.0 | −1.38 |
| F14 | 20 mM Na phosphate, 190 mM sucrose, 40 mM Met, PS20, pH 7.5 | −3.12 |
| F15 | 20 mM Na phosphate, 190 mM trehalose, 40 mM Lys-HCl, PS20, pH | −0.63 |
| | 7.5 | |
| F16 | 20 mM Na phosphate, 180 mM sucrose, 40 mM Glu, 40 mM Arg, PS20, | −0.57 |
| | pH 7.5 | |
| F17 | 180 mM sucrose, 40 mM Arg/Arg-HCl, PS20, pH 7.8 | −0.62 |
| F19 | 20 mM Tris/Tris-HCl, 220 mM sucrose, PS20, pH 8.3 | −0.89 |
| F20 | 20 mM Tris/Tris-HCl, 220 mM sucrose, PS20, pH 9.0 | −0.73 |

*EFX Formulations forming gels or phase separating are excluded.

TABLE 11
Rates of formation of size variants of EFX in selected formulations stored at 2-8°
C. and expressed as percent purity loss (as % decrease of main peak area) by SE-HPLC per month

| | | Rate of formation |
|---|---|---|
| | | of size variants |
| Formulation | Formulation composition | [%/month] |
| F33 | 20 mM Tris-HCl, 120 mM Arg/Arg-HCl, 120 mM sucrose 0.06% w/v | −0.14 |
| | PS20, pH 7.3 | |
| F18 | 20 mM Tris/Tris-HCl, 160 mM Lys-HCl, PS20, pH 7.8 | −0.30 |
| F3 | 20 mM Na glutamate/glutamic acid, 220 mM trehalose, PS20, pH 5.0 | −0.20 |
| F14 | 20 mM Na phosphate, 190 mM sucrose, 40 mM Met, PS20, pH 7.5 | −2.80 |
| F15 | 20 mM Na phosphate, 190 mM trehalose, 40 mM Lys-HCl, PS20, pH | −1.80 |
| | 7.5 | |
| F16 | 20 mM Na phosphate, 180 mM sucrose, 40 mM Glu, 40 mM Arg, PS20, | −1.80 |
| | pH 7.5 | |
| F17 | 180 mM sucrose, 40 mM Arg/Arg-HCl, PS20, pH 7.8 | −2.20 |
| F19 | 20 mM Tris/Tris-HCl, 220 mM sucrose, PS20, pH 8.3 | −1.70 |
| F20 | 20 mM Tris/Tris-HCl, 220 mM sucrose, PS20, pH 9.0 | −1.20 |

*EFX Formulations forming gels or phase separating are excluded.

### Evaluation of Size Variants (HMWS and LMWS) by CE-SDS (Non-Reduced)

Capillary electrophoresis with sodium dodecyl sulfate (CE-SDS) was used to assess the purity of EFX. The method employed reduced, denatured EFX as well as non-reduced, denatured EFX.

Size variants of EFX were quantitatively determined by CE-SDS under denaturing, non-reducing conditions. EFX was mixed with 100 mM sodium phosphate buffer at pH 6.5 and 10% (v/v) SDS solution prior to addition of 140 mM N-ethylmaleimide (NEM) at room temperature. The sample was then analyzed in a 20 cm uncoated silica capillary (50 μm internal diameter) with a Beckman PA800 Plus Pharmaceutical Analysis System attached with a PDA detector monitoring 220 nm absorbance. Data from each electrophoretic analysis was acquired with 32 Karat data acquisition software.

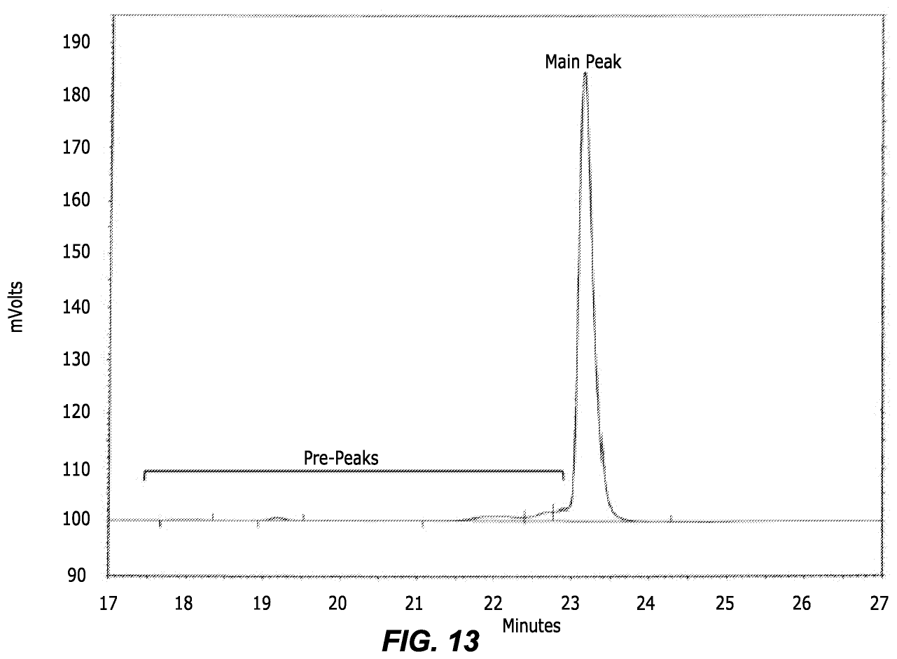

Analysis of the non-reduced, denatured EFX showed intact protein as main peak. Single chain and low molecular weight species migrate before the main peak as pre-peaks. Aggregates of EFX were integrated after the main peak as post-peaks, as shown in a representative electropherogram for EFX (FIG. 13).

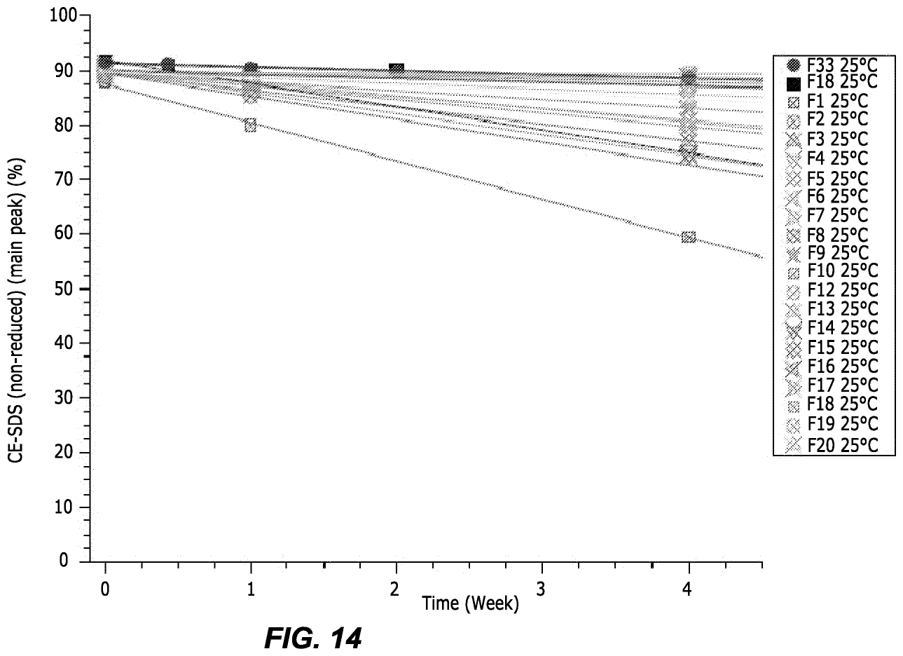

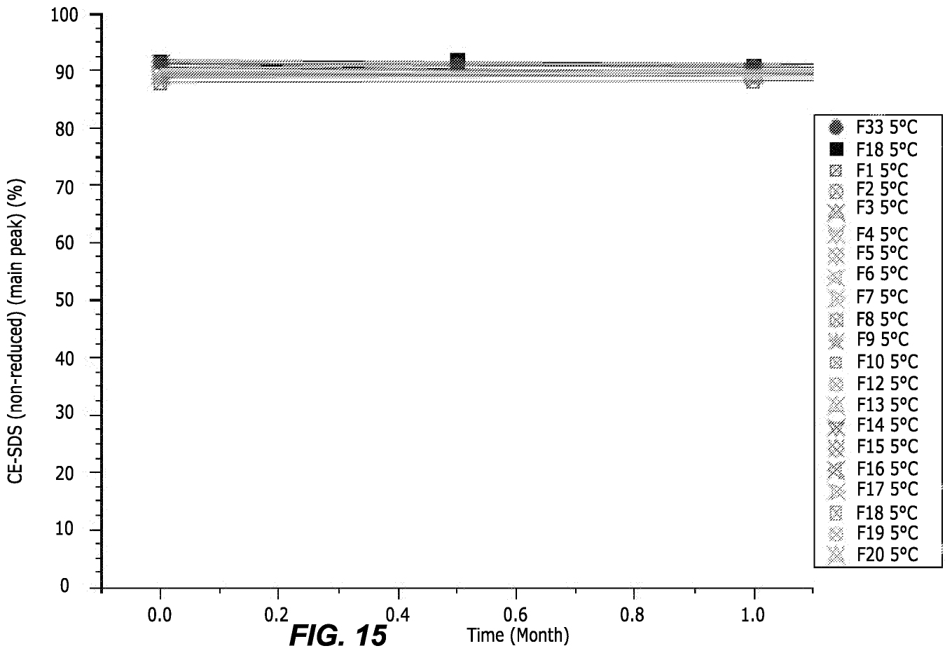

Formation of size variants of EFX in selected formulations were stored at 25° C., evident as decreasing main peak over time by CE-SDS (non-reduced), shown in FIG. 14, and when stored at 2-8° C. in FIG. 15. Size variants in all formulations remained relatively unchanged over time when EFX formulations were stored at 2-8° C. (FIG. 15). At 25° C., purity loss was greater, for example in F1 (−7.04%/week). By comparison, F33 demonstrated improved stability under these conditions with size variants formed at an approximately 10-fold slower rate (−0.70%/week).

### Evaluation of Formation of Size Variants (HMWS and LMWS) by RP-HPLC

The RP-HPLC method separates EFX on a Zorbax 300SB C18 (4.6 mm×150 mm, 3.5 μm particle size) column using a mobile phase of 0.1% (v/v) trifluoroacetic acid in water over a biphasic gradient of 70% N-propanol and 30% acetonitrile at 45° C., and a flow rate of 0.5 mL/min. Eluted protein peaks are detected with a UV absorbance detector at 280 nm.

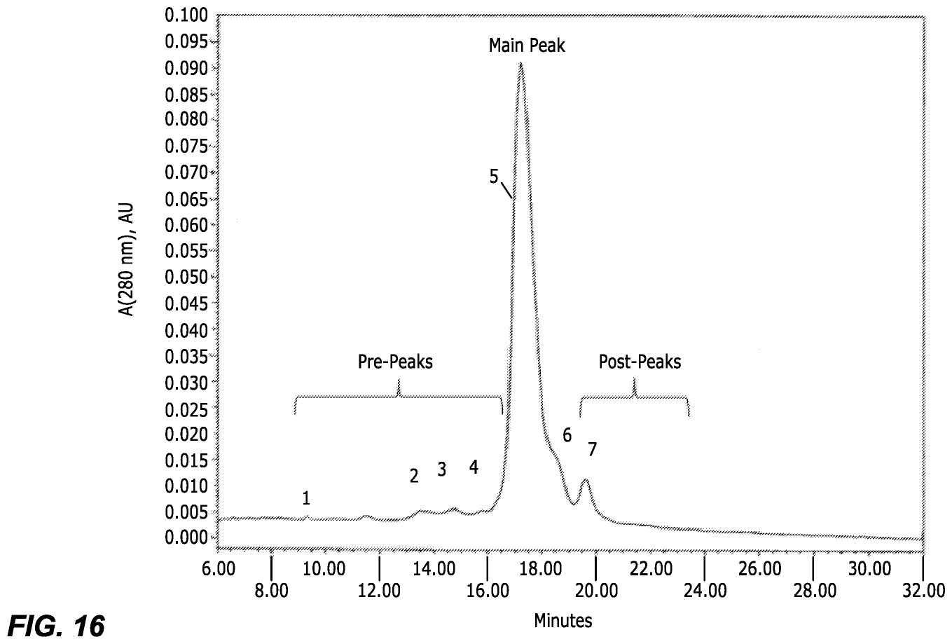

The RP-HPLC chromatogram of EFX is shown in FIG. 16. The content of separated peaks (numbered 1 to 7) was characterized by online high-resolution mass spectrometry and summarized in Table 12.

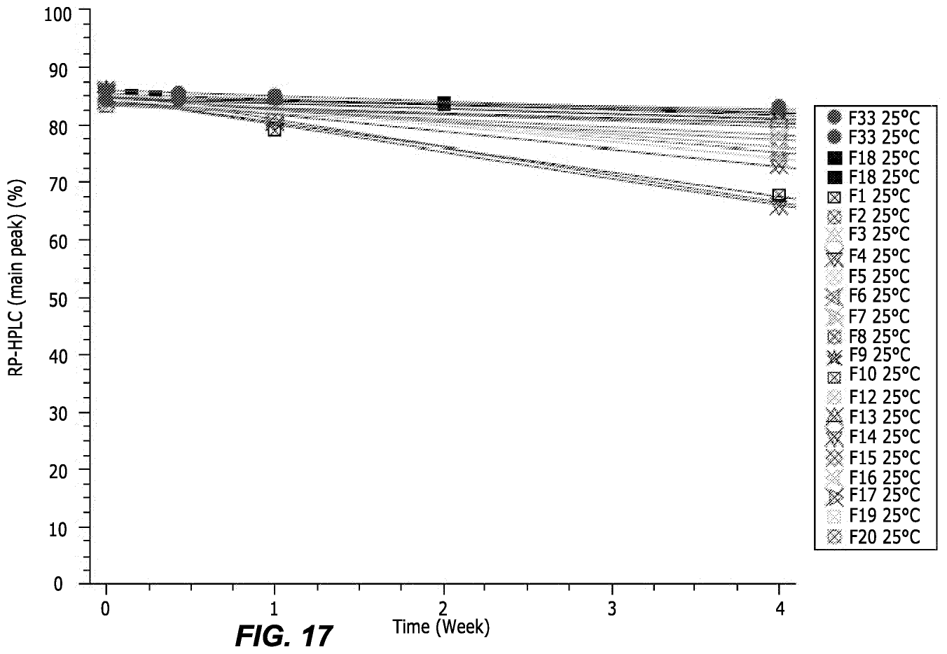

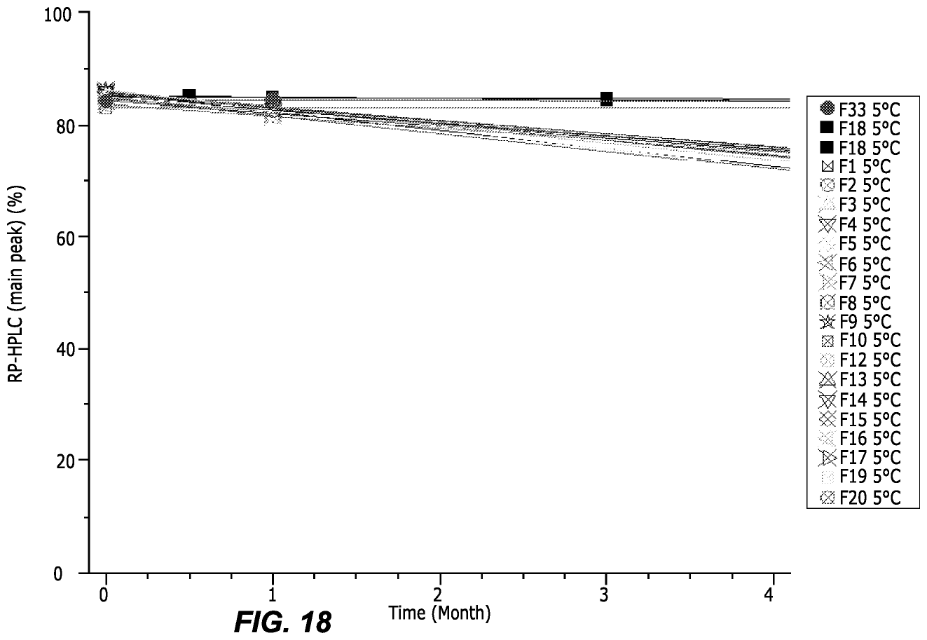

Formation of size variants in selected formulations F1-F20 and F33 stored at 25° C., evident as decreasing main peak over time measured by RP-HPLC, is shown in FIG. 17. Formulations of EFX containing Tris-HCl buffer, Arg/Arg-HCl, sucrose, PS20 or PS80 (F33 to F38) buffered at pH range 7.0 to 7.6 have approximately 10% slower rate of main peak loss than formulation F18 at 25° C., and approximately 6-fold slower rate than formulation F14 (Table 10). When stored at 2-8° C., the rate of formation of size variants (HMWS and LMWS) in F33 was also approximately half that of F18, and approximately 30-fold lower than the rate for F20 (Table 13).

While various formulations tested provided beneficial effects on stability of EFX, the combination of EFX with the excipients contained in F33 (i.e., a sugar, a surfactant, and Arg/Arg-HCl) was superior, ensuring that the size variant profile of EFX remained relatively unchanged over time when stored at 2-8° C. (FIG. 18). Likewise, when stored under more stressful conditions at 25° C., F33 demonstrated the slowest rate of formation of size variants (FIG. 17) by comparison with formulations based on other excipient combinations commonly used for protein-based biopharmaceuticals.

TABLE 12
Rates of formation of size variants in selected formulations of EFX stored at 25°
C., expressed as purity loss (as % decrease of main peak area) by RP-HPLC per week.

| | | Rate of formation |
|---|---|---|
| | | of size variants |
| Formulation* | Formulation composition | [%/week] |
| F33 | 20 mM Tris-HCl, 120 mM Arg/Arg-HCl, 120 mM sucrose 0.06% w/v | −0.75 |
| | PS20, pH 7.3 | |
| F18 | 20 mM Tris/Tris-HCl, 160 mM Lys-HCl, PS20, pH 7.8 | −0.83 |
| F3 | 20 mM Na glutamate/glutamic acid, 220 mM trehalose, PS20, pH 5.0 | −2.77 |
| F14 | 20 mM Na phosphate, 190 mM sucrose, 40 mM Met, PS20, pH 7.5 | −4.66 |
| F15 | 20 mM Na phosphate, 190 mM trehalose, 40 mM Lys-HCl, PS20, pH | −1.41 |
| | 7.5 | |
| F16 | 20 mM Na phosphate, 180 mM sucrose, 40 mM Glu, 40 mM Arg, | −1.53 |
| | PS20, pH 7.5 | |
| F17 | 180 mM sucrose, 40 mM Arg/Arg-HCl, PS20, pH 7.8 | −0.87 |
| F19 | 20 mM Tris/Tris-HCl, 220 mM sucrose, PS20, pH 8.3 | −1.09 |
| F20 | 20 mM Tris/Tris-HCl, 220 mM sucrose, PS20, pH 9.0 | −2.18 |

*EFX Formulations forming gels or phase separating are excluded.

TABLE 13
Rates of formation of size variants in selected formulations of EFX stored at 2-8°
C., expressed as purity loss (as % decrease of main peak area) by RP-HPLC per week.

| | | Rate of size |
|---|---|---|
| | | variants formation |
| Formulation* | Formulation composition | [%/month] |
| F33 | 20 mM Tris-HCl, 120 mM Arg/Arg-HCl, 120 mM sucrose 0.06% w/v | −0.08 |
| | PS20, pH 7.3 | |
| F18 | 20 mM Tris/Tris-HCl, 160 mM Lys-HCl, PS20, pH 7.8 | −0.23 |
| F3 | 20 mM Na glutamate/glutamic acid, 220 mM trehalose, PS20, pH 5.0 | −2.30 |
| F14 | 20 mM Na phosphate, 190 mM sucrose, 40 mM Met, PS20, pH 7.5 | −2.20 |
| F15 | 20 mM Na phosphate, 190 mM trehalose, 40 mM Lys-HCl, PS20, pH | −2.20 |
| | 7.5 | |
| F16 | 20 mM Na phosphate, 180 mM sucrose, 40 mM Glu, 40 mM Arg, | −2.10 |
| | PS20, pH 7.5 | |
| F17 | 180 mM sucrose, 40 mM Arg/Arg-HCl, PS20, pH 7.8 | −2.30 |
| F19 | 20 mM Tris/Tris-HCl, 220 mM sucrose, PS20, pH 8.3 | −3.20 |
| F20 | 20 mM Tris/Tris-HCl, 220 mM sucrose, PS20, pH 9.0 | −2.50 |

*EFX Formulations forming gels or phase separating were excluded from RP-HPLC analysis.

### Evaluation of Mass and Charge Variants of EFX in Most Stable Formulations by Mass Spectrometry

To elucidate mass variants and posttranslational modifications associated with altered charge variants, various formulations of EFX were characterized by intact mass and by peptide mapping after digestion with trypsin. To minimize chemical modifications such as deamidation or oxidation arising during preparation of tryptic peptides, digestion was performed under reducing conditions. The resulting tryptic peptides were separated using a C18 reverse phase ultra-performance liquid chromatography (RP-UPLC) with detection by UV absorption (280 nm), then characterized by high resolution mass spectrophotometry analysis. The types of mass variants of EFX in unstressed formulations (stored frozen) were compared with those in formulations stored for 1 month at 2-8° C., 1 month at 25° C. and 1 month at 40° C. (data not shown due to similarity with 1 month at 25° C.). The relative abundance of unmodified intact homodimer of EFX, and of other mass variants (fragments and modified species) in the most stable formulations, F18 and F33, is summarized in Table 14.

TABLE 14
Distribution of Mass Variants of EFX measured by LC/MS in formulations F18 and F33 stored
for 1 month at 25° C., by reference to the same formulations under unstressed conditions.

| | Theoretical | Percent Abundance by MS of EFX Mass Variants | | | | |
|---|---|---|---|---|---|---|
| EFX Mass | Intact | EFX F18 | EFX F18 | EFX F33 | EFX F33 | EFX F33 |
| Variants | Mass (Da) | Unstressed | 1 M 25° C. | Unstressed | 1 M 2-8° C. | 1 M 25° C. |
| EFX | 92,108.8 | 89.3 | 78.7 | 88.6 | 89.3 | 87.6 |
| (homodimer | | | | | | |
| unmodified) | | | | | | |
| EFX, 6-424 | 91,496.10 | 1.7 | 1.0 | 1.9 | 1.0 | 1.8 |
| EFX, 1-412 | 90,915.70 | 5.1 | ND | ND | ND | ND |
| EFX, 1-411 | 90,816.50 | 0.6 | 3.6 | 4.7 | 4.7 | 4.8 |
| EFX, 1-397 | 89,408.00 | 1.1 | ND | ND | ND | ND |
| EFX, 1-396 | 89,336.90 | 0.3 | ND | ND | ND | ND |
| EFX, 1-377 | 87,376.50 | ND | ND | ND | ND | ND |
| EFX, 1-370 | 86,729.80 | ND | 0.4 | 0.8 | 0.9 | 0.9 |
| EFX, 1-364 | 86,009.00 | 0.2 | 1.5 | ND | ND | 0.4 |
| EFX, 1-298 | 78,704.70 | ND | 0.5 | 0.9 | 0.9 | 1.1 |
| EFX, 1-296 | 78,463.40 | 0.1 | ND | 0.1 | 0.2 | 0.1 |
| EFX, 1-278 | 76,679.60 | 0.2 | ND | 0.4 | 0.4 | 0.4 |
| EFX, 1-276 | 76,437.30 | ND | ND | 0.5 | 0.5 | 0.6 |
| EFX, 1-274 | 76,187.00 | 0.04 | ND | 0.5 | 0.5 | 0.6 |
| EFX, 1-266 | 75,328.20 | 0.2 | ND | 0.1 | ND | ND |
| EFX, 1-255 | 74,005.70 | 1.2 | 0.3 | 0.2 | 0.2 | 0.3 |
| EFX (single | 46,054.40 | 0.4 | ND | 0.1 | 0.2 | 0.3 |
| monomer) | | | | | | |
| EFX + 42 Da⁽¹⁾ | NA | 0.8 | ND | 1.0 | 1.1 | 1.0 |
| EFX + 34 Da | NA | ND | ND | ND | ND | ND |
| EFX + 16 Da | NA | ND | 14.0 | ND | ND | ND |

MS = mass spectrometry;
ND = not detected
⁽¹⁾A common impurity of +42 Da corresponds to acetylation of the N-terminus or a basic residue.

The types of posttranslational modification were identified for each tryptic peptide. The relative abundance of each peptide containing a modified amino acid residue from the most stable formulations of EFX (F18 and F33) was compared after stressing for 1 month at 25° C. by reference to the same formulations unstressed, see Table 15.

TABLE 15
LC/MS Peptide Map Analysis of EFX from Unstressed
and Stressed formulations, F18 and F33.

| | Percent of Modified Peptide | | | | | |
|---|---|---|---|---|---|---|
| | | EFX F18 | EFX F18 | EFX F33 | EFX F33 | EFX F33 |
| Peptide | Type | Unstressed | 1 M 25° C. | Unstressed | 1 M 2-8° C. | 1 M 25° C. |
| TA | Oxidation | 0.2 | ND | ND | ND | ND |
| TB | Oxidation | 1.8 | 3.8 | 1.3 | 1.4 | 1.8 |
| TC | Succinimide | 0.3 | 0.2 | 0.4 | 0.8 | 0.5 |
| | Deamidation | 0.8 | 0.7 | ND | 0.1 | ND |
| TD | Deamidation | 0.7 | ND | ND | ND | ND |
| TE | Succinimide | ND | ND | 0.3 | 0.2 | 0.2 |
| | Deamidation | 2.2 | 15.2 | ND | ND | ND |
| TF | Succinimide | 3.4 | 3.2 | 2.6 | 2.7 | 2.5 |
| | Deamidation | 3.9 | 1.9 | 2.0 | 2.5 | 2.0 |
| TG | Succinimide | 0.3 | ND | 0.1 | 0.1 | 0.1 |
| | Deamidation | 0.8 | 0.3 | ND | ND | ND |
| TH | Deamidation | 0.8 | 0.5 | ND | ND | ND |
| | Succinimide | 3.0 | 2.6 | 2.3 | 2.0 | 1.8 |
| TI | Deamidation | 5.7 | 7.9 | 2.0 | 4.0 | 4.5 |
| TJ | Succinimide | ND | 0.3 | ND | ND | ND |
| | Deamidation | 0.5 | 2.1 | 1.2 | 3.0 | 4.6 |
| TK | Deamidation | ND | 0.4 | ND | 0.2 | ND |
| TL | Oxidation | 0.7 | 2.1 | 0.4 | 0.4 | 1.1 |
| TM | Succinimide | 0.3 | 0.5 | 0.4 | 0.1 | 0.5 |
| | Deamidation | 2.0 | 0.4 | 0.3 | 0.1 | 0.5 |
| TN | Deamidation | ND | ND | ND | ND | ND |
| TO | Deamidation | ND | ND | ND | ND | ND |
| TP | Deamidation | 0.0 | 5.3 | ND | ND | ND |
| TQ | Oxidation | 1.9 | 10.1 | 1.2 | 1.5 | 2.0 |

ND = not detected

The main form of posttranslational modification of EFX, formulated in F18 and F33, is deamidation of asparagine (Asn) to acidic aspartic acid residues, including low levels of the succinimide intermediate of Asn, resulting in more acidic charge variants. Eight of nine Asn residues in each monomeric chain of EFX showed some degree of deamidation. In addition, two of four glutamine (Gln) residues were also deamidated to acidic glutamic acid in each monomeric chain. Oxidation of methionine (Met) was present at low levels in three positions. As expected, a significantly lower level of posttranslational modifications and charge variant formation is evident under stress in the most stable formulations of EFX, F18 and F33, with F33 appearing less susceptible than F18, especially the two Asn residues most susceptible to deamidation and a Met susceptible to oxidation (see Table 15).

### Evaluation of Size Variants of EFX in Most Stable Formulations by Size Exclusion Chromatography-Multiple Angle Laser Light Scattering (SEC-MALLS)

The distribution of species by molecular weight in undiluted samples of EFX, was assessed by SEC-MALLS. The SEC-MALLS method monitors elution of different sized species of EFX using two in-line detectors: 1) a UV detector recording at 280 nm and 360 nm, (1260 Infinity LC, Agilent Technologies), and 2) a light scattering detector (DAWN HELEOS II, Wyatt Technology). Data analysis and molecular weight (MW) calculations were performed on MALLS data using ASTRA 6 software (Wyatt Technology). A theoretical extinction coefficient of 0.97 mL mg⁻¹ cm⁻¹ for EFX was used to calculate molecular weight values.

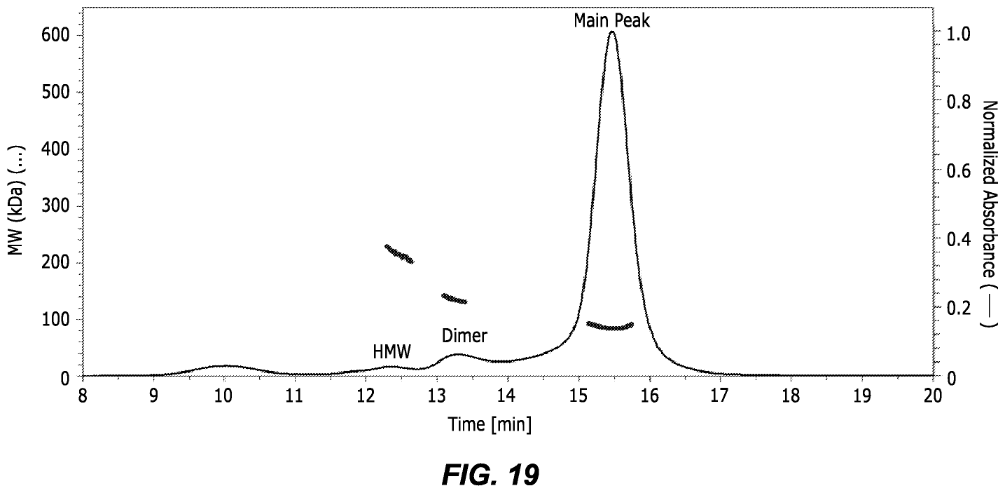

Analysis of replicate injections of formulations of EFX revealed the main peak accounted for 94.5% of protein, assigned an apparent MW of 88.0 to 88.4 kDa, in good agreement with the calculated MW of 92 kDa. Relatively low amounts of three additional species were also found, including two high MW variants: dimer and HMW, and a low MW variant (LMW). FIG. 19 shows a representative SEC-MALLS chromatogram of EFX in F18 (unstressed). Precise determination of the molecular weights of the low abundance variants was challenging because of relatively incomplete separation of the various peaks as a result of the high protein concentration required for SEC-MALLS. Nevertheless, the dimer species had an apparent MW of 136 kDa, and the HMW species an apparent MW of 292 kDa, implying the HMW species may represent a larger oligomer, possibly a tetramer. The LMW species was not sufficiently abundant to assign a MW.

### Size Distribution by Sedimentation Velocity Analytical Ultracentrifugation (SV-AUC) in Most Stable Formulations

The hydrodynamic conformational properties of EFX in different formulations were analyzed by SV-AUC using an Optima analytical ultracentrifuge (Beckman-Coulter). Samples were analyzed at 1.0 mg protein/mL. SV-AUC run was performed at 20° C., with 12-mm Epon-charcoal double sector centerpiece sample cells in an 8-hole An50 titanium rotor at 45,000 rpm. Global fitting of raw sedimentation boundary data, selected from a subset of radial scan measurements, was performed for each sample by the continuous distribution c(s) analysis method using software program SEDFIT V.11.71. In addition to producing sedimentation coefficient distribution c(s) profiles, the sedimentation coefficients under standard conditions (S_{20,w}), the frictional coefficient ratio (f/f₀), and molecular weight (MW) were estimated.

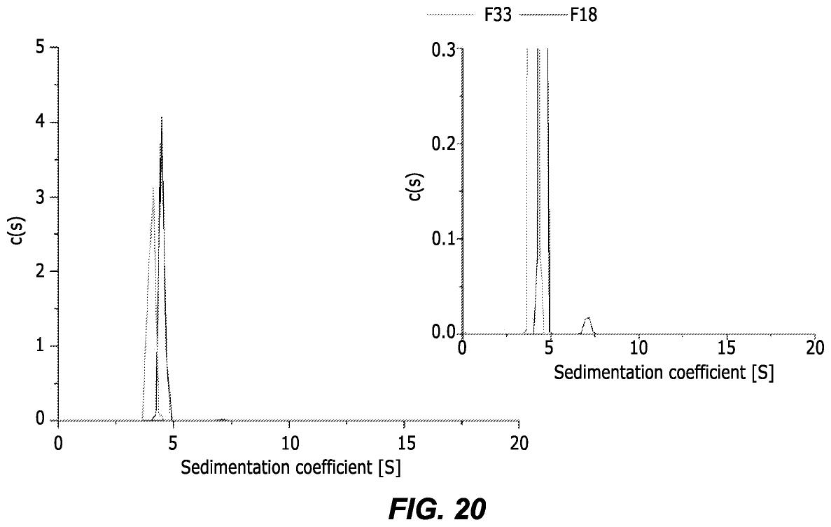

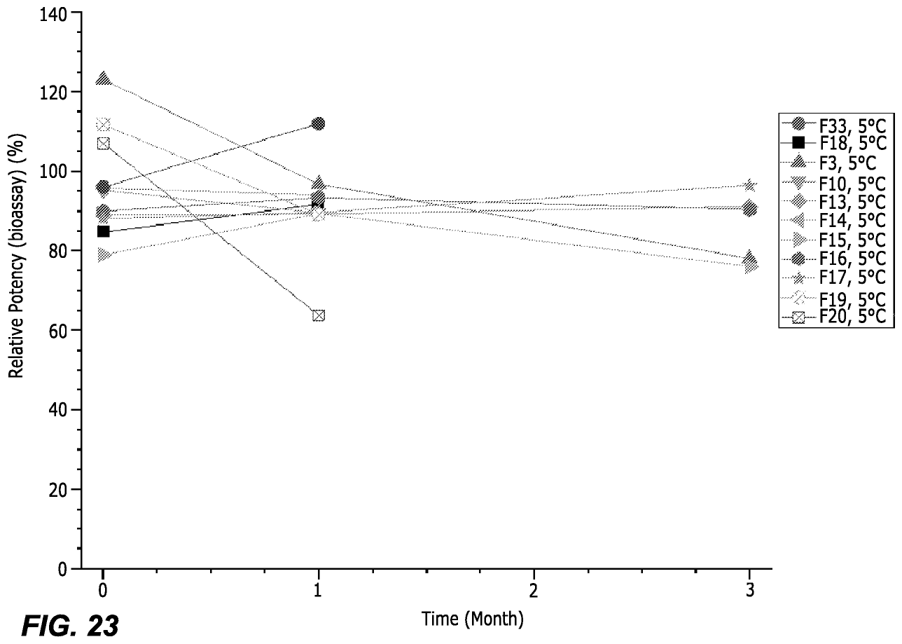

Representative sedimentation coefficient distribution profiles of EFX in F18 and F33 are shown in FIG. 20, where the vertical axis of the graph shows the concentration distribution and the horizontal axis shows the separation of the species on the basis of their sedimentation coefficient. The main species in formulation F33, accounting for approximately 100% of total species, has an apparent sedimentation coefficient of 4.06 S, f/f₀ of 1.8, and a calculated apparent MW 89.7 kDa (Table 16). The main species in formulation F18, accounting for 98.7% of total species, has an apparent sedimentation coefficient of 4.47 S, f/f₀ of 1.6, and a calculated apparent MW 88.9 kDa (Table 16). HMW species account for 0.2% to 1.3% of total species. No LMW species are observed in F18 (FIG. 23 with scale expanded insert). Sedimentation coefficient values for the HMW species are consistent with dimer (˜6.5 S) and a larger oligomeric species, potentially a tetramer (˜9 S).

TABLE 16
Hydrodynamic Parameters for Main Species
of EFX in F18 and F33 by SV-AUC Analysis.

SV-AUC Analysis by SEDFIT
Main Species

| Sample | | s_{20, w} | Apparent MW | Content |
|---|---|---|---|---|
| name | f/f0 | (S) | (kDa) | (%) |
| EFX F18 | 1.6 | 4.47 | 88.9 | 98.7 |
| EFX F33 | 1.8 | 4.06 | 89.7 | 100.0 |

f/f0 = frictional coefficient ratio;
MW = molecular weight;
S_{20, w} = sedimentation coefficient under standard conditions;
SV-AUC = sedimentation velocity analytical ultracentrifugation

### Cell-Based Potency Bioassay

EFX cell-based potency bioassay uses “iLite FGF21 Assay Ready Cells” (Svar Life Sciences, Cat #BM3071), derived from the human embryonic kidney cell line, HEK293. These FGF21 Assay Ready Cells have been recombinantly engineered to overexpress: (1) two obligate co-receptors of human FGF21: human Fibroblast Growth Factor Receptor-1c (FGFR1c) and human βKlotho (KLB), and (2) a reporter system designed to express firefly luciferase in response to downstream intracellular signal transduction from activated FGFR1c (Ogawa et al., Proc. Natl. Acad. Sci. U.S.A 104, 7432-7437; Agrawal et al., Mol Metab. 2018; 13:45-55; Yie et al., FEBS Lett. 583, 19-24). When bound as a co-receptor complex with KLB, FGF21 activates the tyrosine kinase of FGFR1c, which in turn phosphorylates downstream adaptor proteins resulting in activation of the rat sarcoma—mitogen activated protein kinase (RAS-MAPK) cascade, including phosphorylation of ERK1/2 (extracellular signal regulated protein kinase). Phosphorylated ERK1/2 translocates to the nucleus where it activates the transcription factor, ETS domain-containing protein Elk1 (Ornitz and Itoh, Wiley Interdiscip Rev Dev Biol. 2015; 4(3):215-66; Zou et al, Mol Med Rep. 2019 February; 19(2):759-770). Therefore, iLite FGF21 Assay Ready Cells enable the in vitro potency of EFX as an agonist of FGF21's co-receptor complex of FGFR1c-KLB to be measured by a cell-based assay. The trimeric complex of EFX simultaneously binding to the co-receptors stimulates expression of luciferase enzyme in proportion to the extent EFX activates FGFR1c mediated signal transduction.

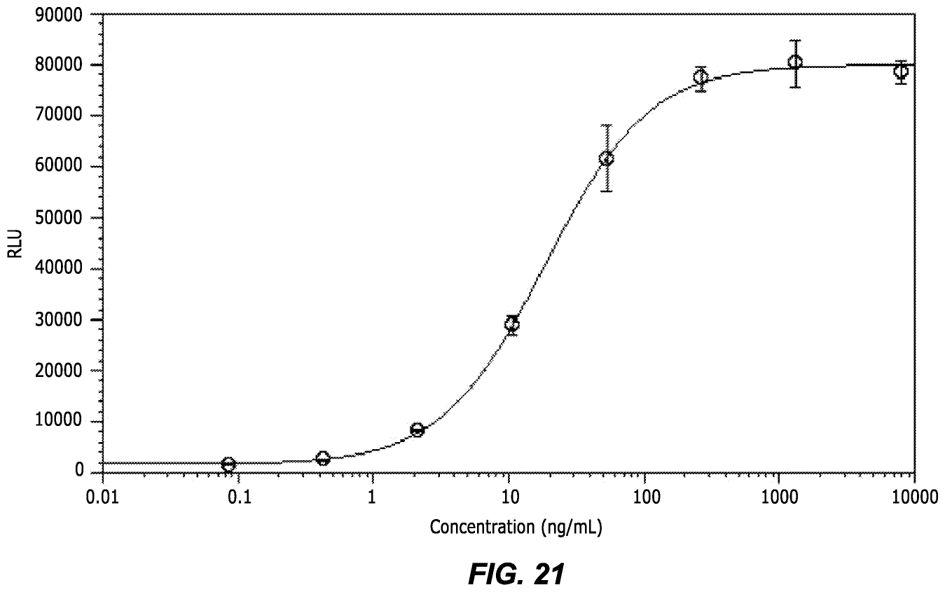

The iLite FGF21 Assay Ready cells are plated, then incubated with serial dilutions of EFX test samples and appropriate positive and negative controls run in parallel. To measure the quantity of luciferase expressed, cells are lysed with reagent containing detergent and the luciferase substrate, luciferin. Cleavage of luciferin by luciferase produces luminescence, measured using a luminometer. The luminescence signal is plotted as a function of test article protein concentration, producing a concentration-response curve that is fit to a four-parameter logistic equation by non-linear least squares regression analysis. Relative potency of test samples is determined by constraining the lower/upper asymptotes and Hill slope to the values of a curve fit to a concurrently generated Reference Standard concentration-response plot, then taking the ratio of EC50 of the Reference Standard to the EC50 parameter of the sample. A representative concentration-response curve for EFX in F18 is shown in FIG. 21.

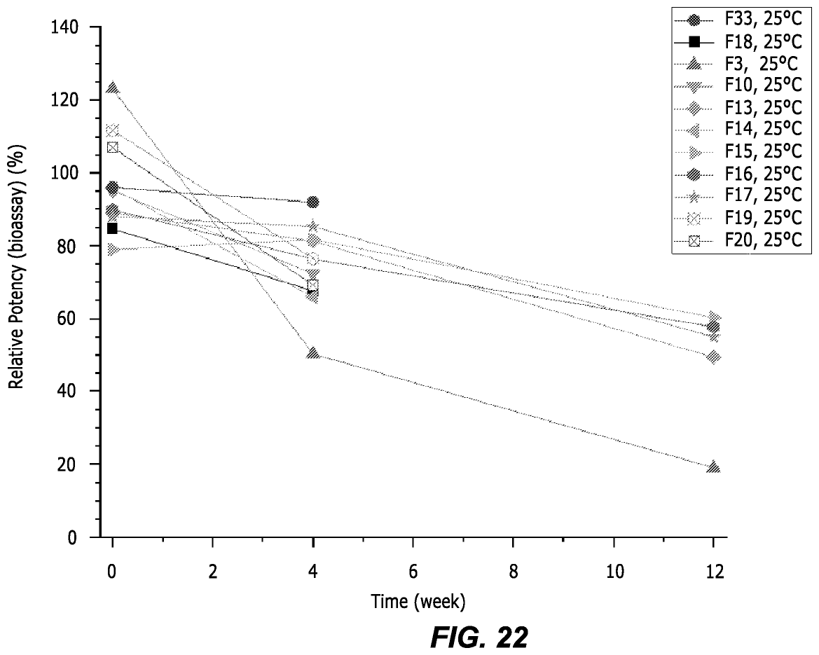

The relative potency of EFX in selected formulations F1-F20 and F33, stored at 25° C., measured by the i-Lite cell-based bioassay, is shown in FIG. 22. Relative potency of EFX in selected formulations stored at 2-8° C. is shown in FIG. 23. Under both storage conditions, the formulations of EFX containing Tris-HCl buffer, Arg/Arg-HCl, sucrose, PS20 or PS80 (F33 to F38) buffered at pH range 7.0 to 7.6 show no apparent loss of relative potency over time, in contrast to many of the other formulations such as F3 which showed approximately 80% loss of potency over 12 weeks storage at 25° C./60% RH.

The unique combination of EFX with the excipients contained in F33 ensured that the cell-based potency of EFX remained relatively unchanged over time when stored at 25° C. in contrast to formulations based on other excipients commonly used for protein-based biopharmaceuticals (FIG. 22).

### Example 4: Conformational and Thermal Stability of EFX Formulations

The Fourier Transformed Infrared (FTIR) spectra of solutions containing EFX protein were obtained on a Tensor 27 FTIR spectrometer (Bruker Optics) and an AquaSpec transmission optical bench at a controlled temperature of 25° C. The spectra of protein samples measured without dilution, were recorded at wavenumbers from 4,000 to 850 cm⁻¹ with a resolution of 4 cm⁻¹. Each single-beam measurement was an average of 60 scans and used atmospheric compensation (elimination of interfering H₂O and/or CO₂ bands in the spectra). The background-corrected absorbance spectrum of each sample containing EFX was transformed into a second derivative spectrum after vector normalization in the wavenumber region of 1,700 to 1,600 cm⁻¹ and with 9 smoothing points.

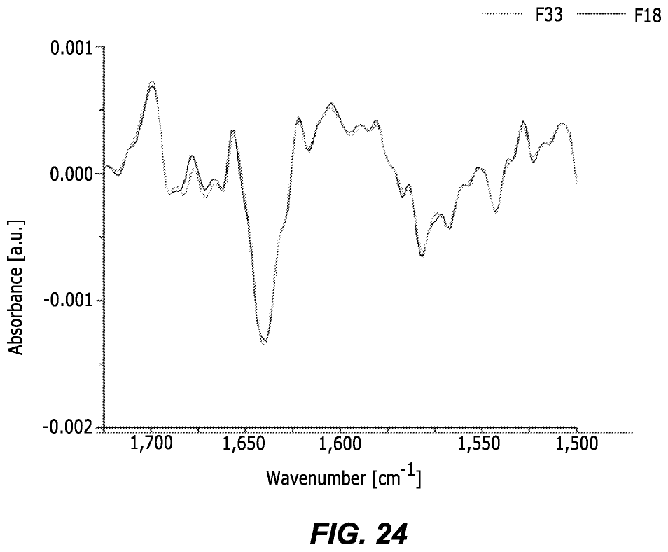

The second derivative FTIR spectrum of EFX in F18 and F33 are shown in FIG. 24. The absorption spectrum exhibits strong bands at around 1641 cm⁻¹ and 1689 cm⁻¹ corresponding to beta-sheets, indicating the predominance of anti-parallel beta-sheet structure in the protein, typical of proteins containing an Fc domain.

### Conformational Stability by Far-UV Circular Dichroism Spectroscopy (CD)

The secondary structure of EFX was analyzed by far-UV Circular Dichroism Spectroscopy (CD). The primary chromophore for far-UV CD spectroscopy (190-260 nm) is a protein's peptide bonds. The CD signal, as a function of wavelength, arises from the orientation of peptide bonds underlying the secondary structure, which is determined by a protein's sequence. A far-UV CD spectrum, therefore, provides a sensitive measurement of a protein's secondary structure.

A Chirascan Auto Q100 CD spectrometer (Applied Photophysics Ltd.) was used for automated Far-UV CD spectroscopy measurements (wavelength range: 190-260 nm). Spectra were collected at 20° C. with a protein concentration of 2.0 mg/mL and a pathlength of 0.1 mm. The spectral bandwidth was set to 1.0 nm, the sampling time per point was 1.0 sec, and the step size was 1.0 nm. Ten consecutive scans were averaged for each measurement of a protein sample. A reference spectrum was recorded for the formulation buffer prior to measuring each protein sample, then subtracted from the protein's spectrum. After subtraction, CD values were converted to mean residue ellipticity ([Θ]mr) values.

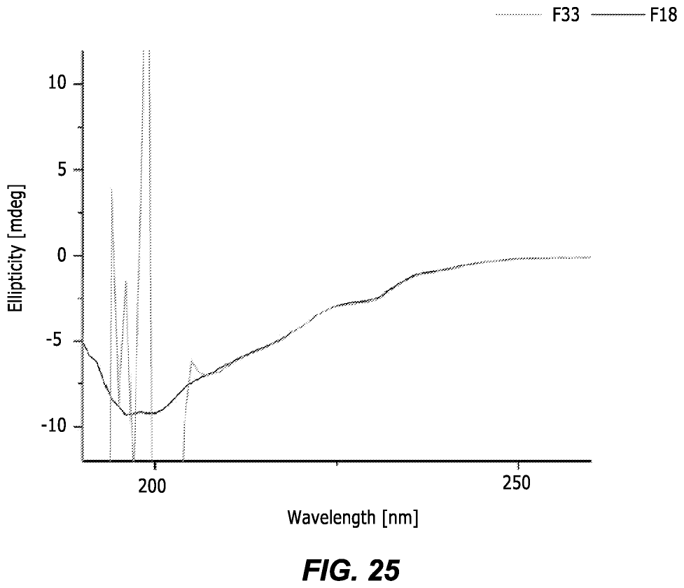

The far-UV CD spectra of EFX in formulations F18 and F33 (195-260 nm) (FIG. 25) are consistent with that of other proteins incorporating an Fc-domain (Li et al, 2012). The spectrum has a small shoulder feature at 230 nm indicating a properly folded Fc domain. Primary negative ellipticity of the CD spectrum is typical of proteins in the Fibroblast Growth Factor super family, which includes FGF21, indicating the FGF21 polypeptide chains of EFX are properly folded (Xu et al, 2012). However, it is to be noted that the presence of some excipients such as L-Lysine and polysorbate 20 in some formulations containing EFX substantially increases background absorbance in the far-UV wavelength region of the spectrum, particularly at wavelengths below 195 nm. Since this high background absorbance must be subtracted from the spectrum of each protein sample, the resulting difference has a poor signal/noise and greater variability in this wavelength region (λ<195 nm).

### Conformational Stability by Near UV Circular Dichroism Spectroscopy (CD)

Tertiary structure of EFX was assessed by near-UV CD. Signals in the near-UV CD spectra of proteins are associated with aromatic amino acids, and with disulfide bonds located in asymmetric conformational environments, present only when a protein is folded into its distinct 3-dimensional structure.

Near-UV CD spectral measurements (250-350 nm) were obtained on a Chirascan Auto Q100 CD spectrometer (Applied Photophysics Ltd.). Spectra were collected at 20° C. with a protein concentration of 2.0 mg/mL and a pathlength of 5.0 mm. The spectral bandwidth was set to 1.0 nm, the sampling time per point was 1.0 sec, and the step size was 1.0 nm. Ten consecutive scans were averaged for each measurement of a protein sample. A reference spectrum of formulation buffer was recorded prior to measuring the protein sample, and subtracted from the sample's spectrum. After subtraction, CD values were converted to mean residue ellipticity ([Θ]mr) values.

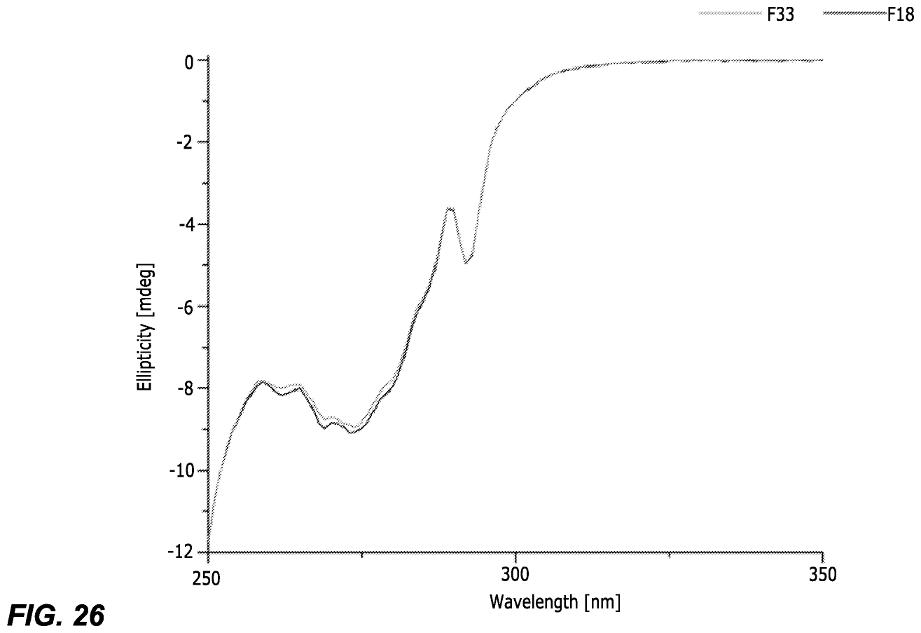

The near UV CD spectra for EFX in formulations F18 and F33 are shown in FIG. 26. The spectra contain significant signals at 289 to 292 nm attributable to tryptophan residues, at 270 to 285 nm corresponding to tyrosine residues, and at 250 to 270 nm attributable to phenylalanine and tyrosine residues superimposed on a broad disulfide signal at 250 to 270 nm. The intensity of these features reflects the unique structural arrangement of disulfide bonds and aromatic amino acids within the folded structure of EFX. For example, if samples of EFX were completely unfolded, the spectrum would be a straight line around zero, while partially unfolded protein would show decreased intensities of the various spectral signals, particularly in the 250-270 nm region associated with spatial arrangement of disulfide bonds. The shape and intensity of the near UV CD spectrum indicate that EFX is folded in a well-defined tertiary structure.

### Thermal Stability by Differential Scanning Microcalorimetry (μDSC)

The thermal stability of EFX was assessed by Differential Scanning Microcalorimetry (μDSC) using a MicroCal Auto VP-Capillary DSC system (Malvern). Thermograms were collected using a protein concentration of 2.0 mg/mL. To characterize the endotherms associated with unfolding of EFX, samples of formulations containing EFX, or excipients and buffer without EFX were heated from 10 to 110° C. at a rate of 60° C./h. To prevent boiling of samples during heating to high temperature, the μDSC cell was pressurized. A baseline run was performed by loading both the reference and sample cells with formulation buffer. The baseline thermogram was subtracted from each measurement of a formulation containing EFX. The excess heat capacity value for a sample was then normalized for protein concentration. Thermal transition midpoint temperature (Tm) values were determined at the center of peaks or shoulders by using derivative analysis of the heating scan. To determine values for thermal stability parameters, data analysis and peak deconvolutions were performed with Origin 7.0 DSC software.

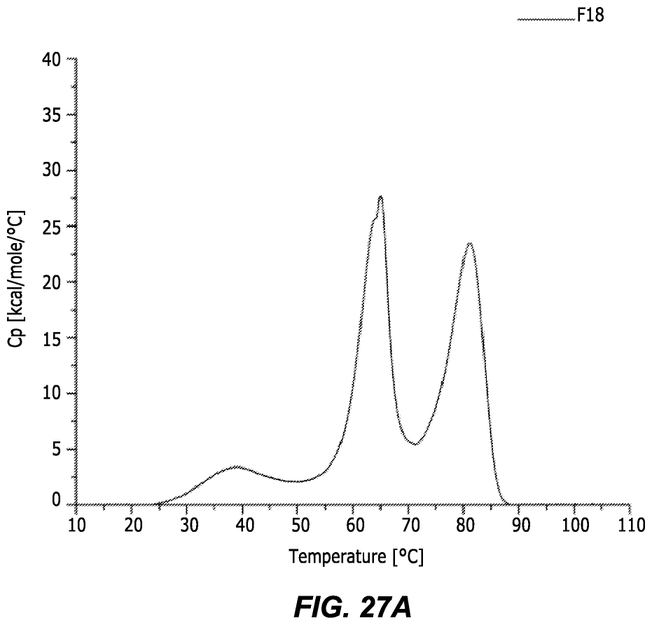

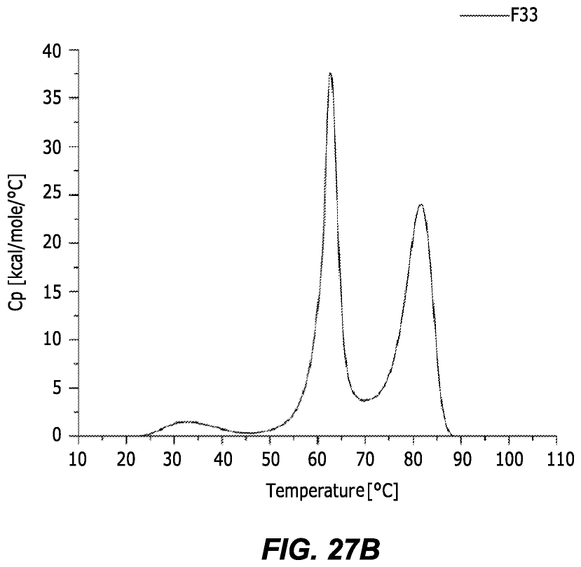

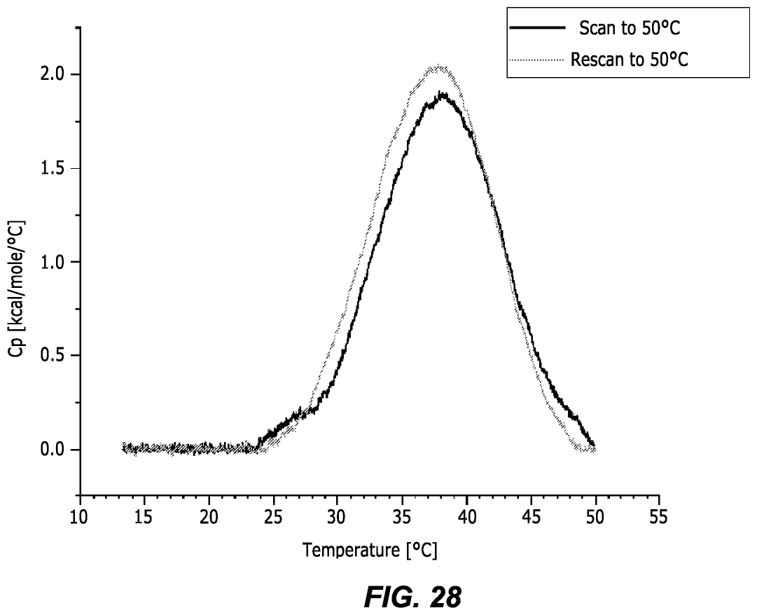

Representative thermograms for EFX in F18 and F33 are shown in FIG. 27. They reveal three discrete endothermic peaks with respective Tm values of approximately 33.8-38.8, 62.7-65.1, and 81.1-81.7° C. (Table 17). The peak at 65° C. includes a prominent left shoulder, indicating the presence of two thermal unfolding events, however, the two peaks could not be resolved by deconvolution approaches. The first endotherm, at 38.8° C., which corresponds to unfolding of FGF21 domains, is fully reversible. FIG. 28 shows an initial thermogram of EFX when heated to 50° C., overlayed with a second thermogram obtained after cooling to 10° C., then reheating to 50° C. Superimposable thermograms from these two consecutive thermal melts confirm the first unfolding (Tm1=38.8° C.) is fully reversible.

TABLE 17
Results of μDSC Measurements of EFX F18 and F33.

μDSC results

| | Tₘ₁ | Tₘ₂ | Tₘ₃ | ΔH |
|---|---|---|---|---|
| Sample | [° C.] | [° C.] | [° C.] | [kcal/mol] |
| F18 | 38.8 | 65.1 | 81.1 | 412 |
| F33 | 33.8 | 62.7 | 81.7 | 408 |

Tm = thermal transition midpoint temperature.
ΔH = enthalpy change.

μDSC method demonstrated unfolding onset at approximately 28° C. which suggests EFX is conformationally unstable. As a protein unfolds, amino acid residues may become more exposed on an external surface, potentially resulting in deamidation of Asp and Gln and oxidation of Met, while exposure of more hydrophobic amino acids may trigger protein aggregation. Formulations containing Arg/Arg-HCl, Glu, and Lys appear to improve EFX conformational stability thereby reducing physical degradation, aggregation, and charge variants formation. Formulations containing sucrose appear also to have better viscoelastic properties, conformational and thermal stability than those containing trehalose.

### Example 5: Lyophilization Process

This Example describes the development of a formulation that is stable at room temperature and enables patients to self-administer EFX. Eleven formulations of EFX, including F2, F3, F7, F9, F12, F14, F15, F16, F17, F18, and F33 were lyophilized in vials and dual chamber devices. Various lyophilization process designs/cycles (parameters) were evaluated to improve/optimize process robustness, consistency of critical quality attributes (CQA) and long-term stability at room temperature. To ensure ease of self-administration by patients, time to reconstitute the lyophile was measured for various formulations and lyophilization process set parameters.

### Lyophilization Process Description

The freeze-drying lyophilization process/cycle was varied to evaluate the effect of process steps and process performance parameters on CQA, including reconstitution time, appearance of lyophilized cake, subvisible particles counts post reconstitution, and stability during long-term storage.

### Initial Lyophilization Process/Cycle

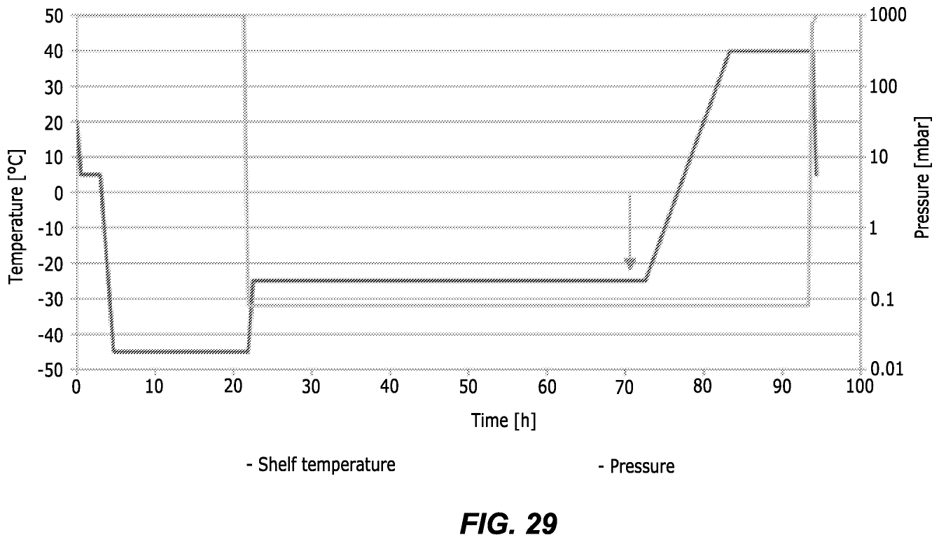

An example of a lyophilization process for vials without an annealing step is shown in FIG. 29. Shelves of the freeze drier are pre-cooled at 5° C. with the initial freezing cycle following at −45° C. Subsequently, primary drying is conducted at −25° C. shelf temperature and 0.08 mBar chamber pressure. During primary drying, heat is applied to the product to convert phase-separated ice directly to water vapor by sublimation. The end of primary drying (denoted with an arrow on FIG. 29) is defined as the point when the capacitance gauge and the Pirani vacuum sensor are aligned with product temperature stabilized above shelf temperature. While primary drying removes ice crystals by sublimation, secondary drying is required to remove bound to EFX water by diffusion. While secondary drying occurs to some degree during sublimation, the desorption rate of water at low shelf temperatures (typical for primary drying) is low. By raising shelf temperature to 40° C. during a subsequent secondary drying cycle, adsorbed water is completely removed after 10 hours.

### Development of Lyophilization Process

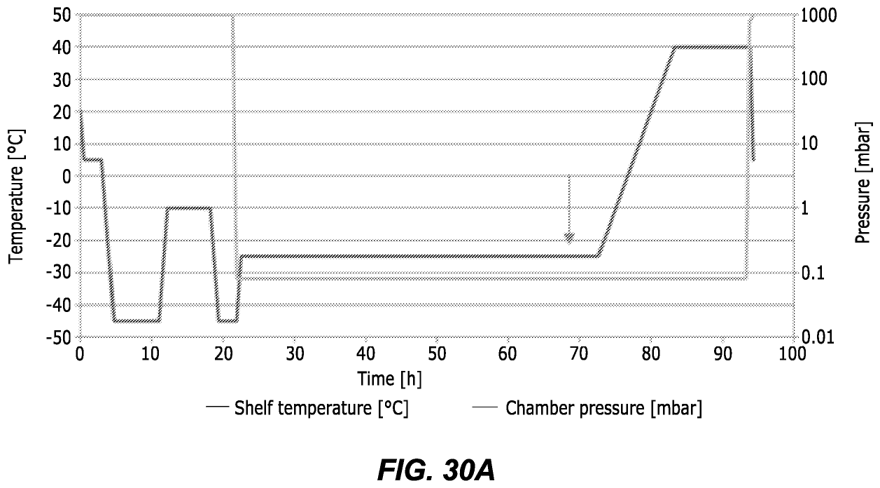

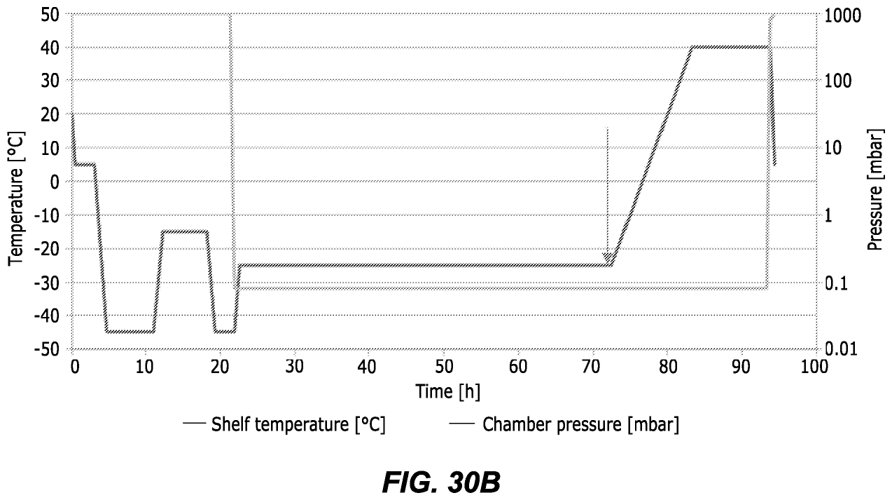

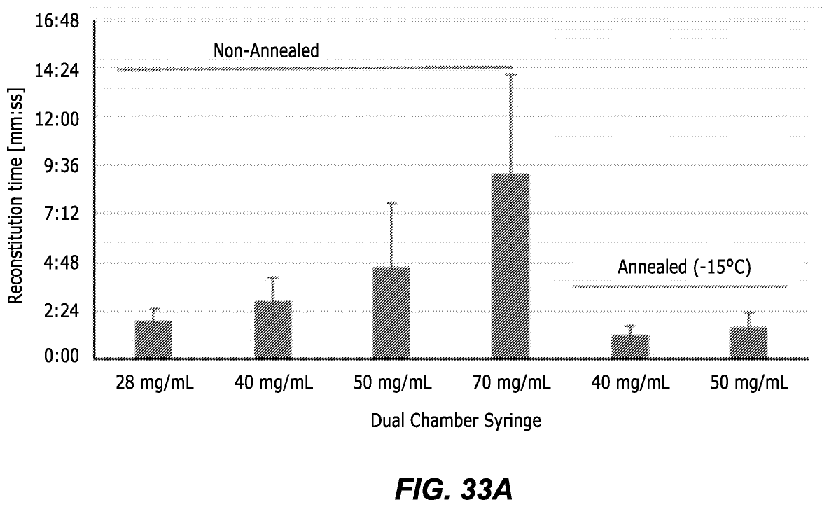

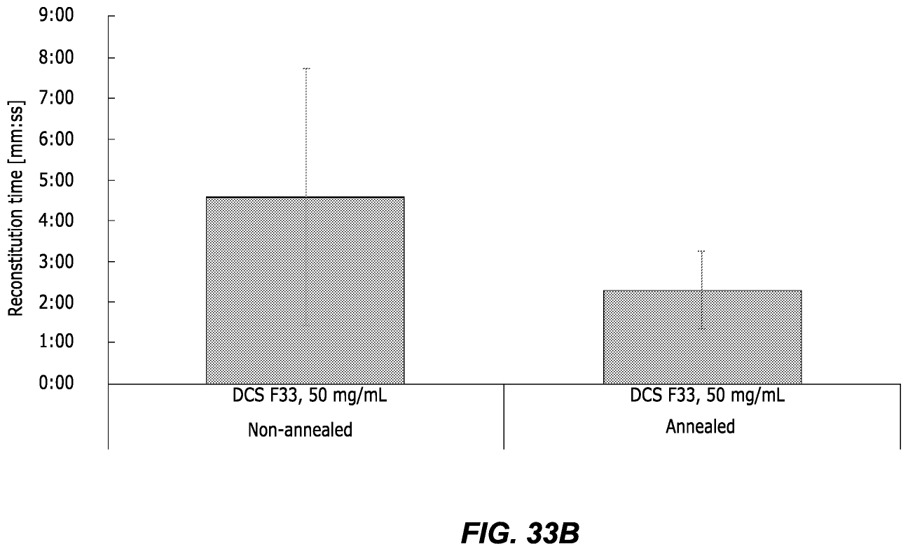

Variations of the lyophilization process were explored. Notably, the initial freezing cycle was adapted to include an annealing step in which the shelf temperature cycles up and down during the freezing cycle. Annealing serves two different purposes, depending on the design of the formulation: a) it allows growth of crystals for phase-separated, crystallizing excipients, and b) it increases the size of ice crystals through Ostwald ripening, which results in larger pores sizes, thereby increasing the rate of sublimation during primary drying. The annealing steps in this study explored annealing at −10° C. for 5 h (FIG. 33), annealing at −7° C. or −5° C. for 10 h (not shown). On completion of the annealing step, the shelves were cooled to −45° C. and the lyophilization process continued with the primary drying step at −25° C. (FIG. 30).

### Impact of Incorporating an Annealing Cycle in the Freeze-Drying Process on Reconstitution Time of Lyophilized EFX

The lyophilized dry powder cakes of formulations containing EFX were reconstituted with water for injection and compounded diluent based on formulation F33. For ease of use by patients, reconstitution time should ideally be 5 minutes or less (although this is not required in the context of the disclosure).

To take account of the different dry solid content of the formulations, a gravimetric determination of the water loss during lyophilization was performed. To do so, 10 vials of each formulation were weighed before and after lyophilization, and the water loss was calculated. Subsequently, the determined volume of water for injection was used to reconstitute the dry powder cakes. Water was added into the center of the container closures (vial or Dual Chamber Device, DCD) using a pipette. The container closure was carefully swirled (shaking was avoided). The time taken for the lyophilized cake to be fully reconstituted was recorded (hours: minutes: seconds or minutes: seconds).

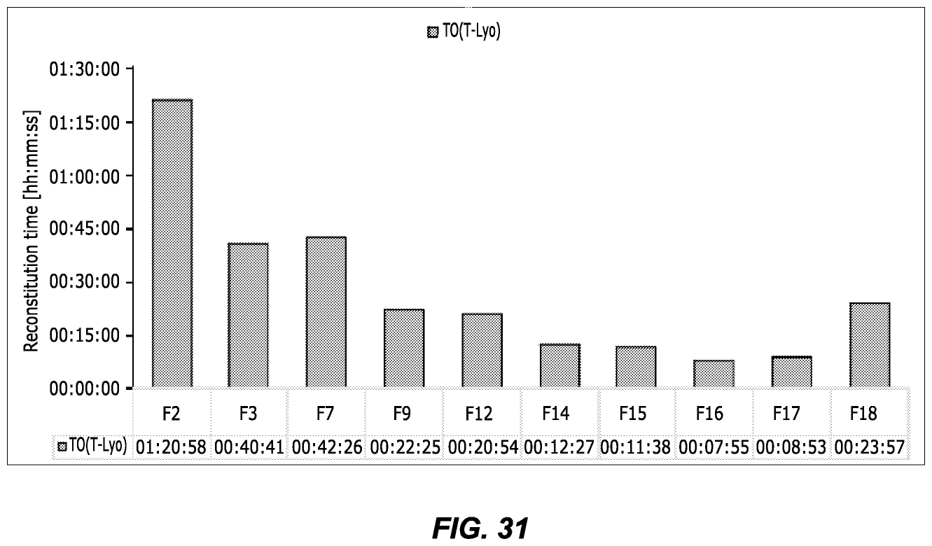

On completion of the freeze-drying process without an annealing step (FIG. 29), reconstitution times were recorded for ten lyophilized formulations corresponding to 100 mg/mL EFX, including F2, F3, F7, F9, F12, F14, F15, F16, F17, and F18, see FIG. 31.

The measured reconstitution times of the selected ten formulations varied widely ranging from 1 hour: 20 min: 58 seconds for F2, to 7 min: 55 seconds for F16, indicating dependence on the composition and pH of formulations. Notably, reconstitution times were significantly longer in formulations like F2, in which EFX is susceptible to forming structured gel lattices over time. Such formulations are characterized by high viscosities (FIG. 4) and non-Newtonian behavior (FIG. 5). Longer reconstitution times were also evident with formulations compounded at a pH below the isoelectric point of EFX.

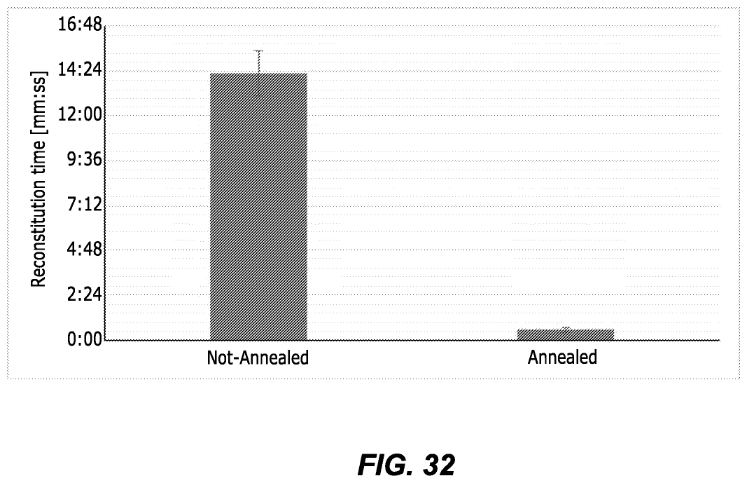

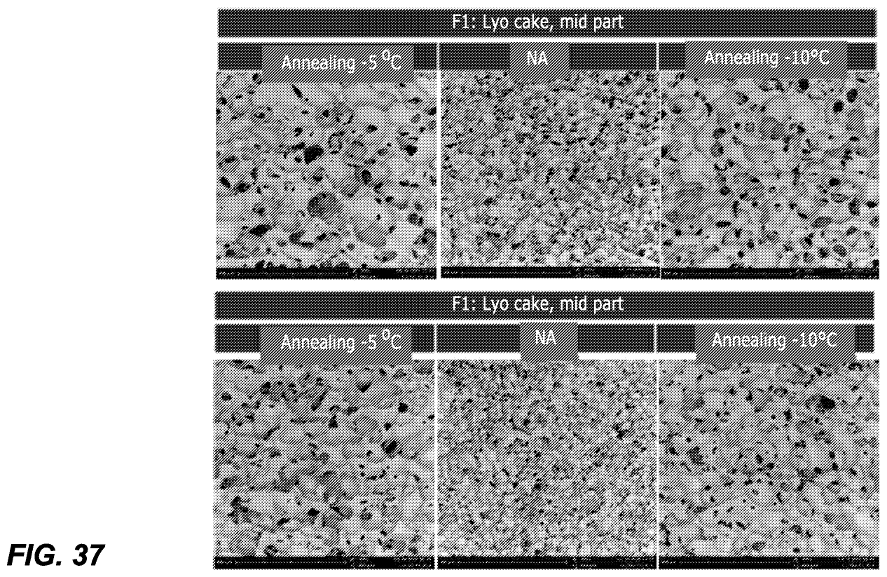

Incorporation of an annealing step in the lyophilization process (FIG. 30) significantly improved the structure of lyophilized cake by decreasing specific surface area (less dense cakes) and maximizing pore size area (FIG. 37), and accelerated reconstitution by approximately 50% for a given formulation, e.g., F33, in vials (FIG. 32) and dual chamber devices (FIG. 33).

### Specific Surface Area (BET)

The effect of an annealing step on the structure of lyophilized cake was evaluated by Specific Surface Area BET analysis. The specific surface area of selected freeze-dried formulations was analyzed by the BET (Brunauer, Emmett and Teller theory) method using an Autosorb-1 (Quantachrome Instruments). The BET method is the most widely used procedure for the determination of surface area of solid materials and uses the following equation:

$$\frac{1}{W ( ( \frac{p 0}{p} ) - 1 )} = \frac{1}{Wm * C} + \frac{C - 1}{Wm * C} ( \frac{p}{p 0} )$$

W: weight of gas adsorbed at a relative pressure (p/p0) [g];
p: partial vapor pressure [Pa] of adsorbate gas in equilibrium with the surface at 77.3 Kelvin,
p0: saturated pressure [Pa] of adsorbate gas
Wm: weight of adsorbate constituting a monolayer of surface coverage [g]
C: BET constant, is related to the energy of adsorption in the first adsorbed layer

The BET equation requires a linear plot of 1/[W(p0/p)-1] vs. p/p0 which for most solids, is restricted to a limited region of the adsorption isotherm, usually in the p/p0 range of 0.05 to 0.35. The standard multipoint BET procedure requires a minimum of three points in the appropriate relative pressure range.

The weight (Wm) of monolayer can be obtained from the slope (s) and intercept (i) of the BET plot:

$$Wm = \frac{1}{s + i}$$

The second step in the application of the BET method is the calculation of the surface area. This requires knowledge of the molecular cross-sectional area (Acs) of adsorbate molecule. The total surface area (St) of sample can be expressed as:

$$St = \frac{Wm * N * Acs}{M}$$

N: Avogadro's number (6.0221415×10²³ molecules/mol)
M: Molecular mass of the adsorbate [g/mol]

The specific surface area (S) [m²/g] of solid can be calculated from total surface area (St [m²]) and sample weight (w [g]):

$$S = St / w$$

### Sample Preparation and Analysis

Approximately 100 mg freeze-dried product were carefully crushed into small pieces with a spatula, and transferred to the measuring vessel. Before measuring the area by adsorption of krypton, all gas adsorbed from the environment must be removed from the surface of the sample. The measuring vessel was attached to a degasser station, and the vacuum switched on. Vacuum was discontinued after 16 hours degassing at room temperature.

Subsequently the measuring vessels (cells) were filled for about 5 seconds with helium (0.7-1.0 bar). Adsorption of krypton was measured at −195.8° C. (77.3 K) bath temperature. Seven data points covering a p/p0 region of 0.05 to 0.35 were collected. The 1/[W*(p0/p)-1] was plotted against p/p0.

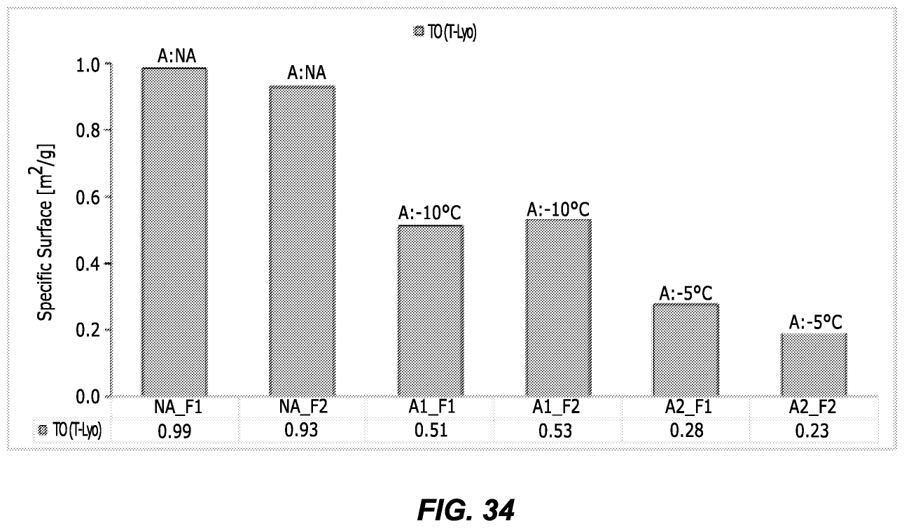

The Specific Surface Area (BET) of lyophilized cake produced from formulation F33, with and without annealing steps incorporated into the freeze-drying process, is presented in FIG. 34. The numerical values for Specific Surface Area were 50% and 75% lower respectively for cakes produced using either an annealing step of −5° C. for 10 hours (A1 process design) or −10° C. for 5 hours (A2 process design) compared to cakes produced without an annealing step during freeze drying (NA process design), see FIG. 34. The data demonstrates that incorporation of an annealing step in the freeze-drying process significantly decreases specific surface area of the resulting cake (less dense cakes), which is associated with significantly shorter reconstitution times such as for F33 (FIGS. 33A & 33B).

Evaluation of Morphology and Structure of Lyophilized Cakes by Scanning Electron Microscopy with Energy Dispersive X-Ray Spectroscopy (SEM-EDX)

Morphology of freeze-dried formulation containing EFX was analyzed by SEM-EDX method, using a JSM-IT 200 (Jeol) system. Sample preparation was performed in a glove box under controlled humidity (≤10% r.h.). The number of pores in lyophilized cake were counted, as well as the area of pores using the signal from the secondary electron detector (SED). The landing voltage and probe current were set at 5 kV and 20%, respectively. All measurements were conducted under uncontrolled high vacuum after 20 minutes of equilibration. Brightness and contrast were adjusted to achieve a high contrast in the resulting images. Enumeration of pores and quantification of pore area was conducted using Jeol particle analysis software V3. A two level binarization (conversion of multi-tone image into black and white), based on the brightness value of the acquired images (which was fine-tuned for each sample), was applied to identify the pores. Pores with an area smaller than 20 μm² were excluded from the analysis.

SEM analyses were also completed using the bench top scanning electron microscope (SEM) Phenom (Phenom-World B.V.). The instrument was equipped with a CCD camera and a diaphragm vacuum pump. Illumination of the sample, as well as resolution of the spherical particles of the reference sample was confirmed.

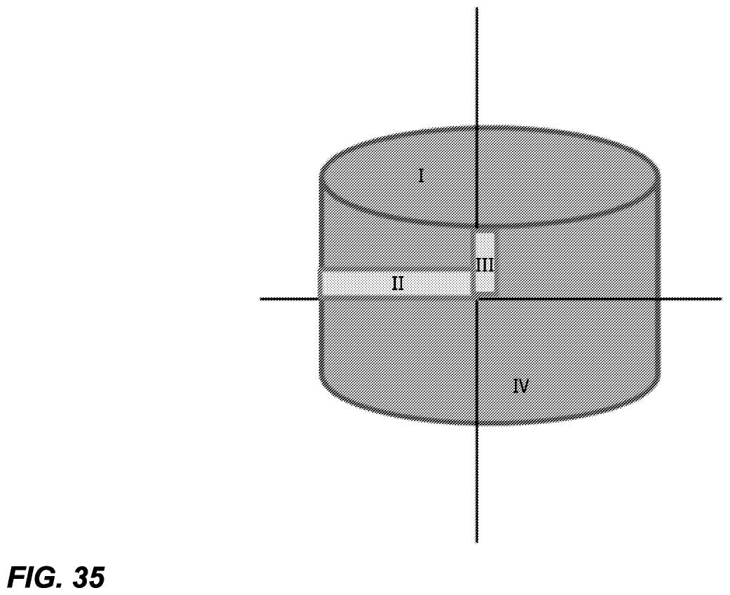

To recover freeze-dried cake, glass vials containing cake were cut horizontally in the middle using a Micromot 50/E equipped with a diamond grinding wheel, Proxxon. Horizontal and vertical slices of freeze-dried cake were prepared using a razor blade, as shown in FIG. 35.

Slices were placed on carbon conductive cement on a sample holder, presenting the cut of the cross section as the top surface as listed in Table 18.

TABLE 18
Overview of the sections of freeze-
dried cake analyzed by SEM-EDX.

| Section number | Description |
|---|---|
| I | Top of the freeze-dried cake |
| II | Horizontal cut |
| III | Vertical cut |
| IV | Bottom of the freeze-dried cake |

The EFX dry powder cakes were analyzed under vacuum with a light optical magnification of 20× and 5 kV acceleration voltage. The electron optical magnification was adjusted to between 340× and 10,000×, with images collected from representative sections of each sample. The lowest magnification was dependent on the height of the sample and positioning inside the SEM.

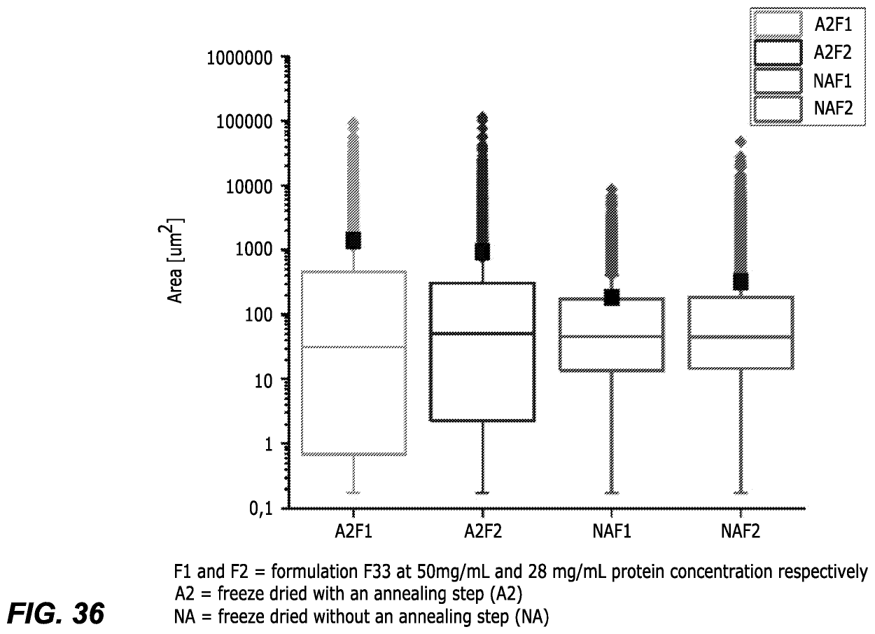

Box-and-whisker plots of the distribution of pore area of the cross-sections of the lyophilized cakes produced from formulation F33, with and without an annealing step in the freeze-drying process and analyzed by SEM-EDX are shown in FIG. 36. Broader distributions of pore area are evident in cakes produced by freeze drying with an annealing step compared to a process without an annealing step.

Consistent with this, the SEM-images in FIG. 37 demonstrate that incorporation of an annealing step in the lyophilization process significantly improves dry powder cake structure and morphology by decreased specific surface area (less dense cakes) and maximized pore size area facilitating primary and secondary drying process steps and improving reconstitution times.

### Example 6: Long-Term Stability of Lyophilized Formulations of EFX Stored Under Stress Conditions

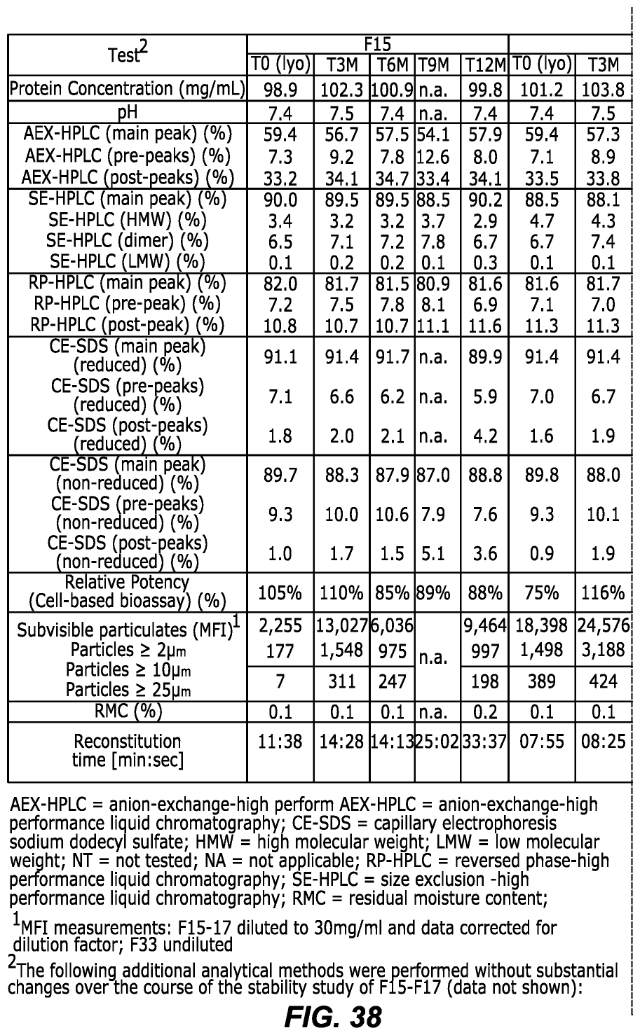

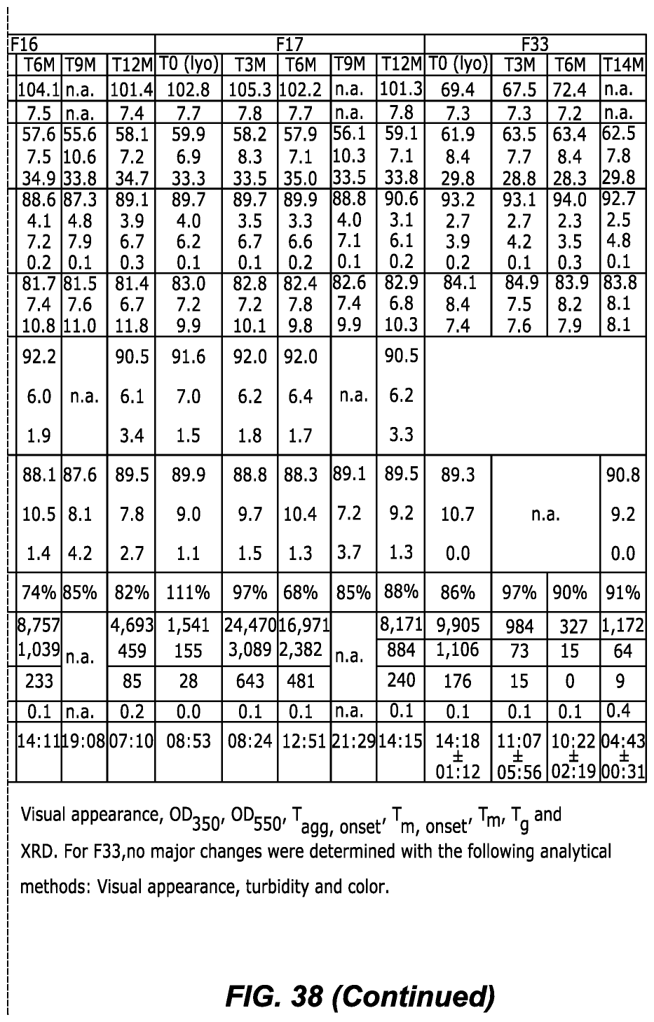

Lyophilized formulations in the pH range 7.3-7.8 (F15, F16, F17, and F33) were selected and stored under room temperature conditions (25° C./60% Relative Humidity). To assess stability after storage for up to 12 months, the formulations were evaluated against a panel of tests employed for QC release of EFX drug product. FIG. 38, summarizes the data for tests indicative of long term stability of lyophilized EFX at 25° C. in Formulations F15, F16, F17, and F33.

Comparing charge variants, size variants (aggregation, clipping/fragmentation), subvisible particle formation by MFI, and cell-based potency of EFX in all 4 formulations at time zero, three months, six months (not shown), nine months, and 14 months, the data demonstrated essentially no change or minimal change, allowing for method variability, in the numerical values of critical product attributes over time for lyophilized formulations, including F33, within this pH range.

The lyophilization process incorporating an annealing step in the freezing cycle not only improved process robustness, but also resulted in consistent product attributes, long-term stability under various conditions including room temperature, as well as rapid reconstitution enabling ease of self-administration by patients.

These observations were applicable not only to EFX lyophilized in the most stable formulation F33, but also to other formulations that had previously demonstrated significant rates of formation of charge variants, size variants (HMWS and LMWS), and subvisible particles when stored as liquid under refrigerated and room temperature conditions.

### Example 7: Serum Concentrations of EFX

Pharmacokinetic parameters of various formulations in vivo were examined. Each animal in Groups 1-7 received a single subcutaneous (SC) dose (volume of 5 mL/kg) of the appropriate test material formulation comprising EFX. The details of the EFX formulation administered to each group are provided in Table 19. Each group comprised nine females, and the dose administered to each subject was 100 mg/kg.

TABLE 19
Formulations utilized in study

| | | Viscosity |
|---|---|---|
| Group | Formulation | (cP at 20° C.) |
| 1 | 20 mM Tris-HCl, 120 mM Sucrose, 120 mM Arginine/Arginine-HCl, | 1.9 |
| | 0.06% w/v polysorbate 20, pH 7.3 ± 0.3 in Sterile Water for Injection. | |
| 2 | 20 mM Na glutamate/glutamic acid, 200 mM trehalose, 40 mM Lys- | 9920 |
| | HCl, 0.04% polysorbate 20 (started at 0.008% w/v), pH 5.5 (F4) | |
| 3 | 20 mM Na succinate/succinic acid, 220 mM trehalose, 0.04% | 9279 |
| | polysorbate 20 (started 0.008% w/v), pH 5.5 (F7) | |
| 4 | 20 mM Na succinate/succinic acid, 220 mM sucrose, 0.04% | 3878 |
| | polysorbate 20 (started 0.008% w/v), pH 5.5 (F9) | |
| 5 | 20 mM Histidine, 220 mM Trehalose, 0.04% polysorbate 20 (started | >instrument |
| | 0.008% w/v), pH 6.0 (F11) | upper limit |
| 6 | 20 mM Tris-HCl, 80 mM Arg/Arg-HCl, 80 mM sucrose, 0.5% Na- | 5.06 |
| | CMC, 0.06% w/v polysorbate 20, pH 7.3 | |
| 7 | 20 mM Tris-HCl, 80 mM Arg/Arg-HCl, 80 mM sucrose, 0.5% PEG- | 1.94 |
| | 4000, 0.06% w/v polysorbate 20, pH 7.3 | |

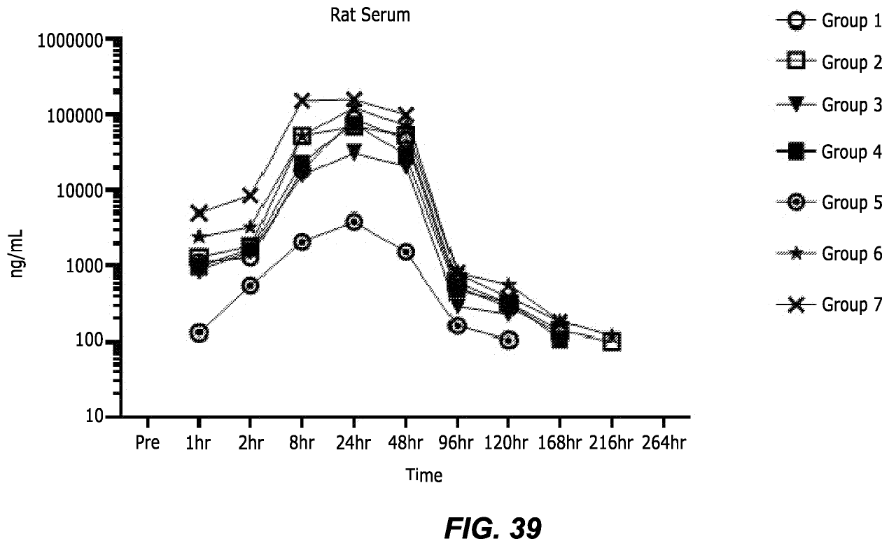

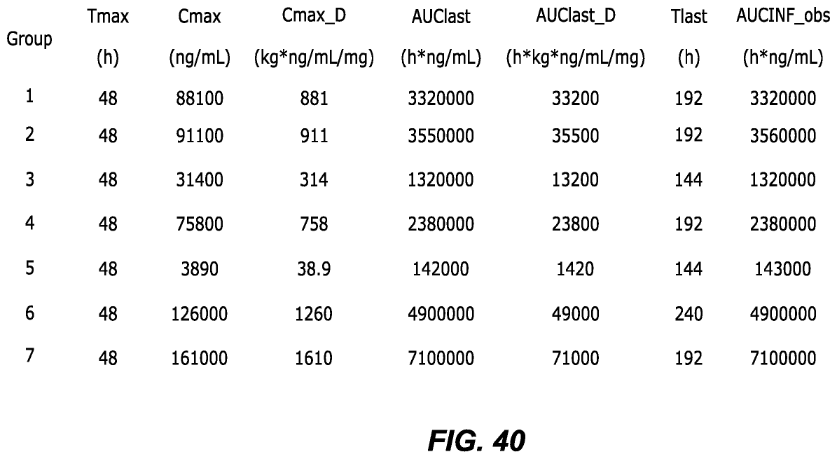

The concentration of EFX at various timepoints post-administration is illustrated in FIG. 39. FIG. 40 provides a summary of pharmacokinetic parameters indicative of overall systemic exposure (AUC), providing an indication of bioavailability, as well as highest concentration in systemic circulation (Cmax). Surprisingly, PEG (namely PEG 4000) increased systemic exposure/bioavailability following subcutaneous injection, despite not being covalently conjugated to EFX.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

1. A pharmaceutical composition comprising:
(1) Efruxifermin (EFX);
(2) a sugar;
(3) about 20 to about 200 mM arginine/arginine-HCl or arginine/glutamic acid; and
(4) a surfactant;
wherein the composition has a pH from about 6.9 to about 8.1.

2. The composition of claim 1, wherein the EFX concentration is about 25 to about 150 mg/ml.

3. The composition of claim 1, wherein the EFX concentration is about 28 mg/ml, about 50 mg/ml, about 70 mg/ml, or about 100 mg/ml.

4.-7. (canceled)

8. The composition of claim 1, comprising about 20 mM to about 200 mM arginine/arginine-HCl.

9. The composition of claim 8, comprising about 120 mM arginine/arginine-HCl.

10. The composition of claim 1, comprising arginine/arginine-HCl at a ratio of about 1:30 arginine/arginine-HCl to about 1:50 arginine/arginine-HCl.

11. (canceled)

12. The composition of claim 1, comprising about 20 mM to about 200 mM arginine/glutamic acid.

13. The composition of claim 1, further comprising Tris-HCl, sodium phosphate, sodium succinate/succinic acid, sodium glutamate/glutamic acid, sodium acetate/acetic acid, glycylglycine/glycylglycine-HCl, histidine, or citrate buffer.

14. The composition of claim 13, comprising Tris-HCl at a concentration of about 10 mM to about 50 mM.

15. The composition of claim 1, wherein the sugar is sucrose, glucose, fructose, or maltose.

16. The composition of claim 15, wherein the sucrose concentration is about 50 to about 220 mM.

17. The composition of claim 15, wherein the sucrose concentration is about 120 mM.

18. (canceled)

19. The composition of claim 1, wherein the surfactant is polysorbate-20 or polysorbate-80.

20. The composition of claim 19, wherein the surfactant concentration is about 0.004% to about 0.1% w/v.

21. The composition of claim 1, wherein the composition has a pH of about 7.3.

22. The composition of claim 1, wherein the composition has a viscosity of ≤5 cP at room temperature.

23. (canceled)

24. The composition of claim 1 comprising:
(1) about 25-150 mg/mL Efruxifermin (EFX);
(2) about 120 mM sucrose;
(3) about 120 mM Arginine/Arginine-HCl;
(4) about 0.06% weight/volume (w/v) polysorbate-20; and
(5) about 20 mM Tris-HCl;
wherein the composition has a pH of about 7.3.

25.-28. (canceled)

29. The composition of claim 1, wherein (a) the composition is stable at a temperature of about 2-8° C. for at least 21 months as a liquid, (b) the composition comprises no more than about 40% EFX acidic charged variant species when stored between −30° C. to −20° C. for up to 24 months, (c) the composition comprises no more than about 40% EFX acidic charged variant species when stored at about 2-8° C. for up to 9 months, (d) the composition comprises no more than about 40% EFX acidic charged variant species when stored at about 25° C. for up to 4 weeks, (e) the composition comprises no more than about 20% EFX size variant species at about 25° C. for up to 4 weeks, or (f) the composition comprises no more than about 10% EFX size variant species when stored at about 2-8° C. for up to 14 months.

30.-34. (canceled)

35. The composition of claim 1, which is a lyophilized composition.

36. The composition of claim 35, comprising a residual moisture content of about 1% or less.

37.-40. (canceled)

41. The composition of claim 1, wherein the composition further comprises carboxymethyl cellulose or hydroxypropyl methylcellulose.

42. The composition of claim 41, wherein (i) the carboxymethyl cellulose is sodium carboxymethyl cellulose or (ii) the hydroxypropyl methylcellulose is sodium hydroxypropyl methylcellulose.

43. The composition of claim 42, wherein the sodium carboxymethyl cellulose is present in a concentration of about 0.05% to about 5%.

44. The composition of claim 43, wherein the sodium carboxymethyl cellulose is present in a concentration of about 0.5%.

45. The composition of claim 29, comprising about 80 mM arginine/glutamic acid and about 80 mM sucrose.

46. A method comprising (a) reconstituting the composition of claim 35 within about five minutes to obtain a reconstituted composition and (b) administering the reconstituted composition to a subject.

47.-50. (canceled)

51. A process for preparing a lyophilized composition, the process comprising:
(a) freezing the composition of claim 1;
(b) annealing the composition of step (a) at a temperature of about −5° C. to about −20° C.;
(c) primary drying the product of step (b); and
(d) secondary drying the product of step (c).

52.-56. (canceled)

57. A method of treating nonalcoholic steatohepatitis (NASH) or nonalcoholic fatty liver disease (NAFL), comprising administering the pharmaceutical composition of claim 1 to a subject in need thereof.

58. A method of reversing NASH with cirrhosis, comprising administering the pharmaceutical composition of claim 1 to a subject in need thereof.

59. A method of treating alcoholic steatohepatitis (ASH), alcoholic liver disease (ALD), alcoholic fatty liver disease (AFL), type 2 diabetes, obesity, dyslipidemia, craving, gout, addiction, or a protein misfolding disease comprising administering the pharmaceutical composition of claim 1 to a subject in need thereof.

60. A method of normalizing liver fat content, lowering blood glucose, increasing insulin sensitivity, or reducing uric acid in a subject in need thereof, comprising administering the pharmaceutical composition of claim 1 to a subject in need thereof.

61. (canceled)

62. A method of reversing liver cirrhosis or reducing fibrosis associated with NASH, ASH, ALD, AFL, or protein misfolding disease, comprising administering the pharmaceutical composition of claim 1 to a subject in need thereof.

63.-85. (canceled)
